SEQUENCE LISTING (9 sequences)

SEQ ID NO: 1 - DNA, 2648 bp, Homo sapiens
gactcctaggggcttgcagactagtgggagagaaagaacatcgcagcagccaggcagaaccaggacaggtgaggtgcaggctggctttcctctcgcagcgcggtgtggagtcctgtcctgcctcagggcttttcggagcctggatcctcaaggaacaagtagacctggccgcggggagtggggagggaaggggtgtctattgggcaacagggcggggcaaagccctgaataaaggggcgcagggcaggcgcaagtggcagagccttcgtttgccaagtcgcctccagaccgcagacatgaaacttgtcttcctcgtcctgctgttcctcggggccctcggactgtgtctggctggccgtaggaggagtgttcagtggtgcgccgtatcccaacccgaggccacaaaatgcttccaatggcaaaggaatatgagaaaagtgcgtggccctcctgtcagctgcataaagagagactcccccatccagtgtatccaggccattgcggaaaacagggccgatgctgtgacccttgatggtggtttcatatacgaggcaggcctggccccctacaaactgcgacctgtagcggcggaagtctacgggaccgaaagacagccacgaactcactattatgccgtggctgtggtgaagaagggcggcagctttcagctgaacgaactgcaaggtctgaagtcctgccacacaggccttcgcaggaccgctggatggaatgtccctatagggacacttcgtccattcttgaattggacgggtccacctgagcccattgaggcagctgtggccaggttcttctcagccagctgtgttcccggtgcagataaaggacagttccccaacctgtgtcgcctgtgtgcggggacaggggaaaacaaatgtgccttctcctcccaggaaccgtacttcagctactctggtgccttcaagtgtctgagagacggggctggagacgtggcttttatcagagagagcacagtgtttgaggacctgtcagacgaggctgaaagggacgagtatgagttactctgcccagacaacactcggaagccagtggacaagttcaaagactgccatctggcccgggtcccttctcatgccgttgtggcacgaagtgtgaatggcaaggaggatgccatctggaatcttctccgccaggcacaggaaaagtttggaaaggacaagtcaccgaaattccagctctttggctcccctagtgggcagaaagatctgctgttcaaggactctgccattgggttttcgagggtgcccccgaggatagattctgggctgtaccttggctccggctacttcactgccatccagaacttgaggaaaagtgaggaggaagtggctgcccggcgtgcgcgggtcgtgtggtgtgcggtgggcgagcaggagctgcgcaagtgtaaccagtggagtggcttgagcgaaggcagcgtgacctgctcctcggcctccaccacagaggactgcatcgccctggtgctgaaaggagaagctgatgccatgagtttggatggaggatatgtgtacactgcaggcaaatgtggtttggtgcctgtcctggcagagaactacaaatcccaacaaagcagtgaccctgatcctaactgtgtggatagacctgtggaaggatatcttgctgtggcggtggttaggagatcagacactagccttacctggaactctgtgaaaggcaagaagtcctgccacaccgccgtggacaggactgcaggctggaatatccccatgggcctgctcttcaaccagacgggctcctgcaaatttgatgaatatttcagtcaaagctgtgcccctgggtctgacccgagatctaatctctgtgctctgtgtattggcgacgagcagggtgagaataagtgcgtgcccaacagcaacgagagatactacggctacactggggctttccggtgcctggctgagaatgctggagacgttgcatttgtgaaagatgtcactgtcttgcagaacactgatggaaataacaatgaggcatgggctaaggatttgaagctggcagactttgcgctgctgtgcctcgatggcaaacggaagcctgtgactgaggctagaagctgccatcttgccatggccccgaatcatgccgtggtgtctcggatggataaggtggaacgcctgaaacaggtgttgctccaccaacaggctaaatttgggagaaatggatctgactgcccggacaagttttgcttattccagtctgaaaccaaaaaccttctgttcaatgacaacactgagtgtctggccagactccatggcaaaacaacatatgaaaaatatttgggaccacagtatgtcgcaggcattactaatctgaaaaagtgctcaacctcccccctcctggaagcctgtgaattcctcaggaagtaaaaccgaagaagatggcccagctccccaagaaagcctcagccattcactgcccccagctcttctccccaggtgtgttggggccttggcctcccctgctgaaggtggggattgcccatccatctgcttacaattccctgctgtcgtcttagcaagaagtaaaatgagaaattttgttgatattctctccttaaaaaaaaaaaaaaaaaaaaaaaaaaaaaa

SEQ ID NO: 2 - protein, 692 aa, Homo sapiens
GRRRRSVQWCAVSQPEATKCFQWQRNMRKVRGPPVSCIKRDSPIQCIQAIAENRADAVTLDGGFIYEAGLAPYKLRPVAAEVYGTERQPRTHYYAVAVVKKGGSFQLNELQGLKSCHTGLRRTAGWNVPIGTLRPFLNWTGPPEPIEAAVARFFSASCVPGADKGQFPNLCRLCAGTGENKCAFSSQEPYFSYSGAFKCLRDGAGDVAFIRESTVFEDLSDEAERDEYELLCPDNTRKPVDKFKDCHLARVPSHAVVARSVNGKEDAIWNLLRQAQEKFGKDKSPKFQLFGSPSGQKDLLFKDSAIGFSRVPPRIDSGLYLGSGYFTAIQNLRKSEEEVAARRARVVWCAVGEQELRKCNQWSGLSEGSVTCSSASTTEDCIALVLKGEADAMSLDGGYVYTAGKCGLVPVLAENYKSQQSSDPDPNCVDRPVEGYLAVAVVRRSDTSLTWNSVKGKKSCHTAVDRTAGWNIPMGLLFNQTGSCKFDEYFSQSCAPGSDPRSNLCALCIGDEQGENKCVPNSNERYYGYTGAFRCLAENAGDVAFVKDVTVLQNTDGNNNEAWAKDLKLADFALLCLDGKRKPVTEARSCHLAMAPNHAVVSRMDKVERLKQVLLHQQAKFGRNGSDCPDKFCLFQSETKNLLFNDNTECLARLHGKTTYEKYLGPQYVAGITNLKKCSTSPLLEACEFLRK

SEQ ID NO: 3 - DNA, 2076 bp, Artificial Sequence
  Recombinant sequence generated in laboratory
gggcggcggcggcgctcggtgcagtggtgcgccgtgtcccagcccgaggcgaccaagtgcttccagtggcagcgcaacatgcggaaggtgcgcggcccgccggtcagctgcatcaagcgggactcccccatccaatgcatccaggccatcgcggagaaccgcgccgacgcggtcaccctggacggcgggttcatctacgaggcggggctcgccccgtacaagctccgcccggtggcggcggaggtgtacggcaccgagcgccagccgcgcacgcactactacgcggtggccgtcgtcaagaagggcgggtccttccagctcaacgagctgcagggcctgaagtcgtgccacacgggcctccggcggacggcgggctggaacgtgcccatcggcaccctgcgccccttcctgaactggaccggcccgccggagccgatcgaggccgccgtggcccgcttcttcagcgcctcctgcgtccccggcgccgacaagggccagttcccgaacctctgccggctctgcgccgggacgggcgagaacaagtgcgccttctcctcgcaggagccgtacttctcctactcgggcgcgttcaagtgcctccgcgacggggccggcgacgtggcgttcatccgcgagtccaccgtgttcgaggacctctccgacgaggcggagcgggacgagtacgagctgctgtgccccgacaacacccgcaagccggtggacaagttcaaggactgccacctggcgcgggtgccctcgcacgcggtcgtcgcccgcagcgtcaacggcaaggaggacgcgatctggaacctcctccgccaggcccaggagaagttcggcaaggacaagtcccccaagttccagctcttcgggagccccagcggccagaaggacctcctcttcaaggactccgcgatcggcttctcccgcgtccccccgcgcatcgactccggcctgtacctcggctccgggtacttcaccgcgatccagaacctccggaagagcgaggaggaggtggcggcgcggcgggcccgcgtcgtgtggtgcgccgtgggcgagcaggagctgcggaagtgcaaccagtggagcggcctgagcgaggggtcggtgacctgctcgtccgccagcaccaccgaggactgcatcgcgctcgtcctcaagggggaggccgacgcgatgagcctcgacggggggtacgtctacaccgccggcaagtgcggcctggtcccggtcctggcggagaactacaagtcgcagcagtccagcgaccccgacccgaactgcgtggaccgccccgtcgagggctacctcgccgtggccgtcgtgcgccggtccgacacctccctgacgtggaacagcgtcaagggcaagaagagctgccacaccgccgtggaccgcaccgccggctggaacatcccgatgggcctcctcttcaaccagaccggctcctgcaagttcgacgagtacttctcccagtcctgcgcccccggctcggacccccgctccaacctgtgcgccctctgcatcggggacgagcagggcgagaacaagtgcgtgcccaacagcaacgagcggtactacggctacacgggggccttccgctgcctggcggagaacgccggggacgtcgcgttcgtgaaggacgtgaccgtgctgcaaaacacggacgggaacaacaacgaggcgtgggcgaaggacctcaagctcgccgacttcgccctgctgtgcctcgacggcaagcgcaagcccgtcaccgaggcgcggtcctgccacctggcgatggcccccaaccacgccgtcgtctcccgcatggacaaggtcgagcgcctcaagcaggtgctcctgcaccagcaggccaagttcggccggaacggcagcgactgcccggacaagttctgcctgttccagtcggagaccaagaacctcctcttcaacgacaacaccgagtgcctggcgcgcctccacggcaagaccacctacgagaagtacctcggcccgcagtacgtcgccggcatcaccaacctcaagaagtgctccacctcccccctcctggaggcgtgcgagttcctccgcaag

SEQ ID NO: 4 - protein, 692 aa, Artificial Sequence; identical to SEQ ID NO: 2
  Recombinant sequence generated in laboratory

SEQ ID NO: 5 - DNA, 14647 bp, Bos taurus
  misc_feature (1123)..(1222): n is a, c, g, or t
  misc_feature (1498)..(1597): n is a, c, g, or t
  misc_feature (5349)..(5448): n is a, c, g, or t
  misc_feature (7480)..(7579): n is a, c, g, or t
  misc_feature (9184)..(9283): n is a, c, g, or t
  misc_feature (10656)..(10755): n is a, c, g, or t
  misc_feature (12194)..(12293): n is a, c, g, or t
  misc_feature (12987)..(13086): n is a, c, g, or t
  misc_feature (13696)..(13795): n is a, c, g, or t
  misc_feature (14137)..(14236): n is a, c, g, or t
gatcttagttcccccaaatgggtctgaacctacacatgctgcaatggaagcgccagggaagtcctcccccaccccttgggggacacttagtttgcttgcaatcagtgaacgataagcagggctgcactggagacccctgcgtgggagttgttgtgcttcaagggagtgtcttcaaggatgcagagcagagttctagcttagaactgaaaaccagcctcctgaaacagggtcagcctgtgtactgaggacaaaataggacatttatcaaaatgaggttcctgtctcccacctcatattgccacaaaacaacacaaggggtaggatatccttttcattggcaaatgagggaccaggagacagcctttgggcacttaggcctctggttctgttttctgggagctgtatttcggtctcaggaggactccaggggcagtctgggtcagactctgggcagcctctgccagctggaccaggctgccgtggaccccgggccaggcagcgggccctctttcaaaactccaggctggctctgcgtgcagatgcaagggtctccgtctgtcttaactggttcccaagcactttagataccttctctatagtcaagctgatccgcaaagattcaccctaggacccctgctctggatcccgctctctaggaggcactgagaccggagcggggacaaaacccagggactgccactcccgaagggctgcggacaagtgggaaagaaagagcaccccccaactaggcagcgctggggaacttgagaggtgggtgtgggttgggtatcctctccccgagcgccaagccccgcccgggcacctttctcgctccctcggtctccacccccgctcttcccccttccccccggttttccccctctaggaaccagcagacctcgggagaggggaggagggaggctggggcgcttataggaccacagggcggggcaaacctcgtgaggtcaccgagcactggctaaagggacgcagaacgagcgcaggtggcagagccttcgttccggagtcgccccaggacgccagccatgaagctcttcgtccccgccctgctgtccctcggagcccttggtgagtgcaggtatgagtgggggcggggcatgcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncaagaccgtctcttccagggacagtgtagaagccctcactctttggcctctttactcccaggactgtgtctggctgccccgaggaaaaacgttcgatggtgtaccatctcccaacccgagtggttcaaatgccgccgatggcagtggaggatgaagaagctgggtgctccctctatcacctgtgtgaggagggcctttgccttggcatgtatccgggccatcgcggtgagtccaggccgtaggctgggtgggaccagactgaaagggaagggatcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagtcactgcctggcataggtagtttctcctgaagctggtctggcctgtgtttcagagaaaaaggcggatgctgtgaccctggatggtggcatggtgtttgaggcgggccgggacccctacaaactgcggccagtagcagcagagatctatgggacgaaagagtgtgagttctccctggggacccagaagttgaggtggcctggacctgggccctgggctctgtggggtcagagtggacaaggcacagagctgaggtctcagacagtgtcacatgtccagggaatgatgtccctgggctctttgggccagtcaccaacccactggggaagcagaggaggcgctgggctgcagtgaccagagaattcccctcccactaggggctcattcagggagggctgtttctctctctctctggttcacgctaagagctctctattgtctctgcctcttctgcagctccccaaacccactattatgctgtggccgtcgtgaagaagggcagcaactttcagctggaccagctgcaaggccggaagtcctgccatacgggccttggcaggtccgctgggtgggtcatccctatgggaatccttcgcccgtacttgagctggacagagtcactcgagcccctccagggaggtaagacacgtcggggttctggtgatcccaggctggggtctctactccggtgtcactttcacaagccacttcggggtgtcccaggtgagcatgtggggtgaatccaaggcaggggtcaggttaactgctgtgaagcctgtggggatggggtcttctgtatcaggagccctgggagttggtagctgagtctgctgctgctgctaagtcgcttcagtcatgtccgactctgtgcgacccatagatggtgaatccctaccagcctccctcaattcatggggattttccagccaagagtacttggagtgggggggccgttcccttctccaggtagctgagtctagtttcagccactgtacatcttctgagctcctggctttccaaaggctatcaggtgggggtggaggggagggaacactgggggtgccactggctgtggtctggtgagctcacaccctggtttatttctctctgttcaacagctgtggctaaattcttctctgccagctgtgttccctgcattgatagacaagcataccccaacctgtgtcaactgtgcaagggggagggggagaaccagtgtgcctgctcctcccgggaaccatacttcggttattctggtgccttcaagtgtaaggtgagactgtcctcttctccccagcggcccatgtgtccttgggctaggaggtccctttctctgggcccaaatagagctcagtgagcatgtggggaccatggaggaggtccagttctcctgtgcagagaaactgagtatgaaaaatgtgcgggctctgtccagtgcacgatctcctccagtcccctagccccccgcagggccccctcacctctgcaccagcccagcaaacagttacctcgtcagcgtcccctcatgtctccccagagatccctgaatctggctttgtccctctgcggtcctgggctcctcctgctgacaggtgtcccctccctaactacagagggactgaaggagtcttgtcatgagcagaccagccccagacccctggccaggcagtcggacagcctggtccctctccctgggaggtagtgatctctgctcactggggagctctctccttcgccccagcctcgcactgaagctgatgtttctccttttaccccatctgtctctcggactcaggcattttgtgtgtgggggggaggcctccccgattctctgctgctcagggcacgtttaccacttagccctgggaggggaaaaaaccacgtggccgcatgtactgcttttaccctctggtgcagaggcttggaaactcttttacagagaaagacaaagttttgccctctcaatcaacccttaaaggggaaagtggggggtctctagccagcatcctctggttctttcttcctcagaatccatctgcaggtggatctattccctccatgggggccccagagcctcagagacctgtcctctgcaggtcaggagcccacagaagacaagccccagggcccctttccacaggcaggctcgaggaacaagccgggctggctgctctgtgctgacccttctggtgctttctcatcccacaggtgtctgcaggacggggctggagacgtggcttttgttaaagagacgacagtgtttggtaagtggaggagagaagccacgggtactgcctcctttgctttgctttcctttcatttcattgtaaatccaagtagtgagctaatttgactcctcacttcctggcacacgattatcaaagctctctgtagagcacagctcttatttttatgtgactttatattttaagcaatcagttttctttgaaactcccgtgcccgctatctttctcttctgcatcaaagacgcccatgtcaaggtatctgacacatgtcctccgagacagatgtagtcttgcaaagtgtatggggctgatctgtgtttctggattctaagctcacagaaagttttattgatccgttattgatgatgagtggatcaagtttcccgctcagcatcctgttttagagatttagtgttgttgtgctgtacatgtctggttcagtggttctaacactgagacgtgctctgtggagagcacacaacacatttattatccacggtcgtaaagcggtagatacgtcagtgtggtttatcttgcatttctctggcatctggtgagtttgatcatctcagtatatttgctggtagtttgggcttccctttcttcctttttcacctttgactatctttctattgattttcatgcctgtttcttcatgattttgattcaagctgaggcattcctcctctctaggtaacccgccatggttggttctagatagactttaggaaaatctcttctcagtaaaaaaaaaaaaaaaaatctgtcagtaacaggtttcttgaacagaagtccttgatattgataaagtcaaattggccactttggccctgctggcatatgtttgggggtcttcatttatttattttttaatccaatagcattgacttttattcattcatgttaagtcacctgaaatggtatctgttgcatttaaatgctcaattaatgtaaatggctgaacaaatccattttaatggatattcaatccattaaaaaaactcaatatcccacataaggtcacttaacatagcctcatgacaactcccacaccaaaacagtactttattttgtaaattttgaccattattactcccatgttatggtcttttcagctgtcaagcaaacaaggtgaagaaaaaatttagttagatgggggttgcacctggaaaataaatttcttaaactccatatacatgtttcaaatctgctgggtcccaagtccatctatgaattcccaggctgccagtatcatatgcagcatactaaagctacgctatctgaatagcttattaattctgcatatatcaggtcaaccgatgtgtcaacctgtaacagactgcgacacttttaactgaacatggcaaaatattcactctcttgaactttacatcatctttgatatcaaacaatgaggcaaatcctaacagtatatcaaaaaacaacttttgagttttacaaaagattgttcccatactgattaatcaggcaagctaactcttaggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaactctcccttccccaaagagaacttgccagagaaggctgacagggaccagtatgagcttctctgcctgaacaacagtcgggcgccagtggatgcgttcaaggagtaccacctggcccaggtcccttctcatcctgtcgtggcccgaagtgtggatgccaaggaagacttgatctggaagcttctcagaaaggcgcaggtacccccaccctcagtacccccacccgcttgggtctggtggtggggagggtttccttccttcctcctccttccctaacttcctgccactctggaatattcaagtggcctgcctgtcataggactgtactccatacagttgaaaccatgacccctcctctagcttgacccactggacaggtttctaatccgaagctggaacagaacagaataggttggagggtgatgcaatattgtcaagactctaaggtgctcagaccccaccaacttcataaagacaaagccatgactgagctatgtctcagggaggccgtgagatcctctacaggaggaggtcaacccaggttcctgctctcagaatcatcccggagttgcagatactctgtttcaacctggtgtttcagtttcttgtctaccactgacatcataattctctttcccttaattaggagaaatttggaaaaaacaagtctcggagcttccagctctttggctctccacccggccagagggacctgctgttcaaagactctgctcttgggtttttgaggatcccctcgaaggtagattcggcgctgtaccttggctcccgctacttgaccaccttgaagaacctcagggaaagtgagtgagggccaggcccaccctaagggcaggccggtgtgggtggtgagccttggctgaggccgtcggggagcacctccccaccctccacccccgcccgctacagacagcttggccctgctctgcagtcgtcccttggtcacttttccagcccctggaggtcttcaccatggtaggtgggacgcgaatgtgagcgtcttcttgtggaccaaatcggctgaccggtgtctccacaggagcacgtcgcctgctgataacgctgcgcaggaccccgtgtgcggcgggttgcccagccctggtagagcagtcagtttgcctctggggaagtgagaggtacgaagacaccccgagagagccagggcccttctgggatgaatccagagccctgggccctgaacttggccacgataccaccctgggccagctcagctgtggctcttcctctgctggaggcttcaggcggactcggggtgaacggggaggctgcactgttgttacagcctgttgaccgctgtgtccccgggcagctgcggaggaggtgaaggcgcggtacaccagggtcgtgtggtgtgccgtgggacccgaggagcagaagaagtgccagcagtggagccagcagagcggccagaacgtgacctgtgccacggcgtccaccaccgacgactgcatcgtcctggtgctggtagggggcctgctgccggcctgcaagctgtgcgggtggggggcacaggtcaccgcatctggttggaggggagcaaggggctcagacggtgctccccccccaggggggagcagggcatgctcgtcctgcctgcccagagtggggacagacagagctttctgtcctccagagtgtctactgtggacttcccactcttctgatcttatagaacaactcacaatgaatgcaggtaatctggaaaatttagaaagatgtgatgtaaaaccaaacagcaatggagcctcacaaggggctaccacagttgagtgcgtgttagtcactcagtcatgtctgaccccttgtggccccatggactgtagccctcctctgcccatggaattctccaggcaagaatactggagtgggttgccatgcccttctccaggggatcttcccgaaccagggcgttgaatccgagtctcccacatagaagacagattgtttaccatctgagccagcaggaaagcacctagcacatagttaagagttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggggggtaaaagaacccaccttccaatgcaggagacggtgggttcaatccctgggtccaagaagatcccccttggaggaggaaatggcaaaccccctccagtatccttgcctggagatcccatacacagaggagcctgtgggctacagtccatggggtcgcaaagagtcgttcacgtcttagtgactccacagcagccacgaccgggccgcctgttcccggtctttgcagtgggagtgacggcgtgaaaggcgtgcacggccggtacagaagtaatggtgcacgtggcgttgcctcgggctgctcacatcaatcctgctgcttcactgggcacctgacgtccgttctcttagagctgtaaagacaggcagggtgggaaatagcctcaccgtggtgctgaagatatgctttgtcttggcagaaaggggaagcagatgccctgaacttggatggaggatatgtctacactgcgggcaagtgtggcctggtgcctgtcctggcagagaaccggagtaagtggagaagagtaccccctggcttattccctctgggcttcgagggcggggtggagagcagtaaccccactcggtggttcatgcggagacattaaaatggacagataggttcaaacaggagaatcaggagaaagaatagatgaaagtggctgtgttacccatatggcctagtgtggtgagaactgacagatgctcctctgtcagattgatttcctctttttgatttttgaaaagattttagtaagttataatatacatactgaaaagtgcacctaagtgtacaactcaagaacatttcacacactggtgcatctccaagctctagtgcccacagcgagatacagcaggttccacaagtcctcttgcgtccaccagccctcttaccccggcaagggtaacgactcctgacgtcaaatggcacaaatcggctctgcccgtctttgagcttgatgtaaaaggagttgcacagcaggtaccttttaaaaagtcttttgctcagcaatatatctcgttttgcagtattctgttgtgactccgctgcagttacttatttattctactgtggggaagcatttgggtaatttccaatggttggctgccgtggatagagttgctgtgaacatctggatccttgtcttttggtgaacacatacatgcatttctgttgcatggaactgctgggatggtctgtttggatttgattctcgactgctgagatttaaaagtttgtgatttctcaggtggagaaagacagataccttctgatgtcactcctatgtgggatagttaaaaactaagtgaataaaccaaatcaagcaaaaacaaatacacagattccaggaacagagtggtggataagaaaacagagcaagagatgtgggggtagagggaaatgtggtaatgggggtaggtgatggtatggaaactaaacttttggtggtcagcaactgtcaggtaaggggcttccccagtgggcagtgataaagaatacgcctgcgatgcgagagagtcaagagacatgggctcgatccctgggttaggagatcccctgaaggaggaaatgacaacccactccagtattcttgcctggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttgacctagccccaacagaaagagactcacagacatgctgagcacactggtacccaaggcgggtgggaccagggagagccacagactgggagttcgggcttggtagatgcaagctgttacttttagaacggataaacggcaagcccctgctgtacagtacaggaaactgtgtctggtttcctggataaaccataatggaagcaaatataaaaagacaaatgtgtatatgtgtttaactgagtcactttgccatacggcaaagatcgacacgacactgtcaatcaactacacgtcagtttaaaaaaagaacttaaaaacacagcgttgggttgaactgaaaacaaaaaccagaaaaggaagaaacacagtgaagtctattctacaatgccatttatttatgttgaaaacaaacgtatacaaagccatgccaatatgcatttttcaatatgtgggtacctttggaaaataaatgaacacactaaacgatgtgtttaatgacattttgcttctctttattctttcagaatcctccaaacacagtagcctagattgtgtgctgagaccaacggaaggtgagttcagggtcaagggtacattcttgtccctggaggaggagaggttgggaacttaaaaaaaaaaaactcacagagatgcagccatagaagctgtgagttctttgcagatgccagtcattcagagatgagagtgctgggattcacgtcacatggaccctcacacatggcagagaaatcttagagatcctcactgtgtcctgagcacggggaaggaggggtgagccatccccatctcgggcgggatgaagaaacagacccctagatctcactcatagcccatggataatactgtgcagatcctgcccggaaccagggtcccttgtgctaaatccaatgcgctgttcaaaagccaagccacaccaccctaaagagctggtgccctccctcggcttggggtaaaagagtgacatttgccgacaaaaagactctaccagttttgacctaccaggaggggatctccaccccacttaagcttaagctgtgttggtctatttgacactgaccagtcatctggggggatcgttgcctagcgatgcttcttaacctttgtgggcctaggttgcactctgtgcaatagagtccatggaccagcccctgggaggttgacttgctcttccgtttgaccaagggtaccttgccgtggcagttgtcaggaaagcaaatgaggggctcacatggaattctctgaaagacaagaagtcgtgccacaccgccgtggacaggactgcaggctggaacatccccatgggcctgatcgtcaaccagacaggctcctgcgcatttggtaggagtcctgannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggtaccaggtgccctggtaaaaagcagggttcctattactgaggaaaaggggagagcacaacctgaacattttggagcttctgatacaatggccatggagttcatcaggctgagtcccctgctctaggctgcgaggatggaaccctttgtacagtcatagaccctcttctccagccccagtcaggttgcctatagtctcaagttttgctgaatcctgttggtgcacaggttggtggtaacttgttactatgtgttataatgactgtgttctgtctggccttggtcagaccaagccacatctgtcacctgttaggcagagaccacttaatcttaacacaggtaggaagagctggctccccatgtttcttgctctgcagatgaattctttagtcagagctgtgcccctggggctgacccgaaatccagactctgtgccttgtgtgctggcgatgaccagggcctggacaagtgtgtgcccaactctaaggagaagtactatggctataccggggctttcaggtgagtgttgtggccaggcttcatcaggacagggccctggccctcagcctccaggatcttggcacatcacatttcattttttttttcctctttgaaaaatatatttcccttcaattttgaattgggaggatgactgcttaacaatgttgtgttggcctcctgcaaaagaaaatcacccgtaagtaccagaagtaaaacgcacttggcctccttcgctcccccctgccccgtggaacaccgctgtatatcgctgagcaccaagttgagctccctgcctggtacagcgactctctgtttcacacgtggtgatgtttacatttcagagcttctctctcagttcgtcccaccctctccttcgccctgctgtgcccgcaagtctttggcactgcattgctaaggggcccgatgtggtctgtcgggggcagcctggggatcagctgatgggcgggctggggagggctcctcccacaacacccaccatcagactgaccccggggctgtctggacctcagctctgctggcaccccttcctgtgtcctggaaccaaggttgatctcagtggacctcagtggcatgttcagaatcagagagggagaggttgaggtataagtcctttcgggaactcagttttatggggaagattctctttcccgtaggcgccaccttctcccgcagcctaggtagttatgtctcacgtttttctgctctctttgattttggccttagaaatggcagagagagagagtgttgcagagttattctccaaggcaatggcaccccactccagtactcctgcctgggaaaatcccatggatggagggagcctggtgggctgcagtccatggggtcgctaagagtcaaggcatgacttagtgacttcactttcacttttcactttcatgcatttggagaaggaattnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatattttgtctttcttttttctatttgcttccatgtttcagtttttggaatgtgtattataaatagctatgaaaattctaatagtgtttacctctctggcaatgttttgattctctggaacaatttaggtgcctggctgaggacgttggggacgttgcctttgtgaaaaacgacacagtctgggagaacacgaatgggtaggtaaaggtgttttcctttcccccttcaagagcacgatccttgccgtcaccaacagagcctactgttgtggcagtcccagcgcgggttggaaaagaatttcctgacacaaagtatttcagcaaggagtgactttataagaacaaggagcagagataacgagggtctgcaggcacagcgggccgacctcctgaccagggatagttgaccaaatgctgttggaacagcaactaggctcaagggagaactgactctttcctgggttagtagccaacttttataacctcgagacgaaaattgctgctggaagggtggcattaggtgattggttagggcgctagagagtgagtaccggggtaggcattttttcctgttaataatacggggtcaggaaactggtcagtaaggatcaggtgggcttttggcaatggttgcaatggggctcagtcagttccagaggtgaccttgattctgagccacagaagcacactatctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnccaccaatcactttctgtcttacctattgtgggcatatgacagaaatggaatcctatgataagtggtcttctgtgattggcttgtcttattttgcatcatggttttcatccggcgtggggtagcatgtgttagaacttgacttcttcttcttgttgctgagtaagactccatcgtggagcactggtatttgatacgtttgaagtctgtataacttgaggagctttctttgttctccttcacgtgtgtgcatttgaagagggccggtgggtggtgacagccccagaaaggaggctttgactcagccgggcagccgtggctgatgcagccttctctctggtctccagagagagcactgcagactgggctaagaacttgaatcgtgaggacttcaggttgctctgcctcgatggcaccaggaagcctgtgacggaggctcagagctgccacctggcggtggccccgaatcacgctgtggtgtctcggagcgatagggcagcacacgtgaaacaggtgctgctccaccagcaggtaaggatcacagggcctcctgcctgtgcttaccttcaggggctcccctcaggtgagcaaccacagagctcagcggatccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctgatcctgcacagcagagattgcctcccaagtccataatctctcctcttttcctcagctggtctcttactgtctgcccttctgacataggctctgtttgggaaaaatggaaaaaactgcccggacaagttttgtttgttcaaatctgaaaccaaaaaccttctgttcaatgacaacactgagtgtctggccaaacttggaggcagaccaacgtatgaagaatatttggggacagagtatgtcacggccattgccaacctgaaaaaatgctcaacctcccgtaagttggaatataaataagatcactgagaaagcgtctgcatgaaggtcaaagttggaggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaagcttgacctacagtggggaagaagggcagagggaagtctgttttagtgtcttctttgattgatgtattgattccaagaaaaaaaaaaaaggaatccttttctgaatctctggcttcgttgtgtcccacagcgcttctggaagcctgcgccttcctgacgaggtaaagcctgcaaagaagctagcctgcctccctgggcctcagctcctcccgtctctcagccccaatctccaggcgcgagggaccttcctctcccttcctgaagtcggattttgccaagctcatcagtatttacaattccctgctgtcattttagcaagaaataaaattagaaatgctgttgattttcattccctataaaatgccaattatcttctctcaaatgtatactgaaatcgtgtctggaccaa

SEQ ID NO: 6 - protein, 708 aa, Bos taurus
MKLFVPALLSLGALGLCLAAPRKNVRWCTISQPEWFKCRRWQWRMKKLGAPSITCVRRAFALACIRAIAEKKADAVTLDGGMVFEAGRDPYKLRPVAAEIYGTKESPQTHYYAVAVVKKGSNFQLDQLQGRKSCHTGLGRSAGWVIPMGILRPYLSWTESLEPLQGAVAKFFSASCVPCIDRQAYPNLCQLCKGEGENQCACSSREPYFGYSGAFKCLQDGAGDVAFVKETTVFENLPEKADRDQYELLCLNNSRAPVDAFKEYHLAQVPSHPVVARSVDAKEDLIWKLLRKAQEKFGKNKSRSFQLFGSPPGQRDLLFKDSALGFLRIPSKVDSALYLGSRYLTTLKNLRETAEEVKARYTRVVWCAVGPEEQKKCQQWSQQSGQNVTCATASTTDDCIVLVLKGEADALNLDGGYVYTAGKCGLVPVLAENRKSSKHSSLDCVLRPTEGYLAVAVVRKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLNREDFRLLCLDGTRKPVTEAQSCHLAVAPNHAVVSRSDRAAHVKQVLLHQQALFGKNGKNCPDKFCLFKSETKNLLFNDNTECLAKLGGRPTYEEYLGTEYVTAIANLKKCSTSPLLEACAFLTR

SEQ ID NO: 7 - DNA, 2133 bp, Homo sapiens
catgaaacttgtcttcctcgtcctgctgttcctcggggccctcggactgtgtctggctggccgtaggagagggagtgttcagtggtgcgccgtatcccaacccgaggccacaaaatgcttccaatggcaaaggaatatgagaagagtgcgtggccctcctgtcagctgcataaagagagactcccccatccagtgtatccaggccattgcggaaaacagggccgatgctgtgacccttgatggtggtttcatatacgaggcaggcctggccccctacaaactgcgacctgtagcggcggaagtctacgggaccgaaagacagccacgaactcactattatgccgtggctgtggtgaagaagggcggcagctttcagctgaacgaactgcaaggtctgaagtcctgccacacaggccttcgcaggaccgctggatggaatgtccctatagggacacttcgtccattcttgaattggacgggtccacctgagcccattgaggcagctgtggccaggttcttctcagccagctgtgttcccggtgcagataaaggacagttccccaacctgtgtcgcctgtgtgcggggacaggggaaaacaaatgtgccttctcctcccaggaaccgtacttcagctactctggtgccttcaagtgtctgagagacggggctggagacgtggcttttatcagagagagcacagtgtttgaggacctgtcagacgaggctgaaagggacgagtatgagttactctgcccagacaacactcggaagccagtggacaagttcaaagactgccatctggcccgggtcccttctcatgccgttgtggcacgaagtgtgaatggcaaggaggatgccatctggaatcttctccgccaggcacaggaaaagtttggaaaggacaagtcaccgaaattccagctctttggctcccctagtgggcagaaagatctgctgttcaaggactctgccattgggtcttcgagggtgcccccgaggatagattctgggctgtaccttggctccggctacttcactgccatccagaacttgaggaaaagtgaggaggaagtggctgcccggcgtgcgcgggttgtgtggtgtgcggtgggcgagcaggagctgcgcaagtgtaaccagtggagtggcttgagcgaaggcagcgtgacctgctcctcggcctccaccacagaggactgcattgccctgaaaggagaagctgatgccatgagtttggatggagggtatgtgtacactgcaggcaaatgtggtttggtgcctgtcctggcagagaactacaaatcccaacaaagcagtgaccctgatcctaactgtgtggatagacctgtggaaggatatcttgctgtggcggtggttaggagatcagacactagccttacctggaactctgtgaaaggcaagaagtcctgccacaccgccgtggacaggactgcaggctggaatatccccatgggcctgctcttcaaccagacgggctcctgcaaatttgatgaatatttcagtcaaagctgtgcccctgggtctgacccgagatctaatctctgtgctctgtgtattggcgacgagcagggtgagaataagtgcgtgcccaacagcaatgagagatactacggctacactggggctttccggtgcctggctgagaatgctggagacgttgcatttgtgaaagatgtcactgtcttgcagaacactgatggaaataacaatgacgcatgggctaaggatttgaagctggcagactttgcgctgctgtgcctcgatggcaaacggaagcctgtgactgaggctagaagctgccatcttgccatggccccgaatcatgccgtggtgtctcggatggataaggtggaacgcctgaaacaggtgttgctccaccaacaggctaaatttgggagaaatggatctgactgcccggacaagttttgcttattccagtctgaaaccaaaaaccttctgttcaatgacaacactgagtgtctggccagactccatggcaaaacaacatatgaaaaatatttgggaccacagtatgtcgcaggcattactaatctgaaaaagtgctcaacctcccccctcctggaagcctgtgaattcctcaggaagtaaaa

SEQ ID NO: 8 - protein, 12 aa, Homo sapiens
EDCIALKGEADA

SEQ ID NO: 9 - protein, 709 aa, Homo sapiens [sequence omitted]

(19) United States
(12) Patent Application Publication
(10) Pub. No.: US 2021/0023179 A1
(43) Pub. Date: Jan. 28, 2021

(54) LACTOFERRIN COMPOSITIONS AND METHODS FOR MODULATION OF T CELL SUBTYPES AND TREATMENT OF AUTOIMMUNE DISEASES

(71) Applicants: Ventria Bioscience Inc., Junction City, KS (US); The Regents of the University of Colorado, a body corporate, Denver, CO (US)

(72) Inventors: Christopher MacManus, Fort Collins, CO (US); Colm Collins, Denver, CO (US); Edwin De Zoeten, Denver, CO (US)

(21) Appl. No.: 16/998,349

(22) Filed: Aug. 20, 2020

Related U.S. Application Data
(63) Continuation of application No. 15/758,537, filed on Mar. 8, 2018, now abandoned.
(60) Provisional application No. 62/217,782, filed on Sep. 11, 2015.

(51) Int. Cl. A61K 38/40; A61P 37/02; A61P 1/00; A61P 29/00

(52) U.S. Cl. CPC A61K 38/40; A61P 29/00; A61P 1/00; A61P 37/02

(57) ABSTRACT

Provided herein are improved compositions comprising plant-derived recombinant human lactoferrin (rhLF) and treatment methods for modulating an immune response to improve the balance between anti-inflammatory cytokine producing cells and pro-inflammatory cells, and skew naïve T cells toward a pro-regulatory phenotype.

### CROSS-REFERENCE TO RELATED APPLICATIONS

This application is a continuation of U.S. Ser. No. 15/758,537, filed 8 Mar. 2018, as the U.S. National Stage of International Patent Application No. PCT/US2016/051384, filed 12 Sep. 2016, which claims priority to U.S. Provisional Patent Application Ser. No. 62/217,782, filed 11 Sep. 2015, the disclosures of which are each herein incorporated by reference in its entirety.

### STATEMENT REGARDING FEDERALLY SPONSORED RESEARCH

This invention was made with Government support under Grant number 11618673 awarded by the National Institute of Health. The Government has certain rights in the invention.

### SEQUENCE LISTING

The instant application includes a sequence listing named “SequenceListing, submitted electronically via EFS in the form of a text file created 8 Sep. 2016, which is incorporated into the application by reference in its entirety.

### TECHNICAL FIELD

The present disclosure relates to the field of therapeutic methods and compositions for treatment of immune-related disorders in mammalian subjects. More particularly, the methods and the compositions herein described comprise lactoferrin (LF), and are directed to modulation of certain T cell populations and/or phenotypes and their activities in a subject having a neurodegenerative or autoimmune disease, to improve the balance between anti-inflammatory (Th2) cytokine producing cells and pro-inflammatory (Th1) or Th17 cells, and to skew naïve T cells toward a pro-regulatory phenotype for treatment, amelioration or prevention of immune related diseases and disorders (e.g., inflammatory bowel disease (IBD), amyelotrophic lateral sclerosis (ALS) and/or rheumatoid arthritis (RA)).

All patents, patent applications, patent publications, scientific articles and the like, cited or identified in this application, are hereby incorporated by reference in their entirety in order to describe more fully the state of the art to which the present application pertains.

### BACKGROUND

Autoimmune disease arises from a dysregulated immune response toward a self-antigen. This aberrant activation of an immune response results in an overproduction of pro-inflammatory cytokines such as TNF by monocytes, macrophages, and T cells, thereby allowing for persistence of a hyperactivated immune response and subsequent pathology (Naser, et al., 2011, Clin. Vaccine Immunol. 18:1416-9; Van Deventer, S. J., 1997, Gut 40:443-8). The contribution of CD4⁺ T cells has been identified as being an important driver of pathogenesis in numerous autoimmune conditions, whereby an increase in activation and populations of CD4⁺ and CD8⁺ T cells is observed (Funderburg, et al., 2013, Immunology 140:87-97). An over-activation of Th1/Th17 phenotypes has also been found to drive much of the chronic pathology in autoimmunity (Zenewicz, et al., 2009, Trends Mol. Med. 15:199-207).

The activity of pro-inflammatory T cells also can be tempered by the action of a subset of CD4⁺ regulatory T cells (Treg). Regulatory T cells were first identified by their elevated expression of the high-affinity IL-2 receptor CD25 (IL-2Ra). Classically defined Tregs are found within the CD4⁺ T-cell pool and are identified by their constitutive expression of Foxp3, and the IL-2 receptor α-chain (CD25) (Rudensky A. Y., 2011, Immunol. Rev. 241:260-8). Mice lacking IL-2 signaling via antibody neutralization or genetic deficiency of IL-2 or IL-2 receptors show natural Treg (nTreg) deficiencies and spontaneous autoimmune disease including inflammatory bowel disease (IBD). Pioneering studies by Powrie et al. (Powrie, et al., 1993, Int. Immunol. 5:1461-71; Powrie, et al., 1994, J. Exp. Med. 179:589-600) demonstrated that the pathology in a mouse model of T-cell-induced colitis, which mimics human IBD, can indeed be prevented by adoptive transfer of Foxp3⁺ Tregs. Furthermore, Tregs can not only prevent but also cure IBD in mouse models (Mottet, et al., 2003, 1 Immunol. 170:3939-43).

CD4⁺Foxp3⁺ T regulatory (“Treg” or “Treg”) cells control many facets of immune responses ranging from autoimmune diseases, to inflammatory conditions, and cancer in an attempt to maintain immune homeostasis. Natural Treg (nTreg) cells develop in the thymus and constitute a key arm in the system of peripheral tolerance particularly to self antigens. A growing body of knowledge now supports the existence of induced Treg (iTreg) cells which may derive from a population of conventional CD4⁺ T cells. The fork-head transcription factor (Foxp3) typically is expressed by natural CD4⁺ Treg cells, and thus serves as a marker to definitively identify these cells. There is less consensus on what constitutes iTreg cells as their precise definition has been somewhat elusive. This is in part due to their distinct phenotypes which are shaped by exposure to certain inflammatory or “assault” signals stemming from the underlying immune disorder. The “policing” activity of Treg cells tends to be uni-directional in several pathological conditions. On one end of the spectrum, Treg cell suppressive activity is beneficial by curtailing T cell response against self-antigens and allergens thus preventing autoimmune diseases and allergies. On the other end however, their inhibitory roles in limiting immune response against pseudo-self antigens as in tumors often culminates into negative outcomes. With regard to this latter aspect of Treg cell immunobiology, nTreg cells are now believed to be involved in various animal models and human tumors. Of additional interest are iTreg cells, their relationship with their natural counterpart, and potential co-operation between the two in modulating immune response against tumors. Several studies have focused on these cells as targets for improving anti-tumor immunity (Adeegbe and Nishikawa, 2013, Front. Immunolog. 4:190).

T cells can be genetically modified to target tumors through the expression of a chimeric antigen receptor (CAR). Most notably, CAR T cells have demonstrated clinical efficacy in hematologic malignancies with more modest responses when targeting solid tumors (Bonifant, et al., 2016, Molecular Therapy—Oncolytics 3, Article number: 16011).

A sustained neuroinflammatory response is the hallmark of many neurodegenerative diseases, including Parkinson's disease, Alzheimer's disease, cognitive decline in the elderly resulting from chronic inflammation, amyotrophic lateral sclerosis (ALS), multiple sclerosis, and HIV-associated neurodegeneration.

FOXP3⁺ CD25⁺ CD4⁺ regulatory T cells (Tregs), are pivotal in suppressing autoimmunity and maintaining immune homeostasis by mediating self-tolerance at the periphery as shown in autoimmune diseases and cancers. A growing body of evidence shows that Tregs are not only important for maintaining immune balance at the periphery but also contribute to self-tolerance and immune privilege in the central nervous system. Evidence supports a dysfunction of Tregs in several neurodegenerative diseases. In some cases, dysfunction of Tregs is observed in the early stages of several neurodegenerative diseases, but not in their chronic stages, pointing to a causative role of inflammation in the pathogenesis of neurodegenerative diseases. A number of molecules, such as hormones, neuropeptides, neurotransmitters, or ion channels, affect the dysfunction of Tregs in neurodegenerative diseases. The intestinal microbiome also plays a role in the induction and function of Tregs, and greater study of the crosstalk between the enteric nervous system and Tregs in neurodegenerative diseases is needed (He, F., and Balling, R., Wiley Interdiscip. Rev. Syst. Biol. Med. 2013 March-April; 5(2): 153-80).

Recently, it was reported that T cells from patients with ALS can be used to generate Tregs for adoptive cell therapy. ALS is a progressive neurodegenerative disorder affecting upper and lower motor neurons, and there is compelling evidence for a neuroprotective role for Tregs in this disease. For example, rapid progression in ALS patients is associated with decreased FoxP3 expression and Treg frequencies. Restoration of Treg number and function may slow disease progression in ALS; thus, a procedure was developed to enrich and expand in vitro Tregs from ALS patients. Tregs isolated from these patients were phenotypically similar to those from healthy individuals but were impaired in their ability to suppress T-cell effector function. In vitro expansion of Tregs for 4 weeks in the presence of GMP-grade anti-CD3/CD28 beads, interleukin (IL)-2 and rapamcyin resulted in a 25- to 200-fold increase in their number and restored their immunoregulatory activity (Alsuliman, et al., Cytotherapy, August 2016, in press).

Underscoring the important role of Tregs in immune regulation, Foxp3 deficiency results in global failure of Treg cell development, leading to a lethal multi-system autoimmune disease immunodysregulation polyendocrinopathy enteropathy X-linked syndrome (IPEX) (Yong, et al., 2008, J. Clin. Immunol. 28:581-7; Carneiro-Sampaio, M. and Coutinho, A. 2015, Front. Immunol. 6:185). The most commonly affected organ in IPEX is the intestine, highlighting an important role for Treg cells in the gastrointestinal tract. In addition to IPEX, other genetic deficiencies suggest the involvement of Treg in IBD. Mutations in WAS protein (WASP), CD25, and IL-10 all lead to abnormal Treg cell numbers and/or function, and also increase an individual's risk for autoimmune disease (Boden, E. K. and Snapper, S. B. 2008, Curr. Opin. Gastroenterol. 24:733-41).

Lactoferrin (LF) is an iron-binding glycoprotein of the transferrin family, which is expressed in most biological fluids with particularly high levels in mammalian milk. Its multiple activities lie in its capacity to bind iron and to interact with the molecular and cellular components of hosts and pathogens. LF can bind and sequester lipopolysaccharides (LPS), thus preventing pro-inflammatory pathway activation, sepsis and tissue damages. LF is also considered a cell-secreted mediator that bridges the innate and adaptive immune responses. In the recent years much has been learned about the mechanisms by which LF exerts its activities. (Siqueiros-Cendon, et al., 2014, Acta Pharmacol. Sin. 35(5):557-66). Lactoferrin (LF) is a single-chain iron-binding glycoprotein of approximately 80 kDa that belongs to the human family of transferrins (Brock J. H. 2002, Biochem. Cell Biol. 80:1-6). LF is present in myriad mucosal fluids (Laibe, et al., 2003, Clin. Chem. Lab. Med. 41:134-8; Ohashi, et al., 2003, Am. J. Ophthalmol. 136:291-9; Niemela, et al., 1989, Hum. Reprod. 4:99-101; Lin, et al., 2001, Oral Microbiol. Immunol. 16:270-8; Caccavo, et al., 1999, Int. J. Clin. Lab. Res. 29:30-5), but is most predominant in human milk, particularly in the colostrum during early lactation, where it has been suggested to promote the healthy growth and development of the GI tract (Zhang, et al., 2001, Adv. Exp. Med. Biol. 501:107-13), promote the growth of commensal bacterial populations and protect against the establishment of pathogenic bacteria and viruses (Barboza, et al., 2012, Mol. Cell. Proteomics 11:M111 015248; Ochoa, T. J. and Cleary, T. G., 2009, Biochimie 91:30-4; Ammendolia, et al., 2012, Pathog. Glob. Health 106:12-9). Human colostrums and mature breast milk contain 5.8 mg/mL and 3.3 mg/mL of LF, respectively (Montagne, et al., 1999, J. Pediatr. Gastroenterol. Nutr. 29:75-80; Montagne, et al., 2001, Adv. Exp. Med. Biol. 501:241-7). In contrast, bovine colostrum and milk contain markedly reduced concentrations of LF (1.5 mg/mL in colostral whey and 20-200 μg/mL in milk) (Steijns, et al., 2000, Br. J. Nutr., 84 Suppl. 1:S11-7). LF has been previously identified for its multifactorial and beneficial activities in several models of human health including inflammation (Mueller, et al., 2011, Curr. Med. Res. Opin. 27:793-7; Zavaleta, et al., 2007, J. Pediatr. Gastroenterol. Nutr. 44:258-64), wound healing (Lyons, et al., 2007, Am. J. Surg. 193:49-54), infectious diseases (Zavaleta, et al., 2007, J. Pediatr. Gastroenterol. Nutr. 44:258-64; King, et al., 2007, J. Pediatr. Gastroenterol. Nutr. 44:245-51; Ochoa, et al., 2008, Clin. Infect. Dis. 46: 1881-3) and cancer (Parikh, et al., 2011, J. Clin. Oncol. 29:4129-36; Hayes, et al., 2010, Invest. New Drugs 28:156-62).

Oral administration of human lactoferrin (hLF) has been shown to suppress a number of pro-inflammatory cytokines in numerous models of colitis and sepsis, including TNF, IL-1β and IL-12. In other studies of mucosal inflammation in mice, hLF has been shown to be more efficacious than bovine lactoferrin (bLF) (Haversen, et al. 2000, Infect. Immun. 68:5816-23). In experimental models of sepsis and rheumatoid arthritis, LF has been demonstrated to exert protection through inhibiting the production of pro-inflammatory cytokines (TNF, IL-6 and IL-1β) and stimulating anti-inflammatory and pro-restitution cytokines (IL-10 and IL-4) (Kimber, et al., 2002, Biochem. Cell. Biol. 80:103-7; Togawa, et al., 2002, Am. J. Physiol. Gastrointest. Liver Physiol. 283:G187-95; Togawa, et al., 2002, J. Gastroenterol. Hepatol. 17:1291-8; Haversen et al., 2003, Scand. J. Immunol. 57:2-10; Hayashida et al., 2004, J. Vet. Med. Sci. 66:149-54; Machnicki et al., 1993, Int. J. Exp. Pathol. 74:433-9). The molecular mechanisms through which LF exerts its anti-inflammatory effects is not completely understood, but in part appear to occur through the inhibition of nuclear factor kappa B (NFκB) signaling pathways, through inhibition of intracellular TNF receptor associated factor 6 (TRAF6) signaling (Inubushi, et al., 2012, J. Biol. Chem. 287:23527-36). Further studies have demonstrated the ability for LF to enter the nucleus of the cell where it directly interacts with the NFκB response elements of pro-inflammatory genes thereby preventing NFκB induced gene expression in human monocytic and endothelial cells (Haversen et al., 2002, Cell. Immunol. 220:83-95; Kim, et al., 2012, FEBS Lett. 586:229-34). Interestingly, in a study investigating the role of human LF in the monocytic leukemia cell line, THP-1, it was shown that human LF demonstrates moderate activation of NFκB in a TLR4-dependent mechanism through the action of the carbohydrate moieties decorating LF, whereby the protein backbone of LF inhibits LPS-mediated activation of TLR4 (Ando, et al., 2010, FEBS J. 277:2051-66).

Further evidence for the anti-inflammatory effects of LF have come with the observation that a 20 amino acid peptide derived from the N terminal of LF (LFP-20) inhibit LPS-induced MyD88/NF-κB and MyD88/MAPK signaling independent of direct interaction with LPS (Zong, et al., 2015, Dev. Comp. Immunol. 52:123-131). It has also been reported that bovine LF acts as a potent anti-inflammatory agent on monocytes by triggering a tolerogenic-like program during their differentiation into dendritic cells (DC) (Puddu et al., 2011, PLoS One 6:e22504). In a recent study by Akin et al., oral adminiatration of bovine LF at 200 mg per day was shown to reduce the incidence of necrotizing enterocolitis (NEC) in preterm infants. Their studies reported fewer sepsis episodes in the bovine LF intervention group (4.4 vs. 17.3/1,000 patient days, p=0.007) with none developing NEC, though without statistical significance (Akin, et al., 2014, Am. J. Perinatol. 31:1111-20; See also Clinical Trial NCT01287507). LF was known to have antibacterial functions, and, in the aforementioned study, Akin was evaluating the possible effect of LF on sepsis a (bacterial pathogenic infection), rather than identifying any effect LF had on immune dysregulation or Treg involvement inflammation.

A recent study has also demonstrated that LF coordinates with TGF-β to efficiently direct the differentiation of Treg from naïve T cells, with potential for use in Treg cell therapy for transplant rejection (Pyeung-Hyeun Kim S-JK, et al., 2016, Journal of Immunology, 196:133.24).

A product known as Neolactoferrin, a combination of recombinant human lactoferrin (90%) and goat lactoferrin (10%) isolated from the milk of transgenic goats carrying the full-length human lactoferrin gene, was reported to enhance production of IL-1β in vitro. Specifically, iron-saturated Neolactoferrin was reported to increase synthesis of pro-inflammatory cytokine TNFα, which then determined the direction of the differentiation of precursor dendrite cells. Under the action of T cells, Neolactoferrin was reported to amplify the expression of the transcription factors responsible for the differentiation of Th- and Treg-cells and stimulated the production of both IFNγ and IL-4. These researchers reported that Neolactoferrin exhibits an immunotropic activity and hinders the development of immune inflammatory processes. In contrast to the present disclosure using lactoferrin compositions, the pro-inflammatory activity of Neolactoferrin was dependent on the state of iron saturation, and no significant effect was observed on the expression of “pro-inflammatory” gene TBX21 (encoding the Tbet factor of Th1 cells) or RORC (encoding the RORc factor of Th17 cells) (Chernousov, et al., 2013, Acta Naturae. 5(4):71-77).

Wiskott-Aldrich syndrome (WAS) is a rare X-linked recessive immunodeficiency characterized by eczema, thrombocytopenia, bloody diarrhea, immune deficiency and recurrent infections; WAS patients often develop autoimmunity and allergy. The WASp gene codes for the WAS protein (WASP), 502 amino acids in length, and is mainly expressed in hematopoietic cells. WASP activates actin polymerization by binding to the Arp2/3 complex. In T-cells, WASP is activated via T-cell receptor signaling pathways to induce cortical actin cytoskeleton rearrangements that are responsible for forming the immunological synapse. CD4⁺CD25⁺FOXP3⁺ natural regulatory T (nTreg) cells have a role in peripheral tolerance to prevent immune responses to self-antigens and allergens, and the effect of WASP deficiency on the distribution and suppressor function of nTreg cells has been investigated. WAS^{-/-} nTreg cells engrafted poorly in immunized mice, indicating perturbed homeostasis, and WAS^{-/-} nTreg cells failed to proliferate and to produce transforming growth factor β upon T cell receptor (TCR)/CD28 triggering. Compared with WT nTreg cells, WAS nTreg cells showed reduced in vitro suppressor activity on both WT and WAS effector T cells. Similarly, peripheral nTreg cells were present at normal levels in WAS patients but failed to suppress proliferation of autologous and allogeneic CD4⁺ effector T cells in vitro. Overall, WASP appears to play a significant role in the activation and suppressor function of nTreg cells, and a dysfunction or incorrect localization of nTreg cells may contribute to the development of autoimmunity in WAS patients (Marangoni, et al., 2007, J. Exp. Med. 204(2):369-380).

Overall, these studies identify a complex role for LF in immunomodulation which may represent the requirement for initial activation of the inflammatory response in an orchestrated manner, together with the requirement for a dampening of inflammation in an effort to prevent a sustained pathophysiological outcome.

In spite numerous publications regarding the evidence of LF efficacy in a multitude of inflammatory-based disease models, there is no discussion in the literature addressing the biological mechanism as to how LF induces this observed protection. Further, no experimental evidence has been reported regarding the underlying molecular events or the physiological effects of LF treatment. There remains an unmet need in the art for treatment of autoimmune diseases using the lactoferrin compositions and methods described herein.

According to the present disclosure, compositions comprising lactoferrin are useful for driving specific gene regulation in CD4⁺ T cells in a manner that their moves their phenotype away from Th17 towards a Treg fate. Specifically, compositions comprising lactoferrin are useful in upregulating genes (Fosl1, Foxp3, Ikzf2, Irf1, Irf4, Tgif) involved in generation of regulatory T cells and concomitantly downregulating canonical regulators of Th17 phenotype (Il-17a, Il17re, Rora), for treatment of autoimmune diseases and disorders.

The foregoing description of the related art and limitations related therein are intended to be illustrative and not exclusive. Other limitations of the related art will become apparent to those of skill in the art upon a reading of the instant specification and a study of the drawings.

### BRIEF SUMMARY

The present disclosure provides compositions comprising lactoferrin, including a full-length lactoferrin polypeptide from any mammal, or a peptide/polypeptide fragment thereof, either purified from the mammal, or mammalian secretions (e.g. milk, tears, saliva, etc.) or recombinantly expressed in a heterologous organism or species, and methods of using said compositions for the modulation of certain T cell subtypes and their activities.

The foregoing examples of the related art and limitations related therewith are intended to be illustrative and not exclusive. Other limitations of the related art will become apparent to those of skill in the art upon a reading of the specification and a study of the drawings.

In one aspect, a pharmaceutical composition comprising lactoferrin (LF) is provided for administration to a subject having an autoimmune disease or disorder, wherein said composition modulates specific T cell populations/phenotype/activity in a subject having a neurodegenerative or autoimmune disease, and improves the balance between anti-inflammatory (Th2) cytokine producing cells and pro-inflammatory (Th1) or Th17 cells, and skews naïve T cells toward a pro-regulatory phenotype, thereby treating, ameliorating or preventing the neurodegenerative or autoimmune disease or disorder.

In some embodiments, the lactoferrin is 40-50% iron saturated. In some embodiments, the lactoferrin is 25%-75% iron saturated. In some embodiments, the lactoferrin is apo-lactoferrin (apo-LF). In some embodiments, the lactoferrin is holo-lactoferrin (holo-LF).

In some embodiments, the lactoferrin composition upregulates genes responsible for generation of Treg cells and downregulates genes responsible for generation of IL-17 (which produces Th17 cells). In some embodiments, upon treatment with the presently disclosed lactoferrin composition according to the methods described herein, CD4⁺CD25⁻naïve T cells are skewed toward a pro-regulatory phenotype.

In some embodiments, the autoimmune disease or disorder is selected from inflammatory bowel disease (IBD), rheumatoid arthritis (RA), systemic lupus erythematosus, multiple sclerosis (MS), amyotrophic lateral sclerosis (ALS), anti-glomerular basement membrane nephritis (Goodpasters syndrome, GPS) Bullous pemphigoid, dermatitis herpetiformis (DH), epidermolysis bullosa acquisita, linear IgA dermatosis, pemphigus vulgaris, Addison's disease, polyglandular autoimmune syndrome (PGAS), autoimmune pancreatitis (AIP), type 1 diabetes (T1D), autoimmune thyroiditis, Ord's thyroiditis, Graves' disease, Sjögren's syndrome, autoimmune enteropathy, Coeliac disease, antiphospholipid antibody syndrome (APS/APLS, Highes syndrome), autoimmune haemolytic anaemia (AIHA, autoimmune lymphoproliferative syndrome, ALPS, Canale-Smith syndrome) autoimmune neutropenia, idiopathic thrombocytopenic purpura (ITP), Evan's syndrome, pernicious anemia, adult-onset Still's disease (AOSD), childhoon arthritis (juvenile arthritis, JA), psoriatic arthritis, rheumatic heart disease (RHD), Myasthenia gravis, acute coronary syndrome (ACS, including unstable angina (UA) and acute myocardial infarction (AMI)), chronic inflammatory demyelinating polyneuropathy, autoimmune uveitis, Graves' ophthalmopathy, Granulomatosis with polyangiitis (GPA), vasculitis, autoimmune hepatitis, autoimmune inner ear disease, Primary Biliary Cirrhosis, and neurodegenerative diseases including Parkinson's disease, Alzheimer's disease, cognitive decline in the elderly resulting from chronic inflammation, and HIV-associated inflammation and/or neurodegeneration.

In one aspect, a pharmaceutical composition comprising human lactoferrin (hLF) comprising the amino acid sequence set forth hereinbelow as SEQ ID NO: 2 is provided, wherein said hLF protein in the composition is greater than 85% by weight of the composition. In some embodiments, the LF in the composition is greater than 90% by weight of the composition. In some embodiments, the LF in the composition is greater than 91% by weight of the composition. In some embodiments, the LF in the composition is greater than 92% by weight of the composition. In some embodiments, the LF in the composition is greater than 93% by weight of the composition. In some embodiments, the LF in the composition is greater than 94% by weight of the composition. In some embodiments, the LF in the composition is greater than 95% by weight of the composition. In some embodiments, the LF in the composition is greater than 96% by weight of the composition. In some embodiments, the LF in the composition is greater than 97% by weight of the composition. In some embodiments, the LF in the composition is greater than 98% by weight of the composition. In some embodiments, the LF in the composition is greater than 99% by weight of the composition. In some embodiments, the LF is in a composition comprising an aqueous buffer. In some embodiments, the LF composition is formulated to be delivered as a tablet or pill. In some embodiments, the tablet or pill has a gelatin coating. In some embodiments, the LF composition is formulated for oral delivery to reach a specific section of GI tract. In some embodiments, the LF composition is formulated for injectable delivery.

In one aspect, a method of treating or ameliorating an autoimmune disorder by administering the pharmaceutical composition comprising lactoferrin (LF) to a subject in need of treatment is provided.

In some embodiments, a method of treating or ameliorating chronic inflammation (CI) in an adolescent or an adult is provided.

Antiretroviral therapy (ART) effectively and durably suppresses HIV replication, leads to immune recovery (increasing CD4+ T-cell counts), prolonged life expectancy, and has fundamentally changed the spectrum of morbidity and mortality among HIV positive persons. Among well-treated patients with levels of HIV RNA below the level of detection, non-AIDS-related conditions such as atherosclerotic cardiovascular disease (CVD), cancer, liver disease, end-stage renal disease, bone disease and subclinical neurocognitive dysfunction are now a more common cause of morbidity and mortality in current clinical practice than AIDS itself. Of these, CVD and cancer constitute the vast majority of clinical events.

Excess risk for serious non-AIDS-related conditions among HIV positive persons is due to multiple factors, including a higher burden of traditional risk factors, ART toxicity and chronic inflammation. Recent data from a number of epidemiologic studies have shown that key biomarkers of inflammation and coagulation and markers cellular activation—all of which improve with ART but do not normalize to uninfected population levels—predict risk for CVD, cancer, and mortality. Levels of two such markers, D-dimer and interleukin-6 (IL-6), were strongly associated with risk for CVD and all-cause mortality, and IL-6 levels also predict cancer risk.

Thus, the lactoferrin compositions of the present disclosure may be a useful means of lowering chonic inflammation in HIV-positive patients. HIV related inflammation has been associated with upregulation of D-dimer and IL-6 markers, and lactoferrin may be useful as a therapeutic in the modulation of Treg populations. Studies are underway to assess the ability of recombinant human lactoferrin to reduce immune activation and coagulation among HIV-positive patients, and to reduce risk for non-AIDS-defining conditions (e.g., CVD) among virally suppressed HIV positive participants, by determining the treatment effect on an IL-6/D-dimer score that itself is associated with risk for clinical events.

In one aspect, a method of skewing CD4⁺CD25⁻ naïve T cells toward a pro-regulatory phenotype, as measured by intracellular cytokine staining to observe an increase in IFNγ and IL-10⁺ cells by administering a pharmaceutical composition comprising lactoferrin (LF) is provided.

In one aspect, a method of inducing Treg phenotype and/or activity by administering the pharmaceutical composition comprising LF is provided.

In one aspect, a method of expanding Treg cell populations by administering the pharmaceutical composition comprising LF is provided.

In one aspect, a method of reducing Th1/Th17 T cell phenotype by administering the pharmaceutical composition comprising LF is provided.

In one aspect, a method to induce and/or maintain remission of an autoimmune disease or disorder by administering the pharmaceutical composition comprising LF is provided.

In one aspect, a method of upregulating genes (Fosl1, Foxp3, Ikzf2, Irf1, Irf4, Tgif) involved in Treg generation, and concomitantly downregulating genes (Il-17a, Il17re, Rora) involved in regulating Th17 phenotype is provided.

In some embodiments, the lactoferrin is native lactoferrin isolated and purified from a mammal or mammalian secretion. In some embodiments, the lactoferrin is human lactoferrin. In some embodiments, the lactoferrin is isolated and purified from a cow or from cow's milk. In some embodiments, the lactoferrin is bovine lactoferrin. In some embodiments, the lactoferrin is mammalian lactoferrin recombinantly expressed in E. Coli. In some embodiments, the lactoferrin is recombinant human lactoferrin (rhLF) expressed in CHO cells. In some embodiments, the lactoferrin is recombinant human lactoferrin (rhLF) expressed in HEK cells. In some embodiments, the lactoferrin is recombinant human lactoferrin (rhLF) expressed by yeast cells. In some embodiments, the lactoferrin is a monocot-derived recombinant human lactoferrin (rhLF) polypeptide. In some embodiments, the LF composition comprises 0.1% or more monocot plant-derived components). In some embodiments, the recombinant lactoferrin is expressed in a mature, transgenic monocot seed that yields, upon extraction of the ground seed using an aqueous medium, a total soluble protein fraction containing at least 3% by total protein weight of lactoferrin protein or polypeptide fragment thereof. In some embodiments, the monocot seed comprises a total soluble protein fraction of at least 4% lactoferrin by total protein weight. In some embodiments, the monocot seed comprises a total soluble protein fraction of at least 5% lactoferrin by total protein weight. In some embodiments, the monocot seed comprises a total soluble protein fraction of at least 6%, at least 7%, at least 8%, at least 9%, or at least 10% lactoferrin by total protein weight. In some embodiments, the monocot seed comprises a total soluble protein fraction of at least 15% lactoferrin by total protein weight. In some embodiments, the monocot seed comprises a total soluble protein fraction of at least 20% lactoferrin by total protein weight.

Additional embodiments of the presently disclosed methods and compositions, and the like, will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present invention. Additional aspects and advantages of the present invention are set forth in the following description and claims, particularly when considered in conjunction with the accompanying examples and drawings.

### BRIEF DESCRIPTION OF THE DRAWINGS

FIG. 1 shows terminal sections of the ilea treated with anti-TNF (“aTNF”); 50 mg/kg LF (“LF50”); 500 mg/kg LF (LF500). (A) presents scoring on parameters of active and chronic inflammation, and villous architecture; (B) presents tissues from each of the treatment groups.

FIG. 2 shows the results of ELISA assays demonstrating that LF modulates cytokine expression in ileal explants, rhLF Reduces Cytokine Secretion in Ileal Explants from TNF^{ΔARE} Mice.

FIG. 3 show that LF treatment of TNF^{ΔARE} mice results in decreased CD4+ burden in A) the lamina propria and B) mesenteric lymph nodes, while T cells showed C) an increase in IL-10 producing cells and D) a decrease in IL-17 producing cells at the lamina propria.

FIG. 4 presents evidence that LF skews CD4+CD25Neg naïve T cells toward a pro-regulatory phenotype (Treg), Conversion of CD4⁺CD25^{Neg} T cells under polarizing conditions is skewed by LF to a pro-regulatory phenotype.

FIG. 5 illustrates that LF differentially regulates pro- and anti-inflammatory gene expression in isolated CD4+ murine cells in vivo. In FIG. 5A, CD4+ T cells were negatively selected from spleens of healthy C57/BL6 mice using magnetic sorting. Proliferating T cells were stimulated with 1 μM LF for 24 hrs, and RNA harvested and cDNA prepared. Qualitative PCR (qPCR) was carried out a heatmap generated to show upregulated (light-dark green) and downregulated genes (yellow-red).

FIG. 6 show that LF drives the expression and secretion of IL-2 in activated primary murine T cells. A) shows primary T cells isolated from spleens of healthy C57/BL6 mice and enriched for CD4+ cells by negative selection, in which proliferation was induced by plate-bound antiCD3/CD28 in the presence or absence of 1 uM rhLF over 2, 4, 6, 18 and 24 h time periods. LF enhances IL-2 gene expression as early as 6h, which is sustained through at least 24h post-treatment. B) and C) show that LF significantly augments IL-2 secretion in activated (6B) and non-activated (6C) human Jurkat T cells in a dose-dependent manner. D) demonstrates that LF initiates the secretion of IL-2 in non-activated primary peripheral human T cells.

FIG. 7 illustrates that rhLF induces secretion of IL-10 and compares the effect to LF isolated from bovine colostrum (Bovine LF) or from human milk (Human LF) in human CD3⁺ lymphocytes over a 72 hour treatment period.

FIG. 8 shows that rhLF induces phosphorylation of the p38 and Erk signaling cassettes of the MAPK cascade. CD3⁺ cells were treated with either 50 uM or 100 uM various forms of LF over a 72h period and Western blotting analysis carried out for phopshoylated Erk (pErk) or phosphorylated p38 (p-p38).

FIG. 9A shows that rhLF acts through the Erk signaling cascade to induce secretion of IL-10 in freshly isolated human CD3+ cells.

FIG. 9B shows that rhLF acts through the Erk signaling cascade to induce secretion of IL-2 in the Jurkat cell line.

FIG. 10 illustrates that subcutaneous (S.Q.) and oral administration of rhLF results in Treg homing to intestinal tissues and associated lymphoid organs.

FIG. 11 shows the results of a blinded scoring of overall inflammation in TNF^{ΔARE} mice treated continuously or intermittently with rhLF; continual oral gavage of rhLF reverses inflammation in TNF^{ΔARE} mice demonstrated in the 55 mg/kg group. *, p<0.05; ***, p<0.001, Student's t-test.

FIG. 12A shows rhLF treatment of DSS mice significantly decreases FITC-dextran mw 4,000 Da transport across the intestinal epithelial barrier to the serum. Reduced inflammatory indices and histopathological damage are observed in DSS mice treated with rhLF.

FIG. 12B shows weight loss measurements demonstrates mice are protected following administration of 500 mg/kg/day rhLF (*p<0.05 two-way ANOVA, with Bonferonni's post-test.

FIG. 12C shows administration of high dose LF results in protection from mucosal injury (preservation of colon length) (*p<0.05, student's t-test).

FIG. 12D shows H&E staining of the colon reveals a loss of epithelial integrity, loss of crypt architecture, absence of goblet cells, and immune cell infiltration upon insult with DSS alone, which is rescued by rhLF administration at 100, 250 and 500 mg/kg/day).

FIG. 13 shows that reduced overall T cell burden and promotion of Treg phenotype are observed in DSS mice treated with rhLF; CD4⁺ cell infiltration is reduced in the rhLF treated animals vs. PBS control in (A) MLN but not (B) LP. CD4⁺ T lymphocytes from the mesenteric (C) MLN and (D) LP of DSS-treated mice were isolated using negative selection and stimulated in the presence of PMA, Ionomoycin, Brefeldin A for 4h and analyzed by flow cytometry. An increase in Tregs (CD4⁺ FOXP3⁺) was induced in mice treated with rhLF to a small degree in the and more dramatically in colonic LP. *, p<0.05, student's t-test; **, p<0.01, student's t-test

FIG. 14 shows a shift in the balance of CD4⁺ T cells in a DSS mouse model of colitis; rhLF reduced expression of the pro-inflammatory cytokine IL-17 in A) MLN CD4⁺ cells and B) LP CD4⁺ cells with concomitant reduction in the pro-inflammatory cytokine IFNγ in C) LP CD4⁺ cells. Conversely, IL-10 producing CD4⁺ were demonstrated to be increased in D) MLN of mice administered rhLF. However, in CD4⁺ cells isolated from E) colonic LP, no effect on IL-10 production was demonstrated at low dose administration of rhLF, and a decrease demonstrated at the highest dose of rhLF. *, p<0.05; **, p<0.01.

### BRIEF DESCRIPTION OF THE SEQUENCES

The nucleic acid coding sequence encoding human lactoferrin is represented herein as SEQ ID NO: 1.

The amino acid sequence of a human lactoferrin protein, having a glutamine at position 14 instead of an asparagine is represented herein as SEQ ID NO: 2.

SEQ ID NO. 3 identifies a nucleic acid sequence encoding human lactoferrin that is codon-optimized for expression in rice.

SEQ ID NO. 4 identifies the amino acid sequence of human lactoferrin protein encoded by the codon-optimized SEQ ID NO. 3 above.

GenBank Accession AH000852.2 presents the nucleic acid coding sequence encoding bovine lactoferrin, identified herein as SEQ ID NO: 5.

The amino acid sequence of a bovine lactoferrin protein (Protein ID AAA21722.1) is represented herein as SEQ ID NO: 6.

The nucleotide sequence of Accession No. AY360320.1, encoding a neutrophil lactoferrin (SEQ ID NO: 7).

SEQ ID NO: 8 identifies the amino acid sequence of a peptide from human neutrophil lactoferrin, encoded by a transcript having a novel region of splice-variance.

SEQ ID NO: 9 identifies the amino acid sequence of human neutrophil lactoferrin protein, Accession No. AAR12276.1.

### DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

### I. DEFINITIONS

As used in this specification, the singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

Where a range of values is provided, it is intended that each intervening value between the upper and lower limit of that range and any other stated or intervening value in that stated range is encompassed within the disclosure and specifically disclosed. For example, if a range of 1 μM to 8 μM is stated, it is intended that 2 μM, 3 μM, 4 μM, 5 μM, 6 μM, and 7 μM are also explicitly disclosed, as well as the range of values greater than or equal to 1 μM and the range of values less than or equal to 8 μM.

A variety of host expression vector systems may be utilized to express peptides described herein. These include, but are not limited to, microorganisms such as bacteria transformed with recombinant bacteriophage DNA or plasmid DNA expression vectors containing an appropriate coding sequence; yeast or filamentous fungi transformed with recombinant yeast or fungi expression vectors containing an appropriate coding sequence; insect cell systems infected with recombinant virus expression vectors (e.g., baculovirus) containing an appropriate coding sequence; plant cell systems infected with recombinant virus expression vectors (e.g., cauliflower mosaic virus or tobacco mosaic virus) or transformed with recombinant plasmid expression vectors (e.g., Ti plasmid) containing an appropriate coding sequence; or animal cell systems.

“Recombinant,” when used with reference to, e.g., a cell, nucleic acid, polypeptide, expression cassette or vector, refers to a material, or a material corresponding to the natural or native form of the material, that has been modified by the introduction of a new moiety or alteration of an existing moiety, or is identical thereto but produced or derived from synthetic materials. For example, recombinant cells express genes that are not found within the native (non-recombinant) form of the cell (i.e., “exogenous nucleic acids”) or express native genes that are otherwise expressed at a different level, typically, under-expressed or not expressed at all.

Recombinant techniques can include, e.g., use of a recombinant nucleic acid such as a cDNA encoding a protein or an antisense sequence, for insertion into an expression system, such as an expression vector; the resultant construct is introduced into a cell, and the cell expresses the nucleic acid, and the protein, if appropriate. Recombinant techniques also encompass the ligation of nucleic acids to coding or promoter sequences from different sources into one expression cassette or vector for expression of a fusion protein, constitutive expression of a protein, or inducible expression of a protein.

“Exogenous” as in “exogenous nucleic acid” refers to a molecule (e.g., nucleic acid or polypeptide) that has been isolated, synthesized, and/or cloned, in a manner that is not found in nature, and/or introduced into and/or expressed in a cell or cellular environment other than or at levels or forms different than the cell or cellular environment in which said nucleic acid or protein can be found in nature. The term encompasses both nucleic acids originally obtained from a different organism or cell type than the cell type in which it is expressed, and also nucleic acids that are obtained from the same organism, cell, or cell line as the cell or organism in which it is expressed.

“Heterologous” when used with reference to a nucleic acid or polypeptide, indicates that a sequence that comprises two or more subsequences which are not found in the same relationship to each other as normally found in nature, or is recombinantly engineered so that its level of expression, or physical relationship to other nucleic acids or other molecules in a cell, or structure, is not normally found in nature. For instance, a heterologous nucleic acid is typically recombinantly produced, having two or more sequences from unrelated genes arranged in a manner not found in nature; e.g., a nucleic acid open reading frame (ORF) can be operatively linked to a promoter sequence inserted into an expression cassette, e.g., a vector. As another example, a polypeptide can be linked to tag, e.g., a detection- and purification-facilitating domain, as a fusion protein.

“Coding sequence” refers to that portion of a nucleic acid (e.g., a gene) that encodes an amino acid sequence of a protein.

“Gene” refers to a nucleic acid fragment that expresses a specific protein, including regulatory sequences preceding (5′ non-coding sequences) and following (3′ non-coding sequences) the coding sequence.

“Native gene” refers to a gene as found in nature with its own regulatory sequences.

“Chimeric gene” refers any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature.

“Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes.

“Transgene” is a gene that has been introduced into the genome by a transformation procedure.

“Operably linked” refers to a functional relationship between two or more nucleic acid (e.g., DNA) segments. Typically, it refers to the functional relationship of a transcriptional regulatory sequence to a transcribed sequence. For example, a promoter is operably linked to a coding sequence, such as a nucleic acid, if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

“Control sequence” refers to polynucleotide sequences which are necessary to effect the expression of coding and non-coding sequences to which they are ligated. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, components whose presence can influence expression, and can also include additional components whose presence is advantageous, for example, leader sequences and fusion partner sequences.

“Primer” and “probe” refer to a nucleic acid molecule including DNA, RNA and analogs thereof, including protein nucleic acids (PNA), and mixtures thereof. Such molecules are typically of a length such that they are statistically unique (i.e., occur only once) in the genome of interest. Generally, for a probe or primer to be unique in the human genome, it contains at least 14, 16 or contiguous nucleotides of a sequence complementary to or identical to a gene of interest. Probes and primers can be 10, 20, 30, 50, 100 or more nucleic acids long.

“Recombinant host cell” refers to a cell that comprises a recombinant nucleic acid molecule. Thus, for example, recombinant host cells can express genes that are not found within the native (non-recombinant) form of the cell.

“Modulation” refers to the capacity to either enhance or inhibit a functional property of biological activity or process (e.g., increasing or decreasing T cell subtypes; enhancing or inhibiting autoimmunity; enhancing or inhibiting pro-inflammatory activities, etc.). Such enhancement or inhibition may be contingent on the occurrence of a specific event, such as activation of a signal transduction pathway, and/or may be manifest only in particular cell types.

“Physiological conditions” or “physiological solution” refers to an aqueous environment having an ionic strength, pH, and temperature substantially similar to conditions in an intact mammalian cell or in a tissue space or organ of a living mammal. Typically, physiological conditions comprise an aqueous solution having about 150 mM NaCl, pH 6.5-7.6, and a temperature of approximately 22-37 degrees C. Generally, physiological conditions are suitable binding conditions for intermolecular association of biological macromolecules. For example, physiological conditions of 150 mM NaCl, pH 7.4, at 37 degrees C. are generally suitable.

“Associated” refers to coincidence with the development or manifestation of a disease, condition or phenotype. Association may be due to, but is not limited to, genes responsible for housekeeping functions whose alteration can provide the foundation for a variety of diseases and conditions, those that are part of a pathway that is involved in a specific disease, condition or phenotype and those that indirectly contribute to the manifestation of a disease, condition or phenotype.

“Mature protein” refers to a post-translationally processed polypeptide; i.e., one from which any pre- or propeptides present in the primary translation product has been removed. “Precursor” protein refers to the primary product of translation of mRNA; i.e., with pre- and propeptides still present. Pre- and propeptides may be but are not limited to intracellular localization signals.

“3′ non-coding sequences” refer to nucleotide sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al. ((1989) Plant Cell 1:671-680).

“Translation leader sequence” refers to a nucleotide sequence located between the promoter sequence of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences have been described (Turner and Foster (1995) Molecular Biotechnology 3:225).

“Position corresponding to” refers to a position of interest (i.e., base number or residue number) in a nucleic acid molecule or protein relative to the position in another reference nucleic acid molecule or protein. Corresponding positions can be determined by comparing and aligning sequences to maximize the number of matching nucleotides or residues, for example, such that identity between the sequences is greater than 90%, greater than 95%, greater than 96%, greater than 97%, greater than 98% or greater than 99%. The position of interest is then given the number assigned in the reference nucleic acid molecule. For example, if a particular polymorphism in Gene-X occurs at nucleotide 2073 of SEQ ID No. X, to identify the corresponding nucleotide in another allele or isolate, the sequences are aligned and then the position that lines up with 2073 is identified. Since various alleles may be of different length, the position designate 2073 may not be nucleotide 2073, but instead is at a position that “corresponds” to the position in the reference sequence.

“Transgenic” refers to any organism, prokaryotic or eukaryotic, which contains at least a cell bearing a heterologous or recombinant nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The recombinant nucleic acid molecule may be integrated within a chromosome, or it may be extrachromosomally replicating DNA.

“Heterologous nucleic acid” refers to nucleic acid which has been introduced into host cells from another source, or which is from a plant source, including the same plant source, but which is under the control of a promoter that does not normally regulate expression of the heterologous nucleic acid. “Heterologous peptide, polypeptide or protein” is a peptide, polypeptide or protein encoded by a heterologous nucleic acid, or a peptide/polypeptide/protein from one class, order, family, genus, species or organism introduced into a different class, order, family, genus, species or organism. The peptides, polypeptides or proteins described herein include lactoferrin proteins of mammalian origin, including human and bovine lactoferrins, either purified from the species of origin or heterologously expressed using recombinant DNA technology. Animals such as sheep, cattle, goats, pigs, horses and domestic animals, including cats and dogs, are contemplated to be useful as host organisms for the production of lactoferrin or a part thereof.

“Percentage of sequence identity” and “percentage homology” are used interchangeably herein to refer to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)). Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 3389-3402, respectively. Software for performing BLAST analyses is publicly available through the National Center for Biotechnology Information website. This algorithm involves first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (Altschul et al, supra). These initial neighborhood word hits act as “seeds” for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

While all of the above mentioned algorithms and programs are suitable for a determination of sequence alignment and % sequence identity, for purposes of the disclosure herein, determination of % sequence identity will typically be performed using the BESTFIT or GAP programs in the GCG Wisconsin Software package (Accelrys, Madison Wis.), using default parameters provided.

General and specific techniques for producing proteins from plant cells may be obtained from the following applications, each of which is incorporated herein in its entirety by reference: U.S. patent application Ser. No. 09/847,232 (“Plant Transcription Factors and Enhanced Gene Expression”); U.S. patent application Ser. No. 10/077,381 (“Expression of Human Milk Proteins in Transgenic Plants”); U.S. patent application Ser. No. 10/411,395 (“Human Blood Proteins Expressed in Monocot Seeds”); U.S. patent application Ser. No. 10/639,779 (“Production of Human Growth Factors in Monocot Seeds”); U.S. patent application Ser. No. 10/639,781 (“Method of Making an Anti-infective Composition for Treating Oral Infections”); and international application no. PCT/US2004/041083 (“High-level Expression of Fusion Polypeptides in Plant Seeds Utilizing Seed-Storage Proteins as Fusion Carriers”).

The term “plant” includes reference to whole plants, plant organs (for example, leaves, stems, roots, etc.), seeds, and plant cells and progeny of same. The class of plants that can be used in the methods of the present invention is generally as broad as the class of higher plants amenable to transformation techniques, including both monocotyledonous and dicotyledonous plants. The plant can be a monocot plant. The plant is often a cereal, selected from the group consisting of rice, barley, wheat, oat, rye, corn, millet, triticale and sorghum. The term “mature plant” refers to a fully differentiated plant.

As used herein, “plant cell” refers to any cell derived from a plant, including undifferentiated tissue (e.g., callus) as well as plant seeds, pollen, propagules, embryos, suspension cultures, meristematic regions, leaves, roots, shoots, gametophytes, sporophytes and microspores. Plant cells include, without limitation, cells in seeds, suspension cultures, embryos, meristematic regions, callus tissue, leaves roots shoots, gametophytes, sporophytes, pollen, and microspores.

The term “seed” is meant to encompass all seed components, including, for example, the coleoptile and leaves, radicle and coleorhiza, scutulum, starchy endosperm, aleurone layer, pericarp and/or testa, either during seed maturation and seed germination. In the context of the present disclosure, the terms “seed” and “grain” may be used interchangeably.

The term “seed in a form for use as a food or food supplement” includes, but is not limited to, seed fractions such as de-hulled whole seed, flour (seed that has been de-hulled by milling and ground into a powder) a seed protein extract (where the protein fraction of the flour has been separated from the carbohydrate fraction) and/or a purified protein fraction derived from the transgenic grain.

“Seed components” refers to carbohydrate, protein, and lipid components extractable from seeds, typically mature seeds.

The term “seed product” includes, but is not limited to, seed fractions such as de-hulled whole seed, a flour (seed that has been de-hulled by milling and ground into a powder), a seed extract, a protein extract (where the protein fraction of the flour has been separated from the carbohydrate fraction), a malt (including malt extract or malt syrup) and/or a purified protein fraction derived from the transgenic grain.

The conditions used in concentration and diafiltration will vary depending on volume, speed, cost, etc., and are routine in the art. For example lactoferrin-expressing transgenic rice flour can be mixed with extraction buffer at about 100 g/L for about 1 hour using a magnetic stir bar. In a two-liter beaker, PBS, pH7.4 plus 0.35 M NaCl can be used as the extraction buffer. Alternatively, the extraction buffer may be 0.5 M ammonium bicarbonate. Other extraction buffers can also be used to extract recombinant proteins expressed in transgenic rice grains, for example Tris buffer, ammonium acetate, depending on applications. For example, in preparing recombinant human LF from monocot seeds, iron may be added to the extraction buffer and the buffer is set at a pH so that the apo-LF (lacking iron) can pick up iron during the extraction process. Under these conditions, LF can become saturated with iron (holo-LF). In some embodiments, a buffer lacking of iron and having a pH resulting in iron release from LF is used to produce apo-LF.

Various salts, buffers, etc. may be present in the lactoferrin composition. In some embodiments, transferrin (having a different sequence and structure from lactoferrin) is used as a negative control. Other “modulators” of T cell activity or agents (e.g., 5-ASA drugs, antibiotics, corticosteroids, disease-modifying antirheumatic drugs (DMARDs) such as hydroxychloroquine, small molecule immune modulators and biologics, such as SR1001, SR2211 and diphenylpropanamide compounds that act as selective RORyt inhibitors) which can also induce or maintain remission of autoimmune diseases or disorders may be included in the composition; these other agents may not have a primary mode of action in Treg modulation, but some biologics and immune suppressors have been reported to affect levels of Treg, possibly via secondary actions. Tregs are strongly induced by experimental therapies such as retinoic acid, and histone deacetylase inhibitors but these therapies are not yet approved or standard of care.

A 28-day repeated dose study of oral toxicity of recombinant human holo-lactoferrin (holo-rhLF) in Wistar rats was conducted using holo-rhLF expressed in rice grain, extracted, purified and saturated with iron. Upon oral administration (via gavage) to rats at 1000, 500 and 100 mg/kgbw/day, the holo-rhLF was well-tolerated and did not appear to be toxic. A significantly greater total iron binding capacity (TIBC) was detected in the blood of male animals dosed with holo-rhLF. Serum was analyzed for the presence of IgG and IgE antibodies; demonstrating low levels of IgG antibodies to the human protein, but no increase in IgE antibodies. There was no increase in serum lactoferrin levels. There were no treatment related, toxicologically relevant changes in clinical signs, growth, food consumption, hematology, clinical chemistry, organ weights or pathology. The no observed adverse effect level (NOAEL) is greater than 1000 mg/kg/day (Cerven, et al., 2008, Regul. Toxicol. Pharmacol. 52:174-9).

“Plant-derived” refers to a recombinant expression product (nucleic acid or polypeptide) that is not endogenous to the plant, but is expressed in the transgenic plant upon introduction of a recombinant nucleic acid sequence.

“Plant-derived food ingredients” refers to plant-derived food stuff, typically monocot grain, but also including, separately, lectins, gums, sugars, plant-produced proteins and lipids, that may be blended or combined, alone or in combination with one or more plant-derived ingredients, to form an edible food.

The term “nutritionally enhanced food” refers to a food composition, typically a processed food, to which a seed-produced lactoferrin protein composition, or partially- or significantly-purified lactoferrin protein, has been added, in an amount effective to confer some health benefit, such as improved gut health, reversal or decreased symptoms of an autoimmune disease or disorder, or iron transport, to a human consuming the food.

“Seed maturation” or “grain development” refers to the period starting with fertilization in which metabolizable reserves, e.g., sugars, oligosaccharides, starch, phenolics, amino acids, and proteins, are deposited, with and without vacuole targeting, to various tissues in the seed (grain), e.g., endosperm, testa, aleurone layer, and scutellar epithelium, leading to grain enlargement, grain filling, and ending with grain desiccation.

“Substantially unpurified form,” as applied to a seed extract composition comprising lactoferrin protein or polypeptide means that the protein or proteins present in the extract are present in an amount less than 50% by weight, typically between 0.1 and 10 percent by weight.

In some embodiments, the seed storage protein can be from a monocot plant. In some embodiments, the seed storage protein is selected from the group consisting of rice globulins, rice glutelins, oryzins, prolamines, barley hordeins, wheat gliadins and glutenins, maize zeins and glutelins, oat glutelins, sorghum kafirins, millet pennisetins, or rye secalins. For example, rice globulin and rice glutelin are suitable. The seed storage protein may be at the N-terminal or C-terminal side of the lactoferrin protein in the fusion protein. In some embodiments, the seed storage protein is located at the N-terminal side of the lactoferrin protein.

Plant cells or tissues are transformed with expression constructs using a variety of standard techniques. In some embodiments, the vector sequences are stably integrated into the host genome. Suitable plants are those that have been transformed with a lactoferrin expression vector, or have been grown from a plant cell that has been transformed with a lactoferrin expression vector, in accordance with the methods described herein, and express a lactoferrin fusion protein as a result of the transformation. Also suitable are plants that have been transformed with a lactoferrin expression vector, or have been grown from a plant cell that has been transformed with a lactoferrin expression vector, that are fertile and phenotypically normal and express a lactoferrin fusion protein.

As used herein, the terms “transformed” or “transgenic” with reference to a host cell means the host cell contains a non-native or heterologous or introduced nucleic acid sequence that is absent from the native host cell. Further, “stably transformed” in the context of the present disclosure means that the introduced nucleic acid sequence is maintained through two or more generations of the host, which may be due to integration of the introduced sequence into the host genome.

According to another aspect of the disclosure, plants that have been transformed with the lactoferrin expression vector exhibit growth that is comparable to a wild-type plant of the same species, or exhibit fertility that is comparable to a wild-type plant of the same species, or both. A transformed plant that exhibits comparable growth to a wild-type plant may produce at least 80% of the amount of total biomass produced by a wild-type plant grown under similar conditions, such as location (e.g., greenhouse, field, etc.), soil type, nutrients, water, and exposure to sunlight. The transformed plant may produce at least 85%, or at least 90%, or at least 95% of the amount of total biomass produced by a wild-type plant grown under similar conditions. A transformed plant that exhibits comparable fertility to a wild-type plant may produce at least 80% of the amount of offspring produced by a wild-type plant grown under similar conditions, such as location (e.g., greenhouse, field, etc.), soil type, nutrients, water, and exposure to sunlight. In some embodiments, the transformed plant produces at least 85%, at least 90%, or at least 95% of the amount of offspring produced by a wild-type plant grown under similar conditions.

According to a further aspect of the disclosure, the plants transformed with the lactoferrin gene construct are comparable to a wild-type plant of the same species and express the lactoferrin protein as a result of the transformation. In some embodiments, the transformed plants express the lactoferrin fusion protein at high levels, e.g., 2%, 3%, 5%, 8%, 9%, 10%, or 20% or greater of the total soluble protein in the seeds of the plant.

The method used for transformation of host plant cells is not critical to the present disclosure. For commercialization of the heterologous peptide or polypeptide expressed in accordance with the present disclosure, the transformation of the plant can be permanent, i.e., by integration of the introduced expression constructs into the host plant genome, so that the introduced constructs are passed onto successive plant generations. The skilled artisan will recognize that a wide variety of transformation techniques exist in the art, and new techniques are continually becoming available.

Any technique that is suitable for the target host plant may be employed within the scope of the present disclosure. For example, the constructs can be introduced in a variety of forms including, but not limited to, as a strand of DNA, in a plasmid, or in an artificial chromosome. The introduction of the constructs into the target plant cells can be accomplished by a variety of techniques, including, but not limited to calcium-phosphate-DNA co-precipitation, electroporation, microinjection, Agrobacterium-mediated transformation, liposome-mediated transformation, protoplast fusion or microprojectile bombardment. The skilled artisan can refer to the literature for details and select suitable techniques for use in the methods of the present disclosure.

Transformed plant cells are screened for the ability to be cultured in selective media having a threshold concentration of a selective agent. Plant cells that grow on or in the selective media are typically transferred to a fresh supply of the same media and cultured again. The explants are then cultured under regeneration conditions to produce regenerated plant shoots. After shoots form, the shoots can be transferred to a selective rooting medium to provide a complete plantlet. The plantlet may then be grown to provide seed, cuttings, or the like for propagating the transformed plants. Suitable selectable markers for selection in plant cells include, but are not limited to, antibiotic resistance genes, such as kanamycin (nptll), G418, bleomycin, hygromycin, chloramphenicol, ampicillin, tetracycline, and the like. Additional selectable markers include a bar gene which codes for bialaphos resistance; a mutant EPSP synthase gene which encodes glyphosate resistance; a nitrilase gene which confers resistance to bromoxynil; a mutant acetolactate synthase gene (ALS) which confers imidazolinone or sulphonylurea resistance. The particular marker gene employed is one which allows for selection of transformed cells as compared to cells lacking the nucleic acid which has been introduced. In some embodiments, the selectable marker gene is one that facilitates selection at the tissue culture stage, e.g., an nptll, hygromycin or ampicillin resistance gene. Thus, the particular marker employed is not essential in the present compositions and methods.

The fusion protein may also be engineered to comprise at least one selective purification tag and/or at least one specific protease cleavage site for eventual release of the lactoferrin protein from the seed storage protein fusion partner, fused in translation frame between the lactoferrin protein and the seed storage protein. In some embodiments, the specific protease cleavage site may comprise enterokinase (ek), Factor Xa, thrombin, V8 protease, Genenase™, α-lytic protease or tobacco etch virus (TEV) protease. The fusion protein may also be cleaved chemically.

The expression of the heterologous peptide or polypeptide may be confirmed using standard analytical techniques such as Western blot, ELISA, PCR, HPLC, NMR, or mass spectroscopy, together with assays for a biological activity specific to the particular protein being expressed.

By “host cell for expression” is meant a cell containing a vector and supporting the replication and/or transcription and/or expression of a vector-encoded nucleic acid sequence. According to the present disclosure, the host cell is a plant cell. Other host cells may be used as secondary hosts, including bacterial, yeast, insect, amphibian or mammalian cells, to move DNA to a desired plant host cell.

Because the recombinant lactoferrin protein(s) of the present disclosure may be produced in plants, they may include plant glycosyl groups at one or more of the available N-glycosylation sites of the lactoferrin protein(s). For example, in one embodiment of the disclosure, a glycosylated lactoferrin protein(s) is produced in monocot seeds, such as rice, barley, wheat, oat, rye, corn, millet, triticale and sorghum. The lactoferrin protein may be glycosylated at all wild type or naturally occurring N-glycosylation sites, or at any subset of these sites, including at a single glycosylation site. Additional, new glycosylation sites may also be engineered into the expressed protein using recombinant DNA techniques. If a variant of a lactoferrin protein having a different number of N-glycosylation sites is utilized, it may be glycosylated at all or less than all of the N-glycosylation sites. Optionally, any or all plant glycosyl groups may be removed.

“Maturation-specific protein promoter” refers to a promoter exhibiting substantially up-regulated activity (greater than 25%) during seed maturation. The promoter may be from a maturation-specific monocot plant storage protein or an aleurone- or embryo-specific monocot plant gene. Other promoters may be used, however, and the choice of a suitable promoter is within the skill of those in the art. As such, the promoter can be a member selected from the group consisting of rice globulins, glutelins, oryzins and prolamines, barley hordeins, wheat gliadins and glutenins, maize zeins and glutelins, oat glutelins, sorghum kafirins, millet pennisetins, rye secalins, lipid transfer protein Ltp1, chitinase Chi26 and Em protein Emp1. In some embodiments, the promoter is selected from the group consisting of rice globulin Glb promoter and rice glutelin Gt1 promoter.

The seed-specific signal sequence used to replace the signal peptide from lactoferrin may be from a monocot plant, although other signal sequences may be utilized. In some embodiments, the monocot plant seed-specific signal sequence is associated with a gene selected from the group consisting of glutelins, prolamines, hordeins, gliadins, glutenins, zeins, albumin, globulin, ADP glucose pyrophosphorylase, starch synthase, branching enzyme, Em, and lea. In some embodiments, the monocot plant seed-specific signal sequence is a rice glutelin Gt1 signal sequence. Other monocot plant seed-specific signal sequence are associated with genes selected from the group consisting of α-amylase, protease, carboxypeptidase, endoprotease, ribonuclease, DNase/RNase, (1-3)-β-glucanase, (1-3)(1-4)-β-glucanase, esterase, acid phosphatase, pentosamine, endoxylanase, β-xylopyranosidase, arabinofuranosidase, β-glucosidase, (1-6)-β-glucanase, perioxidase, and lysophospholipase.

The promoter and signal sequence may be selected from those discussed supra. The type of promoter and signal sequence is not critical to this disclosure. In some embodiments, the signal sequence targets the attached fusion protein to a location such as an intracellular compartment, such as an intracellular vacuole or other protein storage body, mitochondria, or endoplasmic reticulum, or extracellular space, following secretion from the host cell.

The term “biological activity” refers to any biological activity typically attributed to a nucleic acid or protein by those skilled in the art. Examples of biological activities are enzymatic activity, ability to dimerize, fold or bind another protein or nucleic acid molecule, etc.

The nucleic acids of the present disclosure may be in the form of RNA or in the form of DNA, and include messenger RNA, synthetic RNA and DNA, cDNA, and genomic DNA. The DNA may be double-stranded or single-stranded, and if single-stranded may be the coding strand or the non-coding (anti-sense, complementary) strand.

As used herein, a “variant” is a nucleic acid, protein or peptide which is not identical to, but has significant homology (for example, 80%, 85%, 90%, 91%, 92%, 93%, 94%, 95%, 96%, 97%, 98% or 99% sequence identity) over the entire length of the wild type nucleic acid or amino acid sequence, as exemplified by sequences in the public sequence databases, such as GenBank. As used herein, a “protein, polypeptide or peptide fragment thereof” means the full-length protein or a portion of it having a wild type amino acid sequence usually at least 6, 7, 8, 9, 10, 11, 12, 13, 14 or 15 amino acids in length.

Peptic digestion of lactoferrin yields a cationic antimicrobial peptide (lactoferricin). (See Vogel, et al., 2002, Biochem. Cell Biol. 80(1):49-63; Mehra, et al., 2012, Exp. Dermatol. 21(10):778-782). Lactoferricin and another peptide, lactoferrampin, are derived from the whey protein lactoferrin, retain activity as antimicrobial agents. The location and solution structures of these two peptides, as well as the biological activities encompassing antiviral, antibacterial, antifungal and anti-inflammatory activities, have been studied. Various modifications of lactoferricin and lactoferrampin have been attempted, such as introducing big hydrophobic side-chains, employing special amino acids for synthesis, N-acetylization, amidation, and cyclization and constructing peptide chimera. A lactoferricin-lactoferrampin chimera has been reported (Yin, et al., 2014, Curr. Mol. Med. 14(9):1139-54). In some embodiments, lactoferricin is the LF fragment in the composition. In some embodiments, lactoferrampin is the LF fragment in the composition.

As used herein, a “mutant” is a mutated protein designed or engineered to alter properties or functions relating to glycosylation, protein stabilization and/or ligand binding.

As used herein, the terms “native” or “wild-type” relative to a given cell, polypeptide, nucleic acid, trait or phenotype, refers to the form in which that is typically found in nature.

The compositions and methods disclosed herein may employ lactoferrin protein recombinantly produced in a host plant seed. In some embodiments, the host plant is a monocot. In some embodiments, the host plant is a cereal, selected from the group consisting of rice, barley, wheat, oat, rye, corn, millet, triticale and sorghum. As used herein, “high levels of protein expression” means that the plant-expressed lactoferrin protein comprises about 2% or greater of the total soluble protein in the seed. Thus, for example, the yield of total soluble protein which comprises the lactoferrin protein targeted for production can be about 3% or greater, about 5% or greater, about 8% or greater, about 9% or greater, about 10% or greater, or about 20% or greater, of the total soluble protein found in the recombinantly engineered plant seed. Alternatively, the phrase “high yield expression” can mean that the level of expression of the recombinant lactoferrin protein in transgenic plant cells, plants or mature seeds is sufficiently high that a flour, extract or malt can be prepared from the seed directly without the need to purify the expressed protein.

In some embodiments, the lactoferrin protein constitutes at least 0.01 weight percent in the harvested seeds. In some embodiments, the lactoferrin protein constitutes at least 0.05 weight percent, and in some embodiments, at least 0.1 weight percent in the harvested seeds. Generally, “total soluble proteins” refers to the total amount of protein in a solution used to extract protein from a tissue. The phrase “total storage proteins” can encompass extractable and non-extractable protein. An average rice grain seed weight is 20-30 mg.

Suitable expression vectors for the production of lactoferrin or variant or fragment thereof are vectors which are capable of replicating in a host organism upon transformation. The vector may either be one which is capable of autonomous replication, such as a plasmid, or one which is replicated with the host chromosome, such as a bacteriophage. Examples of suitable vectors which have been widely employed are pBR322 and related vectors as well as pUC vectors and the like. Examples of suitable bacteriophages include M13 and lambda phage.

The organism harboring the vector carrying the DNA fragment or part thereof may be any organism which is capable of expressing said DNA fragment. The organism can be a microorganism such as a bacterium. Gram-positive as well as gram-negative bacteria may be employed. Especially a gram-negative bacterium such as E. coli is useful, but also gram-positive bacteria such as B. subtilis and other types of microorganisms such as yeasts or fungi or other organisms conventionally used to produce recombinant DNA products may be used. Another type of organism which may be used to express lactoferrin or a portion thereof is a higher eukaryotic organism or cell, including a plant and mammal cell. However, also higher organisms such as animals, e.g. sheep, cattle, goats, pigs, horses and domestic animals, including cats and dogs, are contemplated to be useful as host organisms for the production of lactoferrin or a part thereof.

When a higher organism, e.g. an animal, is employed for the production of lactoferrin or a part thereof, conventional transgenic techniques may be employed. These techniques comprise inserting the DNA fragment or one or more parts thereof into the genome of the animal in such a position that lactoferrin or part thereof is expressed together with a polypeptide which is inherently expressed by the animal, in many cases, a polypeptide which is easily recovered from the animal, e.g. a polypeptide which is secreted by the animal, such as in milk, colostrum, bile, sweat, tears, saliva or the like. Alternatively, the DNA fragment could be inserted into the genome of the animal in a position allowing the gene product of the expressed DNA sequence to be retained in the animal body so that a substantial steady immunization of the animal takes place. When a microorganism is used for expressing the DNA fragment, the cultivation conditions will typically depend on the type of microorganism employed, and the skilled art worker will know which cultivation method to choose and how to optimize this method.

The production of lactoferrin proteins or a part thereof by recombinant techniques has a number of advantages: it is possible to produce lactoferrin or lactoferrin fusion protein or a polypeptide part thereof by culturing non-pathogenic organisms or other organisms which do not affect the immunological properties of the lactoferrin or lactoferrin fusion protein or a polypeptide part thereof, it is possible to produce the protein in higher quantities than those obtained when recovering lactoferrin proteins from any wild type fractions, and it is possible to produce parts of lactoferrin proteins which may not be isolated and/or purified from native source. The higher quantities of lactoferrin or lactoferrin fusion protein or a polypeptide part thereof may for instance be obtained by using high copy number vectors for cloning the DNA fragment or by using a strong promoter to induce a higher level of expression than the expression level obtained with the promoters P1 and P2 present on the DNA fragment disclosed herein. By use of recombinant DNA techniques for producing the lactoferrin protein or lactoferrin fusion protein or a polypeptide part thereof, unlimited amounts of a substantially pure protein or polypeptide which is not “contaminated” with other components which are normally present in isolates from the native organism may be obtained. Thus, it is possible to obtain a substantially pure lactoferrin protein or lactoferrin fusion protein or a polypeptide part thereof which is not admixed with other proteins from the native source which can have an adverse effect when present in a pharmaceutical composition in which the lactoferrin is an intended constituent. A substantially pure lactoferrin or lactoferrin fusion protein or a polypeptide part thereof has the additional advantage that the exact concentration thereof in a given pharmaceutical preparation is known so that an exact dosage may be administered to the individual to be treated. An important aspect of the present disclosure concerns methods of making and using a pharmaceutical composition for treatment of an autoimmune disease, condition or disorder in an animal, such as a mammal, including a human being, which composition comprises an immunologically effective amount of any one of the above defined proteins, polypeptides or fractions or combinations thereof together with a pharmaceutically acceptable carrier or vehicle. It should be understood that the term “animal” includes the human animal.

As used herein, the term “purifying” is used interchangeably with the term “isolating” and generally refers to any separation of a particular component from other components of the environment in which it is found or produced. For example, purifying a recombinant protein from plant cells in which it was produced typically means subjecting transgenic protein-containing plant material to separation techniques such as sedimentation, centrifugation, filtration, and chromatography. The results of any such purifying or isolating step(s) may still contain other components as long as the results have less of the other components (“contaminating components”) than before such purifying or isolating step(s).

The compounds of the present disclosure can be purified or “at least partially purified” by art-known techniques such as reverse phase chromatography high performance liquid chromatography, ion exchange chromatography, gel electrophoresis, affinity chromatography and the like. The actual conditions used to purify a particular compound will depend, in part, on synthesis strategy and on factors such as net charge, hydrophobicity, hydrophilicity, etc., and will be apparent to those having skill in the art.

Compounds useful in the invention include those described herein in any of their pharmaceutically acceptable forms, including isomers such as diastereomers and enantiomers, salts, solvates, and polymorphs, as well as racemic mixtures and pure isomers of the compounds described herein, where applicable.

The terms “subject”, “individual” or “patient” are used interchangeably herein and refer to a vertebrate, preferably a mammal. Mammals include, but are not limited to, humans.

“Pharmaceutically acceptable excipient or carrier” refers to an excipient that may optionally be included in the compositions of the invention and that causes no significant adverse toxicological effects to the patient. In particular, in the present instance, such refers to an excipient that can be taken into the mammalian subject's body in association with an active compound (here lactoferrin) with no significant adverse toxicological effects to the subject.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly salts containing pharmaceutically acceptable cations include, but are not limited to, sodium, potassium, calcium, aluminum, lithium, and ammonium (including substituted ammonium).

“Optional” or “optionally” means that the subsequently described circumstance may or may not be present or occur, so that the description includes instances where the circumstance is present or occurs and instances where it is not present or does not occur.

“Substantially absent” or “substantially free” of a certain feature or entity means nearly totally or completely absent the feature or entity. For example, for a subject administered lactoferrin, the substantial absence of an observable side effect means that such side effect is either non-detectable, or occurs only to a negligible degree, e.g., to an extent or frequency that is reduced by about 50% or more when compared to either the frequency or intensity of the same side effect observed in an untreated patient.

The terms “pharmacologically effective amount” or “therapeutically effective amount” as related to the present composition refer to a non-toxic but sufficient amount of the active agent (or composition containing the active agent) to provide the desired level of active agent in the bloodstream or at the site of action (e.g. intracellularly) in the subject to be treated, to provide a desired physiological, biophysical, biochemical, pharmacological or therapeutic response, such as amelioration of the manifestations of the autoimmune disease or disorder. The exact amount required will vary from subject to subject, and will depend on numerous factors, such as the active agent, the activity of the composition, the delivery device employed, the physical characteristics of the composition, intended patient use (i.e., the number of doses administered per day), as well as patient considerations, such as species, age, and general condition of the subject, the severity of the condition being treated, additional drugs being taken by the subject, mode of administration, and the like. These factors and considerations can readily be determined by one skilled in the art, based upon the information provided herein. An appropriate “effective” amount in any individual case may be determined by one of ordinary skill in the art using routine experimentation, based upon the information provided herein.

Patients can be stratified and selected based on age; for example, in some embodiments, a pediatric population is excluded, and an adolescent and/or adult population is selected for treatment by the disclosed methods. Patients may also be stratified by gender. IBD is equally prevalent in either gender in the pediatric population. However, most North American studies show that ulcerative colitis (UC) is more common in men than in women. In addition, men are more likely than women to be diagnosed with ulcerative colitis in their 50s and 60s. Patients also can be stratified according to the severity of their symptoms, often described as mild-moderate, or moderate to severe.

The term “about,” particularly in reference to a given quantity, is meant to encompass deviations of plus or minus five percent.

As used herein, the terms “protein,” “polypeptide,” “oligopeptide” and “peptide” have their conventional meaning and are used interchangeably to denote a polymer of at least two amino acids covalently linked by an amide bond, regardless of length or post-translational modification (e.g., glycosylation, phosphorylation, lipidation, myristilation, ubiquitination, etc.). Furthermore, the polypeptides described herein are not limited to a specific length. Included within this definition are D- and L-amino acids, and mixtures of D- and L-amino acids. This term also does not refer to or exclude post-expression modifications of the polypeptide, for example, glycosylations, acetylations, phosphorylations and the like, as well as other modifications known in the art, both naturally occurring and non-naturally occurring. A polypeptide may be an entire protein, or a subsequence thereof. Polypeptides can also refer to amino acid subsequences comprising epitopes, i.e., antigenic determinants substantially responsible for the immunogenic properties of a polypeptide and being capable of evoking an immune response.

“Fusion polypeptide:” In one embodiment of the invention, the extension has a sequence that corresponds to a sequence of a signal peptide capable of effecting transport across membranes, such that the compound is a “fusion polypeptide.” Such fusion polypeptides are particularly advantageous for administering to cells compounds of the invention that may not readily traverse cell membranes. The signal sequence may be fused to either the N-terminal or C-terminal portion of the compound, depending upon the characteristics of the particular signal sequence selected. Signal sequences capable of transporting molecules into cells are well-known in the art. Any of these sequences may be used in connection with the compounds of the invention. Specific examples of such sequences include HIV Tat sequences (see, e.g., Fawell et al., 1994, Proc. Natl. Acad. Sci. USA 91:664; Frankel et al., 1988, Cell 55:1189; Savion et al., 1981, 1 Biol. Chem. 256:1149; Derossi et al., 1994, J Biol. Chem. 269:10444; Baldin et al., 1990, EMBO J. 9:1511; U.S. Pat. Nos. 5,804,604; 5,670,617; and 5,652,122, the disclosures of which are incorporated herein by reference), antennapedia sequences (see, e.g., Garcia-Echeverria et al., 2001, Bioorg. Med. Chem. Lett. 11:1363-1366; Prochiantz, 1999, Ann. NY Acad. Sci. 886:172-179; Prochiantz, 1996, Curr. Opin. Neurobiol. 6:629-634; U.S. Pat. No. 6,080,724, and the references cited in all of the above, the disclosures of which are incorporated herein by reference) and poly(Arg) or poly(Lys) chains of 5-10 residues. Additional non-limiting examples of specific sequences can be found in U.S. Pat. Nos. 6,248,558; 6,043,339; 5,807,746 U.S. Pat. Nos. 6,251,398; 6,184,038 and 6,017,735, the disclosures of which are incorporated herein by reference.

“NH2 Terminal Modifications:” The terminus of the peptide compounds of the invention corresponding to the amino terminus, if present, may be in the “free” form (e.g., H₂N—), or alternatively may be acylated with a group of the formula R²C(O)— or R²S(O)₂—, wherein R² is as previously defined. In one embodiment, R² is selected from the group consisting of (C₁-C₆) alkyl, (C₅-C₁₀) aryl, (C₆-C₁₆) arylalkyl, 5-10 membered heteroaryl or 6-16 membered heteroarylalkyl.

In another embodiment, the amino terminus may be “blocked” with a blocking group designed to impart the compound with specified properties, such as a low antigenicity. Non-limiting examples of such blocking groups include polyalkylene oxide polymers such as polyethylene glycol (PEG). A variety of polymers useful for imparting compounds, and in particular peptides and proteins, with specified properties are known in the art, as are chemistries suitable for attaching such polymers to the compounds. Specific non-limiting examples may be found in U.S. Pat. Nos. 5,643,575; 5,730,990; 5,902,588; 5,919,455; 6,113,906; 6,153,655; and 6,177,087, the disclosures of which are incorporated herein by reference.

“Carboxy Terminus Modifications:” The terminus of the peptide compounds corresponding to the C-terminus, if present, may be in the form of an underivatized carboxyl group, either as the free acid or as a salt, such as a sodium, potassium, calcium, magnesium salt or other salt of an inorganic or organic ion, or may be in the form of a derivatized carboxyl, such as an ester, thioester or amide. Such derivatized forms of the compounds may be prepared by reacting a compound having a carboxyl terminus with an appropriate alcohol, thiol or amine. Suitable alcohols, thiols or amines include, by way of example and not limitation, alcohols of the formula R²OH, thiols of the formula R²SH and amines of the formula R²NH₂, R²R²NH or NH₃, where each R² is, independently of the others, as previously defined.

“L or D form amino acids:” As will be recognized by skilled artisans, the various X′ residues comprising the compounds of the invention may be in either the L- or D-configuration about their C_{α} carbons. In one embodiment, all of the C_{α} carbons of a particular compound are in the same configuration. In some embodiments of the invention, the compounds comprise specific chiralities about one or more C_{α} carbon(s) and/or include non-peptide linkages at specified locations so as to impart the compound with specified properties. For example, it is well-known that peptides composed in whole or in part of D-amino acids are more resistant to proteases than their corresponding L-peptide counterparts. Thus, in one embodiment, the compounds are peptides composed in whole or in part of D-amino acids. Alternatively, compounds having good stability against proteases may include peptide analogs including peptide linkages of reversed polarity at specified positions. For example, compounds having stability against tryptic-like proteases include peptide analogs having peptide linkages of reversed polarity before each L-Arg or L-Lys residue; compounds having stability against chymotrypsin-like proteases include peptide analogs having peptide linkages of reversed polarity before each small and medium-sized L-aliphatic residue or L-non-polar residue. In another embodiment, compounds having stability against proteases include peptide analogs composed wholly of peptide bonds of reversed polarity. Other embodiments having stability against proteases will be apparent to those of skill in the art. Additional specific embodiments of the compounds are described below.

“Unnatural” or “non-natural” amino acids are non-proteinogenic amino acids that either occur naturally or are chemically synthesized. Whether utilized as building blocks, conformational constraints, molecular scaffolds or pharmacologically active products, unnatural amino acids represent a nearly infinite array of diverse structural elements for the development of new leads in peptidic and non-peptidic compounds. Due to their seemingly unlimited structural diversity and functional versatility, they are widely used as chiral building blocks and molecular scaffolds in constructing combinatorial libraries. Drug discovery has benefited from novel, short-chain peptide ligand mimetics (peptidomimetics) with both enhanced biological activity and proteolytic resistance. Used as molecular probes, they can help to better understand the function of biological systems. Optimized and fine-tuned analogues of peptidic substrates, inhibitors or effectors are also excellent analytical tools and molecular probes for investigating signal transduction pathways or gene regulation.

Exemplary non-natural amino acids that may be used in the compositions disclosed herein include: β-amino acids (β³ and β²), homo-amino acids, proline and pyruvic acid derivatives, 3-substituted Alanine derivatives, Glycine derivatives, ring-substituted Phenylalanine and Tyrosine derivatives, linear core amino acids, N-methyl amino acids, etc. (for additional examples, sigmaaldrich.com).

The term “prevention,” “amelioration” or “treatment” of and autoimmune disease or disorder refers to any indicia of success in the treatment of a pathology or condition, including any objective or subjective parameter such as abatement, remission or diminishing of symptoms or an improvement in a patient's physical or mental well-being. Amelioration of symptoms can be based on objective or subjective parameters; including the results of a physical examination and/or a psychiatric evaluation.

The lactoferrin composition described herein will generally be used in an amount effective to treat, ameliorate or prevent the particular autoimmune disease or disorder in the subject being treated. The lactoferrin composition may be administered therapeutically to achieve therapeutic benefit or prophylactically to achieve prophylactic benefit. By therapeutic benefit is meant eradication or amelioration of the underlying disorder being treated, e.g., eradication or amelioration of the underlying autoimmune disease or disorder, and/or eradication or amelioration of one or more of the symptoms associated with the underlying autoimmune disease or disorder such that the patient reports an improvement in feeling or condition, notwithstanding that the patient may still be afflicted with the underlying disorder. For example, administration of the lactoferrin composition to a patient suffering from an autoimmune disease provides therapeutic benefit not only when the underlying autoimmune disease response is eradicated or ameliorated, but also when the patient reports a decrease in the severity or duration of the symptoms associated with the autoimmune disease. Therapeutic benefit also includes halting or slowing the progression of the disease, regardless of whether improvement is realized.

For prophylactic administration, the lactoferrin composition may be administered to a patient at risk of developing an autoimmune disease or disorder. For example, prophylactic administration of the lactoferrin composition may be used to avoid the onset of symptoms in a patient diagnosed with the underlying disorder. Similarly, because some autoimmune disorders are associated with a prior viral infection, for example, the lactoferrin composition may be administered prophylactically to healthy individuals who are at risk of exposure to a virus or other infectious agent associated with subsequent development of the autoimmune disease or disorder to prevent the onset of the disorder. For example, the lactoferrin composition may be administered to a healthy adolescent prior to infection with a virus to avoid development of the autoimmune disease or disorder.

The amount of lactoferrin composition administered will depend upon a variety of factors, including, for example, the particular indication being treated, the mode of administration, whether the desired benefit is prophylactic or therapeutic, the severity of the indication being treated and the age and weight of the subject animal/patient, the bioavailability of the particular lactoferrin and other active ingredient(s) in the composition, etc. Determination of an effective dosage is well within the capabilities of those skilled in the art. Initial dosages may be estimated initially from in vitro assays.

The one or more lactoferrin proteins can be further formulated together with one or more pharmaceutically acceptable excipients to produce a pharmaceutical composition. The term “excipient” or “vehicle” as used herein means any substance, not itself a therapeutic agent, used as a carrier for delivery of a therapeutic agent and suitable for administration to a subject, e.g. a mammal or added to a pharmaceutical composition to improve its handling or storage properties or to permit or facilitate formation of a dose unit of the composition into a discrete article such as a capsule or tablet suitable for oral administration. Excipients and vehicles include any such materials known in the art, e.g., any liquid, gel, solvent, liquid diluent, solubilizer, or the like, which is nontoxic and which does not interact with other components of the composition in a deleterious manner. Administration can mean oral administration, inhalation, enteral administration, feeding or inoculation by intravenous injection. The excipients may include standard pharmaceutical excipients, and may also include any components that may be used to prepare foods and beverages for human and/or animal consumption, feed or bait formulations or other foodstuffs.

For example, excipients include, by way of illustration and not limitation, diluents, disintegrants, binding agents, adhesives, wetting agents, lubricants, glidants, crystallization inhibitors, surface modifying agents, substances added to mask or counteract a disagreeable taste or odor, flavors, dyes, fragrances, and substances added to improve appearance of the composition. Excipients employed in compositions of the disclosure can be solids, semi-solids, liquids or combinations thereof. Compositions of the disclosure containing excipients can be prepared by any known technique of pharmacy that comprises admixing an excipient with a drug or therapeutic agent. Other excipients such as colorants, flavors, and sweeteners, which may make the oral formulations of the present disclosure more desirable to the subject being treated can also be used in compositions of the present disclosure.

“Permeant,” “drug,” or “pharmacologically active agent” or any other similar term means any chemical or biological material or compound, inclusive of peptides, suitable for transmucosal administration by the methods previously known in the art and/or by the methods taught in the present disclosure, that induces a desired biological or pharmacological effect, which may include but is not limited to (1) having a prophylactic effect on the organism and preventing an undesired biological effect such as preventing an infection, (2) alleviating a condition caused by a disease, for example, alleviating pain or inflammation caused as a result of disease, and/or (3) either alleviating, reducing, or completely eliminating the disease from the organism. The effect may be local, such as providing for a local anaesthetic effect, or it may be systemic. This disclosure is not drawn to novel permeants or to new classes of active agents. Rather it is limited to the mode of delivery of agents or permeants which exist in the state of the art or which may later be established as active agents and which are suitable for delivery by the present disclosure. Such substances include broad classes of compounds normally delivered into the body, including through body surfaces and membranes, including skin. In general, this includes but is not limited to: antiinfectives such as antibiotics and antiviral agents; analgesics and analgesic combinations; anorexics; antihelminthics; antiarthritics; antiasthmatic agents; anticonvulsants; antidepressants; Antidiabetic agents; antidiarrheals; antihistamines; antiinflammatory agents; antimigraine preparations; antinauseants; antineoplastics; antiparkinsonism drugs; antipruritics; antipsychotics; antipyretics; antispasmodics; anticholinergics; sympathomimetics; xanthine derivatives; cardiovascular preparations including potassium and calcium channel blockers, beta-blockers, alpha-blockers, and antiarrhythmics; antihypertensives; diuretics and antidiuretics; vasodilators including general coronary, peripheral and cerebral; central nervous system stimulants; vasoconstrictors; cough and cold preparations, including decongestants; hormones such as estradiol and other steroids, including corticosteroids; hypnotics; immunosuppressives; muscle relaxants; parasympatholytics; psychostimulants; sedatives; and tranquilizers. By the method of the present disclosure, both ionized and nonionized drugs may be delivered, as can drugs of either high or low molecular weight.

“Buccal” drug delivery is meant delivery of a drug by passage of a drug through the buccal mucosa into the bloodstream. Buccal drug delivery may be effected herein by placing the buccal dosage unit on the upper gum or opposing inner lip area of the individual undergoing drug therapy.

According to one aspect of the disclosure, the compositions and/or formulations can include more than one type of protein and/or active agent for the treatment of autoimmune diseases or disorders.

The oral formulations according to the present disclosure can be prepared in any manner suitable to deliver the lactoferrin protein(s) in order to induce an immune response in the organism to which the formulation is administered. Conventional blending, tableting, and encapsulation techniques known in the art can be employed. Oral dosage forms are suitable for administering the one or more lactoferrin protein(s) produced in accordance with the present disclosure due to their ease of administration; however, parenteral formulations containing the recombinant lactoferrin protein(s) of the present disclosure are also envisioned and these may be prepared in accordance with known methods. Examples of dosage forms for administration to a human include a tablet, a caplet, a hard or soft capsule, a lozenge, a cachet, a dispensable powder, granules, a suspension or solution, an elixir, a liquid, or any other form reasonably adapted for oral administration. Examples of dosage forms for administration to an animal include foods, liquids, baits, and any other compositions that are likely to be consumed by the subject to be treated.

When oral formulations are prepared from a genetically-modified monocot seed, it is possible to first purify the recombinant lactoferrin protein, and then incorporate it into a food or beverage formulation. In accordance with this aspect of the disclosure, any components that are added to the genetically-modified monocot seed to form a food or beverage formulation may be considered excipients. One of the benefits of the present disclosure is the ability to directly utilize the genetically-modified monocot seed in the production of such a food or beverage formulation without first purifying the lactoferrin protein. This is possible at least in part because of the relatively high levels of the recombinant lactoferrin protein in the seeds produced by the methods described herein and known in the art.

The oral formulations containing lactoferrin protein according to the present disclosure may be administered in any dose adequate to modulate a T cell response in the subject. In one embodiment of the present disclosure, the oral formulation is administered in doses of from about 0.1 microgram (μg)/day to about 100 mg/day, about 1 μg/day to about 10 mg/day, about 5 μg/day to about 5 mg/day, about 10 μg/day to about 1 mg/day, or about 25 μg/day to about 0.5 mg/day. In some embodiments, the oral formulation is administered in a dose of 50 mg/kgbw/day to about 2 g/kgbw/day. In some embodiments, the oral formulation is administered in a dose of about 100 mg/kgbw/day to about 10 grams/kgbw/day. In some embodiments, the oral formulation is administered in a dose of 500 mg/kgbw/day to about 5 g/kgbw/day. In some embodiments, the oral formulation is administered in a dose of about 100 mg/kgbw/day to about 2 grams/kgbw/day. In some embodiments, the oral formulation is administered in a dose of about 50 mg/kgbw/day. In some embodiments, the oral formulation is administered in a dose of about 100 mg/kgbw/day. In some embodiments, the oral formulation is administered in a dose of about 200 mg/kgbw/day. In some embodiments, the oral formulation is administered in a dose of about 500 mg/kgbw/day. In some embodiments, the oral formulation is administered in a dose of about 1000 mg/kgbw/day.

According to some embodiments, it is also possible to prepare parenterally-administered compositions for treatment of autoimmune disease or allergy using the recombinant lactoferrin produced in monocot seeds by first purifying the lactoferrin protein(s) from monocot seeds, and then incorporating them into a standard parenteral pharmaceutical formulation using techniques known in the art. Such parenteral formulations may be administered in any amount sufficient to confer treatment or amelioration of symptoms of the autoimmune disease or allergy.

For example, to help the release of lactoferrin protein in small intestine, the oral formulations may be tableted or pelleted, or encapsulated, and may be enteric-coated. Enteric coating prevents a tablet or capsule from dissolving before it reaches the small intestine. Alternatively the material may be spheronized into microparticles and may be enterically coated. Spheroids may be produced in the size range of 250 μm to 850 μm. Enteric coatings are known to be selectively insoluble substances that do not dissolve in the acidic environment of the stomach, but dissolve in the higher pH of the small intestine, resulting in a specific release of lactoferrin protein(s) in the small intestine.

The lactoferrin composition described herein will provide therapeutic or prophylactic benefit without causing substantial toxicity. Any potential toxicity of the lactoferrin composition may be identified/determined using standard pharmaceutical procedures. The dose ratio between toxic and therapeutic (or prophylactic) effect is the therapeutic index. Lactoferrin composition that exhibit high therapeutic indices are desirable.

The lactoferrin or lactoferrin fusion protein or a polypeptide part thereof may be prepared by recombinant DNA techniques or by solid or liquid phase peptide synthesis. Polypeptides prepared in this manner are especially desirable as components of the presently described pharmaceutical compositions, as these polypeptides are essentially free from other contaminating components which will influence the therapeutic properties of the polypeptides. Thus, polypeptides prepared by recombinant DNA techniques or by solid or liquid phase peptide synthesis may be obtained in a substantially pure form which is very desirable for pharmaceutical compositions. When proteins or other immunogenically active components are present in any of the purification fractions are employed as vaccine constituents, these may advantageously be recovered from the fractions by any conventional method, e.g. a method in which antibodies, such as monoclonal antibodies, reactive with the proteins or other immunologically active components of fractions are immobilized to a matrix, the matrix is contacted with the fraction in question, washed, and finally the antigen-antibody complex fixed to the matrix is treated so as to release the lactoferrin protein, fragment or variant in a purified form. The lactoferrin protein may also be isolated by means of column affinity chromatography involving antibodies fixed to the column matrix.

“High affinity” for an IgG antibody refers to an antibody having a KD of 10⁻⁸ M or less; or 10⁻⁹ M or less; or 10⁻¹⁰ M or less. However, “high affinity” binding can vary for other antibody isotypes. For example, “high affinity” binding for an IgM isotype refers to an antibody having a KD of 10⁻⁷ M or less; or 10⁻⁸ M or less.

As will be understood by those of skill in the art, in some cases it may be advantageous to use a nucleotide sequences possessing non-naturally occurring codons. Codons preferred by a particular eukaryotic host can be selected, for example, to increase the rate of expression or to produce recombinant RNA transcripts having desirable properties, such as a longer half-life, than transcripts produced from naturally occurring sequence. As an example, it has been shown that codons for genes expressed in rice are rich in guanine (G) or cytosine (C) in the third codon position (Huang et al., (1990) J. CAASS 1: 73-86). Changing low G+C content to a high G+C content has been found to increase the expression levels of foreign protein genes in barley grains (Horvath et al., (2000) Proc. Natl. Acad. Sci. USA 97: 1914-19). If a rice plant is selected, the genes employed in the present disclosure may be based on the rice gene codon bias (Huang et al., supra) along with the appropriate restriction sites for gene cloning. These codon-optimized genes may be linked to regulatory and secretion sequences for seed-directed expression and these chimeric genes then inserted into the appropriate plant transformation vectors.

Administration of lactoferrin compositions of the present disclosure may also be in the form of a flour, meal, pellets, seeds, grains, liquid suspension or extract, or lyophilized powder to be added to food. Specifically contemplated is each and every combination and permutation of these. The lactoferrin composition may be significantly-, fully- or partially-purified from the original mammalian source or from the source of recombinant heterologous expression. The lactoferrin composition may comprise 100% or nearly 100% lactoferrin (±10%), or may comprise lactoferrin in combination with other milk proteins, or sugars (e.g., glucose) and/or other nutrients, foodstuffs, stabilizers, excipients, capsule protectants, etc.

Expression of human lactoferrin in monocot plants is an attractive approach to producing high quality hLF free from any mammalian, viral or bacterial contaminants. Using monocot grains such as rice is also advantageous due to their good nutritional value and low allergenicity, and any residual materials from rice introduce no risk. To produce human lactoferrin and lysozyme in large quantities, recombinant technology has been used with rice as the host organism (Huang, et al., 2002, Mol. Breed. 10:83-94; Nandi, et al., 2002, Plant Sci., 163:713-722). The genes for human milk lactoferrin and lysozyme have been inserted into transgenic rice, and the recombinant proteins produced were tested extensively in the laboratory and found to be substantially equivalent to the native proteins in all biochemical and functional tests. Specifically, the recombinant form of lysozyme was identical to native lysozyme because this protein is not posttranslationally modified (i.e., no glycosylation or phosphorylation). The lactoferrin protein was identical to native human lactoferrin; however, because lactoferrin is a glycosylated protein, the glycans attached to the protein backbone are of rice origin (Nandi, et al., 2005, Trans. Res. 14:237-249). Thus, some terminal carbohydrate residues consist of xylose, which is not the case in human proteins, and sialic acid, a common terminal residue in native human lactoferrin, is lacking. Recombinant human lactoferrin produced in rice is functionally equivalent to native human lactoferrin with regard to iron-binding, anti-microbial activity and receptor binding (Nandi, et al., 2002, Plant Sci., 163:713-722). Although not all of the functions of lactoferrin may be related to its intestinal receptor (Lönnerdal B. and Iyer S., 1995, Annu. Rev. Nutr. 15:93-110), it is evident that absence of glycans, differences in glycosylation pattern, and composition do not affect receptor binding (Suzuki, et al., 2003, J. Pediatr. Gastroenterol. Nutr. 36:190-199). Thus, it is highly likely that recombinant human lactoferrin, even with a somewhat different glycosylation pattern than native human lactoferrin, has the same biological function in vivo. Addition of recombinant human lactoferrin and lysozyme to a rice-based oral rehydration solution had beneficial effects on children with acute diarrhea and associated dehydration (Zavaleta, et al., 2007, J. Pediatr. Gastroenterol. Nutr. 44:258-64).

### II. ETIOLOGY OF AUTOIMMUNITY

Autoimmune disease arises from the dysregulated immune response toward a self-antigen. This aberrant activation results in an overproduction of pro-inflammatory cytokines such as TNF by monocytes, macrophages, and T cells, thereby allowing for persistence of a hyperactivated immune response and subsequent pathology (Naser, et al., 2011, Clin. Vaccine Immunol. 18:1416-9; Van Deventer, S. J., 1997, Gut 40:443-8). The contribution of CD4⁺ T cells has been identified as being an important driver of pathogenesis in numerous autoimmune conditions, whereby an increase in activation and populations of CD4⁺ and CD8⁺ T cells is observed (Funderburg, et al., 2013, Immunology 140:87-97). More recently, it has been appreciated that an over-activation of Th1/Th17 phenotypes drives much of the chronic pathology in autoimmunity (Zenewicz, et al., 2009, Trends Mol. Med. 15:199-207).

On the other hand, it has been observed that the activity of pro-inflammatory T cells can be tempered by the action of a subset of CD4⁺ regulatory T cells (Treg). Classically defined Tregs are found within the CD4⁺ T-cell pool and are identified by their constitutive expression of Foxp3, and the IL-2 receptor α-chain (CD25) (Rudensky A. Y., 2011, Immunol. Rev. 241:260-8). In fact, regulatory T cells were first identified by their elevated expression of the high-affinity IL-2 receptor CD25 (IL-2Rα). Mice lacking IL-2 signaling via antibody neutralization or genetic deficiency of IL-2 or IL-2 receptors show Treg deficiencies and spontaneous autoimmune disease including IBD. Pioneering studies by Powrie et al. (Powrie, et al., 1993, Int. Immunol. 5:1461-71; Powrie, et al., 1994, J. Exp. Med. 179:589-600) demonstrated that the pathology in a mouse model of T-cell-induced colitis, which mimics human IBD, can indeed be prevented by adoptive transfer of Foxp3⁺ Tregs. Furthermore, Tregs can not only prevent but also cure IBD in mouse models (Mottet, et al., 2003, J. Immunol. 170:3939-43).

Underscoring the importance of Tregs in immune regulation, Foxp3 deficiency results in global failure of Treg cell development, leading to a lethal multi-system autoimmune disease immunodysregulation polyendocrinopathy enteropathy X-linked syndrome (IPEX) (Yong, et al., 2008, J. Clin. Immunol. 28:581-7; Carneiro-Sampaio, M. and Coutinho, A. 2015, Front. Immunol. 6:185). The most commonly affected organ in IPEX is the intestine, highlighting an important role for Treg cells in the gastrointestinal tract. In addition to IPEX, other genetic deficiencies suggest the importance of Treg in IBD. Mutations in WASP, CD25, and IL-10 all lead to abnormal Treg cell numbers and/or function, and also increase an individual's risk for autoimmune disease (Boden, E. K. and Snapper, S. B. 2008, Curr. Opin. Gastroenterol. 24:733-41).

Lactoferrin may be useful as a therapeutic, in suppression of inflammation and amelioration of autoimmune diseases and/or neurodegenerative diseases, including Parkinson's disease, Alzheimer's disease, cognitive decline in the elderly resulting from chronic inflammation, amyotrophic lateral sclerosis, multiple sclerosis, and HIV-associated inflammation and/or neurodegeneration. Lactoferrin might also be used in CAR T-cell therapy or adoptive cell therapy in the restoration of Treg number and function, in order to suppress inflammation, and/or slow disease progression in autoimmune diseases. For example, lactoferrin may be a useful addition to the methods of in vitro expansion of Tregs emplyoing anti-CD3/CD28 beads, interleukin (IL)-2 and rapamcyin reported by (Alsuliman, et al., Cytotherapy, August 2016, in press) or in CAR T-cell therapy, or in suppressing the overactivation of the immune system, and/or the toxicity of CAR T-cell therapy.

### III. ANTI-INFLAMMATORY EFFECTS OF LACTOFERRIN

Overall, rhLF is demonstrated herein to be a potent modulator of IL-10, comparable to lactoferrin purified from bovine or human sources. Lactoferrin skews T-cells to the Treg phenotype. The rhLF described herein can activate MAPK signaling in a similar manner to human LF. MAPK inhibition (most notably MEK inhibiton) results in shut-down of rhLF-induced IL-10 and IL-2. Furthermore, various routes of administration of rhLF results in Treg homing to the gut and its associated lymph tissues in healthy mice.

Lactoferrin (LF) is a single-chain iron-binding glycoprotein of approximately 80 kDa that belongs to the human family of transferrins (Brock J. H. 2002, Biochem. Cell Biol. 80:1-6). LF is present in myriad mucosal fluids (Laibe, et al., 2003, Clin. Chem. Lab. Med. 41:134-8; Ohashi, et al., 2003, Am. J. Ophthalmol. 136:291-9; Niemela, et al., 1989, Hum. Reprod. 4:99-101; Lin, et al., 2001, Oral Microbiol. Immunol. 16:270-8; Caccavo, et al., 1999, Int. J. Clin. Lab. Res. 29:30-5), but is most predominant in human milk, particularly in the colostrum during early lactation, where it has been suggested to promote the healthy growth and development of the GI tract (Zhang, et al., 2001, Adv. Exp. Med. Biol. 501:107-13), promote the growth of commensal bacterial populations and protect against the establishment of pathogenic bacteria and viruses (Barboza, et al., 2012, Mol. Cell. Proteomics 11:M111 015248; Ochoa, T. J. and Cleary, T. G., 2009, Biochimie 91:30-4; Ammendolia, et al., 2012, Pathog. Glob. Health 106:12-9). Human colostrums and mature breast milk contain 5.8 mg/mL and 3.3 mg/mL of LF, respectively (Montagne, et al., 1999, J. Pediatr. Gastroenterol. Nutr. 29:75-80; Montagne, et al., 2001, Adv. Exp. Med. Biol. 501:241-7). In contrast, bovine colostrum and milk contain markedly reduced concentrations of LF (1.5 mg/mL in colostral whey and 20-200 μg/mL in milk) (Steijns, et al., 2000, Br. J. Nutr., 84 Suppl. 1:S11-7). LF has been previously identified for its multifactorial and beneficial activities in several models of human health including inflammation (Mueller, et al., 2011, Curr. Med. Res. Opin. 27:793-7; Zavaleta, et al., 2007, J. Pediatr. Gastroenterol. Nutr. 44:258-64), wound healing (Lyons, et al., 2007, Am. J. Surg. 193:49-54), infectious diseases (Zavaleta, et al., 2007, J. Pediatr. Gastroenterol. Nutr. 44:258-64; King, et al., 2007, J. Pediatr. Gastroenterol. Nutr. 44:245-51; Ochoa, et al., 2008, Clin. Infect. Dis. 46:1881-3) and cancer (Parikh, et al., 2011, J. Clin. Oncol. 29:4129-36; Hayes, et al., 2010, Invest. New Drugs 28:156-62).

Oral administration of hLF has been shown to suppress a number of pro-inflammatory cytokines in numerous models of colitis and sepsis, including TNF, IL-1β and IL-12. In other studies of mucosal inflammation in mice, hLF has been shown to be more efficacious than bLF (Haversen, et al. 2000, Infect. Immun. 68:5816-23). In experimental models of sepsis, and rheumatoid arthritis, LF has been demonstrated to exert protection through inhibiting the production of pro-inflammatory cytokines, (TNF, IL-6 and IL-1β), and stimulating anti-inflammatory and pro-restitution cytokines (IL-10 and IL-4) (Kimber, et al., 2002, Biochem. Cell. Biol. 80:103-7; Togawa, et al., 2002, Am. J. Physiol. Gastrointest. Liver Physiol. 283:G187-95; Togawa, et al., 2002, J. Gastroenterol. Hepatol. 17:1291-8; Haversen et al., 2003, Scand. J. Immunol. 57:2-10; Hayashida et al., 2004, J. Vet. Med. Sci. 66:149-54; Machnicki et al., 1993, Int. J. Exp. Pathol. 74:433-9). The molecular mechanisms through which LF exerts its anti-inflammatory effects is not completely understood but appear in part to occur through the inhibition of nuclear factor kappa B (NFκB) signaling pathways, through inhibition of intracellular TNF receptor associated factor 6 (TRAF6) signaling (Inubushi, et al., 2012, J. Biol. Chem. 287:23527-36). Further studies have demonstrated the ability for LF to enter the nucleus of the cell where it directly interacts with the NFκB response elements of pro-inflammatory genes thereby preventing NFκB induced gene expression in human monocytic and endothelial cells (Haversen et al., 2002, Cell. Immunol. 220:83-95; Kim, et al., 2012, FEBS Lett. 586:229-34). Interestingly, in a study investigating the role of human LF in the monocytic leukemia cell line, THP-1, it was shown that human LF demonstrates moderate activation of NFκB in a TLR4-dependent mechanism through the action of the carbohydrate moieties decorating LF, whereby the protein backbone of LF inhibits LPS-mediated activation of TLR4 (Ando, et al., 2010, FEBS J. 277:2051-66).

Further evidence for the anti-inflammatory effects of LF have come with the observation that a 20 amino acid peptide derived from the N terminal of LF (LFP-20) inhibit LPS-induced MyD88/NF-κB and MyD88/MAPK signaling independent of direct interaction with LPS (Zong, et al., 2015, Dev. Comp. Immunol. 52:123-131). It has also been reported that bovine LF acts as a potent anti-inflammatory agent on monocytes by triggering a tolerogenic-like program during their differentiation into dendritic cells (DC) (Puddu et al., 2011, PLoS One 6:e22504).

The present studies identify a complex role for LF in immunomodulation which may represent the requirement for initial activation of the inflammatory response in orchestrated manner, together with the requirement for a dampening of inflammation in an effort to prevent a sustained pathophysiological outcome. As noted above, in a study by Akin et al., bovine LF orally administered at 200 mg day was shown to reduce the incidence of necrotizing enterocolitis (NEC) in preterm infants. Their studies reported fewer sepsis episodes in the bovine LF intervention group (4.4 vs. 17.3/1,000 patient days, p=0.007) with none developing NEC, though without statistical significance (Akin, et al., 2014, Am. J. Perinatal. 31:1111-20). LF was known to have antibacterial functions, and, in the aforementioned study, Akin was evaluating the possible effect of LF on sepsis a (bacterial pathogenic infection), rather than identifying any effect LF had on immune dysregulation or Treg involvement inflammation.

Some illustrative publications describe other compositions and methods which may or may not be useful in conjunction with the present disclosure; each of these is incorporated herein by reference in its entirety.

PCT Publication WO 2010/125565 and US Patent Publications 2011200610, 20120135007 and 2013164302 describe immunomodulatory compositions comprising mammalian colostrum-derived immunoglobulin preparations for treating immune-related disorders, including preparations enriched with anti-LPS antibodies derived from mammalian colostrum or avian eggs, and optionally further antibodies against disease-associated antigens, colostrums, milk or milk product components and any adjuvants for treating, delaying or preventing the progression of a pathologic disorder such as chronic liver disease, cirrhosis and any complication or disorder associated therewith. In one embodiment, a composition that modulates regulatory T cells leading to modulation of the Th1/Th2, Tr1/Th3 cell balance toward an anti-inflammatory Th2, Tr1/Th3 immune response or a pro-inflammatory Th1 immune response thereby inhibiting or activating an immune response specifically directed toward said disorder is described. According to another optional embodiment, compositions comprising a combination of anti-LPS enriched immunoglobulin preparation with at least one colostrum-derived immunoglobulin preparation comprising immunoglobulins that recognize and bind at least one antigen specific for said pathologic disorder and thereby modulate immune-regulatory cells, specifically, regulatory T cells.

PCT Publications WO 2008/151449 and WO 2009/135306 describe uses of a dairy-derived composition useful in methods for treatment of eczema, and a colostrum-derived mixture enriched in growth factors, particularly, a bovine colostrum fraction in topical or oral formulations for the treatment of skin injury, diseases, wounds or ulcers. In one particular embodiment, at least 70%, and preferably 80%, of the proteins are hydrosoluble. The proteins may be comprised, for example but not limited to, of between 0.1 to 30% (w/w) of lactoferrin. In some embodiments, whey protein-derived products and compositions are said to modulate immune function, and in some embodiments, the composition comprises at least 70% (w/w) of dairy derived proteins. The total concentration of proteins in the composition would normally be of at least 80% wherein at least 60% is β-lactoglobulin.

PCT Publications WO 2006/054908 and WO 2008/140335 describe compositions and methods of immune or haematological enhancement, inhibiting tumour formation or growth, and treating or preventing cancer, and in particular, administration of milk fat or a milk fat analogue, optionally with at least one additional therapeutic factor, preferably lactoferrin or metal ion lactoferrin, preferably iron lactoferrin, preferably bovine lactoferrin, preferably iron bovine lactoferrin, or a metal ion functional variant or functional fragment thereof, to inhibit tumour formation or growth, maintain or improve one or more of the white blood cell count, the red blood cell count, or the myeloid cell count, reduce cachexia, mucositis, and anemia, stimulate the immune system and treat or prevent cancer and the symptoms of cancer and side-effects of cancer therapies. The methods and medicinal uses of the invention may be carried out by employing dietary (as foods or food supplements), nutraceutical or pharmaceutical compositions.

PCT Publication WO 2012/057636 describes the use of lactic acid whey or a derivative thereof for modulation of Th-1 and Th-2 immune response in a subject in need thereof. Native lactic whey is described as predominantly water and lactose, but also contains the majority of the milk serum proteins, those milk proteins that are not precipitated at pH 4.6 (i.e. the non-casein proteins) comprising α-lactalbumin, β-lactoglobulin, bovine serum albumins (BSA), immunoglobulins, lactoferrin, lactoperoxidase, lysozyme, etc.

U.S. Pat. Nos. 7,176,278 and 8,129,504 and PCT Publications WO 01/46254, WO 2003/020746, WO 2004/020405, WO 2004/019872 and WO 2004/020454 describe transferrin polynucleotides, polypeptides, antibodies and modified transferrin fusion proteins. Also described is melanotransferrin, which is said to possess high sequence homology with human serum transferrin, human lactoferrin and chicken transferrin. In one embodiment, the transferrin portion of the transferrin fusion protein includes a lactoferrin splice variant. In one example, a human serum lactoferrin splice variant can be a novel splice variant of a neutrophil lactoferrin. In one specific embodiment, the neutrophil lactoferrin splice variant can be that of Genbank Accession AAA59479 (protein) or GenBank Accession No. AY360320.1, a nucleotide sequence encoding a neutrophil lactoferrin (SEQ ID NO: 7). In some embodiments, the human neutrophil lactoferrin splice variant has the amino acid sequence specified in GenBank AAR12276.1; identified herein as SEQ ID NO: 9.

In another specific embodiment, the neutrophil lactoferrin splice variant can comprise the following amino acid sequence EDCIALKGEADA (SEQ ID NO: 8), which includes the novel region of splice-variance.

PCT Patent Publication WO 2010/062663 and US Patent Publication 2011212104 describe inflammatory bowel disease (IBD) biomarkers and methods of using the biomarkers (individually, or in groups) for diagnosing, assessing and monitoring disease progression or to monitor a course of therapy, confirm therapeutic efficacy, and/or to inform modifications of a therapeutic regimen. IL-17 and lactoferrin are described as biomarkers for IBD, and fecal lactoferrin is presented as a biomarker for monitoring treatment of IBD with the antiTNF-alpha antibody, infliximab. Particularly described is treatment of IBD with an IL-23 antagonist. IL-23 is said to be a key cytokine contributing to development and maintenance of Th17 cells, and mutation in IL-23R was associated with IBD.

PCT Patent Publication WO 2012/103324 and US Patent Publication 2012196299 describe methods, assays, and an apparatus for testing antigens associated with intestinal and/or blood-brain barrier permeability. This publication presents case studies in which patients are treated for celiac disease, intestinal barrier dysfunction/leaky gut syndrome, or multiple sclerosis, gut and blood-brain barrier (BBB) permeability issues, and possible neurological and/or autoimmune disorders by implementing a lectin-free and/or gluten-free diet and using lactoferrin as one of several probiotics given “for repairing the damaged BBB and gut barriers.”

None of the aforementioned patents or publications specifically describes the use of lactoferrin compositions in methods of modulating T cell subtypes. In spite of numerous publications providing evidence indicating that colostrum and/or milk protein compositions may be useful in methods of treatment in a multitude of inflammatory-based disease models, to date, the efficacy of LF in modulating a balance of T cell subtypes such that the inflammatory burden is reduced and the overactive autoimmune response is tempered by upregulating genes responsible for generation of Treg, and concomitantly downregulating genes responsible for generation of IL-17. Thus, LF is useful in treating and/or ameliorating symptoms and physiological manifestations of autoimmune diseases and disorders.

### IV. PRODUCTION OF FUSION PROTEINS

The present disclosure also provides methods for producing a modified lactoferrin-fusion protein using recombinant nucleic acid technologies well-known in the art. In general terms, the production of a recombinant form of a protein typically involves the following steps: A nucleic acid molecule is first obtained that encodes a lactoferrin fusion protein, and the nucleic acid molecule is preferably placed in operable linkage with suitable control sequences to form an expression unit containing the protein open reading frame. The expression unit is used to transform a suitable host and the transformed host is cultured under conditions that allow the production of the recombinant protein. Optionally the recombinant protein is isolated from the medium or from the cells; recovery and purification of the protein may not be necessary in some instances where some impurities may be tolerated. Each of the foregoing steps can be accomplished in a variety of ways. For example, the construction of expression vectors that are operable in a variety of hosts is accomplished using appropriate replicons and control sequences. Control sequences, expression vectors, and transformation methods are dependent on the type of host cell used to express the gene and are otherwise known to persons skilled in the art. Suitable restriction sites can, if not normally available, be added to the ends of the coding sequence so as to provide an excisable gene to insert into these vectors. A skilled artisan can readily adapt any host/expression system known in the art for use with the nucleic acid molecules of the invention to produce a desired recombinant protein. Any expression system may be used, including yeast, bacterial, animal, plant, eukaryotic and prokaryotic systems. In some embodiments, yeast, mammalian cell culture and transgenic animal or plant production systems are preferred. In other embodiments, yeast systems or plant systems that reduce native glycosylation patterns, hyper-glycosylation or proteolytic activity may be used.

Methods which may be useful to the presently described methods and compositions are also described in U.S. Pat. Nos. 6,569,831; 6,991,824; 7,138,150; 7,354,902; 7,417,178; 7,718,851; 8,158,857; 8,334,254; 8,686,225 and 8,703,699; and US Patent Publications 20120088729 and, each of which is incorporated herein by reference in its entirety.

### V. LACTOFERRIN MODULATES THE POPULATIONS AND FUNCTION OF T CELL SUBTYPES RELEVANT IN AUTOIMMUNE-BASED PATHOGENESIS IN ADOLESCENCE AND ADULT POPULATIONS

The nucleic acid sequence of the genomic DNA encoding lactoferrin and the encoded amino acid sequence, are publicly available through sequence databases such as GenBank and UniProtKB/Swiss-Prot (See, for example, NCBI Reference Sequence: NM 002343.5 and protein sequence P02788.6). The nucleic acid sequence encoding human lactoferrin is identified herein as SEQ ID NO: 1. An amino acid sequence for human lactoferrin protein having 692 amino acid residues is available through NCBI (National Center for Biotechnology Information, U.S. National Library of Medicine) asAccession No. 1FCK_A GI: 13096519. The human lactoferrin amino acid sequence set forth as SEQ ID NO: 2 (below) has a single amino acid residue difference at position 14, where the instant sequence has a glutamine instead of the asparagine present in GI: 13096519.

Human lactoferrin amino acid sequence (herein identified as SEQ ID NO: 2):

GRRRRSVQWCAVSQPEATKCFQWQRNMRKVRGPPVSCIKRDSPIQCIQAI
AENRADAVTLDGGFIYEAGLAPYKLRPVAAEVYGTERQPRTHYYAVAVVK
KGGSFQLNELQGLKSCHTGLRRTAGWNVPIGTLRPFLNWTGPPEPIEAAV
ARFFSASCVPGADKGQFPNLCRLCAGTGENKCAFSSQEPYFSYSGAFKCL
RDGAGDVAFIRESTVFEDLSDEAERDEYELLCPDNTRKPVDKFKDCHLAR
VPSHAVVARSVNGKEDAIWNLLRQAQEKFGKDKSPKFQLFGSPSGQKDLL
FKDSAIGFSRVPPRIDSGLYLGSGYFTAIQNLRKSEEEVAARRARVVWCA
VGEQELRKCNQWSGLSEGSVTCSSASTTEDCIALVLKGEADAMSLDGGYV
YTAGKCGLVPVLAENYKSQQSSDPDPNCVDRPVEGYLAVAVVRRSDTSLT
WNSVKGKKSCHTAVDRTAGWNIPMGLLFNQTGSCKFDEYESQSCAPGSDP
RSNLCALCIGDEQGENKCVPNSNERYYGYTGAFRCLAENAGDVAFVKDVT
VLQNTDGNNNEAWAKDLKLADFALLCLDGKRKPVTEARSCHLAMAPNHAV
VSRMDKVERLKQVLLHQQAKFGRNGSDCPDKFCLFQSETKNLLFNDNTEC
LARLHGKTTYEKYLGPQYVAGITNLKKCSTSPLLEACEFLRK

The role of lactoferrin in the immune system has been summarized in a recent review article (Siqueiros-Cendon, et al., 2014, Acta Pharmacol. Sin. 35(5):557-66) and will be briefly set forth hereinbelow. Three types of Antigen Presenting Cells (APCs) are important for maintenance of tissue homeostasis and the innate immune response via the major histocompatibility complex II (MHC II): (i) the macrophages (Mf), (ii) dendritic cells (DCs) and (iii) B-cells, which use specific surface receptors to capture foreign antigens and present their associated epitopes to T-cells.

The highly phagocytic Mfs play a central role in the control of infections, either by the direct intracellular killing of microorganisms or the secretion of cytokines to inhibit the replication of microorganisms. LF receptors are located on the surface of Mf in bovine and human models, and LF has been observed to increase the phagocytic activity of Mfs that are infected or have not yet been activated. Mfs are also involved in type II inflammation and tissue repair processes, and Mfs enable cross-talk between the innate and adaptive immune systems to stimulate antigen-specific T cells. LF also contributes to the suppression of pro-inflammatory cytokines and type I interferon (IFN α/β) induction, and it affects the ability of Mfs to present antigens for antigen-specific CD4+ T-cells in the adaptive immune system. IL-12, one of the major cytokines that are produced by Mfs, is a key modulator of IFNα. The main role of IL-12 at the site of infection is to recruit Mf, and it acts as a co-stimulator to maximize the secretion of IFNα from differentiated Th1 cells and memory T-cells. Up-regulation of adhesion molecules on the surface of the endothelium plays a key role in the recruitment and infiltration of leukocytes at inflammation sites. LF strongly inhibits TNF-α-stimulated expression of ICAM-1 by competing with NF-jB in endothelial cells, which suggests that LF reduces inflammatory events and the development of inflammatory diseases such as atherosclerosis.

Dendritic cells (DCs) are a heterogeneous population of functionally related phagocytic cells highly specialized for antigen recognition, and also play a key role in the immune system by controlling the induction of immunity and tolerance. DCs can manipulate T-cell differentiation and redirect memory T-cell functions. DCs play an important role in triggering T-cell responses that lead to the secretion of Th1 cytokines. It has also been shown that β-defensin 2, another key innate immunity molecule, act directly on DCs to induce their functional maturation and enable them to elicit a Th1 response. DCs possess LF receptors; bovine and human LF bind to the surface of peripheral blood-derived dendritic cells. LF has been proposed to play a role in the initiation of T-cell activation through the modulation of dendritic cell function; LF promotes antigen-specific delayed-type hypersensitivity (DTH) responses and activates bacillus Calmette-Guerin (Mycobacterium strain) (BCG)-specific T cells. The ability of dendritic cells to migrate upon antigen stimulation or capture is essential in the promotion of antigen-specific immune responses. LF acts as an alarmin to promote the recruitment and activation of APCs and antigen-specific immune responses. It has also been suggested to be a novel maturation factor for human dendritic cells. LF is a strong mediator of dendritic cell function. This observation, together with the above-described impact on Mfs, suggests that LF exerts its effect on cells involved in the commitment of pathogens (antigens) and can direct the development of adaptive immunity (Siqueiros-Cendon, et al., 2014, Acta Pharmacol. Sin. 35(5):557-66).

LF may be involved in immunomodulatory function (e.g., APC activation, maturation, migration and antigen presentation) and serve to bridge innate and adaptive cell functions for the T- and B-cell responses. LF has been found to increase expression of the complement 3 receptor (C3R) and acquisition of surface IgD. LF has been suggested to act on B-cells to allow for their subsequent interaction with T cells to elevate the antibody response. Structural changes in the N-terminal basic region of LF as well as the basic characteristics of the entire molecule contribute to its interaction with B lymphocytes. Oral administration of LF increases the secretion of IgA and IgG in murine mucosa with intestinal secretion. The effect of LF on T-cell populations can be further delineated in terms of the cellular subset specifically targeted. The adaptive immune response is dominated by T-cell activity, which includes various functions. T-helper cell type 1 (Th1) and type 2 (Th2) stimulate and activate Mf, resulting in intracellular killing events that eliminate intracellular pathogens. LF appears to promote Th1 responses while inhibiting Th2 responses. For example, endogenous LF appears to downregulate allergic rhinitis by upregulating the expression of Th2, Th17 and regulatory T cells. Endogenous LF was suggested to cause T-cell receptor cross-linking (leading to inhibition of T-cell activation), and to cause a reduction in inflammatory factors such as IL-5 and IL-17, further alleviating the degree of inflammation in a murine model of allergic rhinitis (Wang, et al., 2013, Scand. J. Immunol. 78:507-515).

LF accelerates T-cell maturation by inducing the expression of CD4 surface markers through the activation of a transduction pathway. The expression of LF receptors has been reported in all T-cell subsets. Bovine and human LF are capable of binding to surface receptors on the human T-cell line (Jurkat). These associated changes to the surface of molecules that regulate T-cell function suggest that LF is capable of modulating T cell and NK cell activity due to T-cell proliferation. Indeed, LF can potentiate the restoration of the humoral immune response of the host, suggesting a possible mechanism for cell reconstitution through proliferative pathways. LF induces Th1 polarization in diseases in which the ability to control infection or tumor relies on a strong immune response; however, LF may also reduce Th1 cytokines to prevent excessive inflammatory responses (Siqueiros-Cendon, et al., 2014, Acta Pharmacol. Sin. 35(5):557-66).

As noted above, Neolactoferrin, a combination of recombinant human lactoferrin (90%) and goat lactoferrin (10%) isolated from the milk of transgenic goats carrying the full-length human lactoferrin gene, was found to enhance production of IL-1β in vitro. Specifically, iron-saturated Neolactoferrin was reported to increase synthesis of pro-inflammatory cytokine TNFα, which then determined the direction of the differentiation of precursor dendrite cells. Under the action of T cells, Neolactoferrin was reported to amplify the expression of some transcription factors responsible for the differentiation of Th- and Treg-cells and to stimulate the production of both IFNγ and IL-4. These researchers reported that Neolactoferrin exhibits an immunotropic activity and hinders the development of immune inflammatory processes. In contrast to the present disclosure using lactoferrin compositions, the pro-inflammatory activity of Neolactoferrin was dependent on the state of iron saturation, and no significant effect was observed on the expression of “pro-inflammatory” gene TBX21 (encoding the Tbet factor of Th1 cells) or RORC (encoding the RORc factor of Th17 cells) (Chernousov, et al., 2013, Acta Naturae. 5(4):71-77).

However, those studies specifically report no effect on TBX21 and RORC, contrary to the present disclosure. Furthermore, saturating the Neolactoferrin with iron abolishes the activity and actually promotes pro-inflammatory events. Thus, and without being bound by theory, it is believed that Neolactoferrin is a distinct substance from the presently claimed LF. In the presently described compositions and methods, both promotion of Treg as well as an inhibition of Th17 cell function (via reduced IFN-y and IL-17) are observed. It appears that Neolactoferrin may require iron in the Chernousov studies because apoLF was the form harvested from the goat. Whatever the difference, the presently described lactoferrin composition does not include Chernousov's Neolactoferrin. The present disclosure provides a novel method using a lactoferrin composition to skew naïve T cells toward a Treg phenotype and to inhibit the Th1/Th17 T cell phenotype and function for treatment of autoimmune diseases and disorders.

The present disclosure meets a long-felt need for an alternative treatment strategy for automimmune diseases and disorders to allow this group of affected individuals to improve their quality of life. To provide these benefits, a therapy must correct the source of inflammation and not just alleviate the consequences of this aberrant immune activation. Accordingly, systemic introduction of lactoferrin should be beneficial for patients.

### IV. EXAMPLES

The following examples are illustrative in nature and are in no way intended to be limiting.

It is documented herein that lactoferrin skews T-cells to the Treg phenotype. In the present disclosure, rhLF has been shown to be a potent modulator of IL-10, comparable to lactoferrin purified from bovine or human sources. rhLF can activate MAPK signaling in a manner similar to human lactoferrin. MAPK inhibition (most notably MEK inhibiton) results in shut-down of rhLF-induced IL-10 and IL-2. Furthermore, various routes of administration of rhLF results in Treg homing to the gut and its associated lymph tissues in healthy mice.

### Example 1

### Anti-Inflammatory Activity of LF in TNF-Driven Murine Ileitis Model

Crohns disease (CD) is believed to develop through a dysregulated mucosal immune response toward the commensal enteric flora in genetically susceptible individuals. Multiple animal studies indicate that regulatory T cells (Treg) regulate the immune response in normal intestinal mucosa and thereby prevent colitis development. A breakdown of this tolerance to luminal antigens plays a role in IBD development. The plant produced human milk protein described herein can modulate the adaptive immune system in Crohn's disease to ameliorate inflammation.

In some embodiments, a TNF-driven model of CD was used. The murine TNP^{ΔARE} model has a genetic deletion of 69 bp within the AU-rich element (ARE) of the TNF gene in mice (i.e. TNF^{ΔARE}), which confers increased TNF mRNA stability leading to systemic TNF overproduction and development of chronic inflammation localized to the terminal ileum, reminiscent of human colitis in its histological features and the pivotal role played by TNF in its pathogenesis (Kontoyiannis, et al., 1999, Immunity 10:387-98). The gut-specific manifestations of TNF^{ΔARE} mice consist of mucosal abnormalities with intestinal villous blunting, architectural distortion, with associated mucosal and submucosal infiltration of chronic as well as acute inflammatory cells. As disease chronicity continues, the inflammatory infiltrate extends deep into the muscular layers of the bowel wall, with characteristics typical of transmural inflammation (Kontoyiannis, et al., 1999, Immunity 10:387-98).

The presently described plant produced human milk protein was provided by oral gavage or by subcutaneous Alzet pump to 10-14 week old TNF^{ΔARE} mice with ileitis. Specifically, recombinant human LF diluted in PBS (500 mg/kg/day or 50 mg/kg/day; 200 μl bolus) was administered to 12-14 week old TNF^{ΔARE} mice continually over a 14 day period. As a positive control, anti-TNF monoclonal antibody (5 mg/kg) was administered intraperitoneally, twice weekly. After 2 weeks these mice were evaluated for intestinal permeability by FITC dextran flux, inflammation by histology, cell isolation and flow cytometry as well as Treg function. Finally, IL-10 and IL17 output was measured both by ELISA and by intracellular cytokine staining. Upon termination of the experiment, mice were sacrificed and tissues collected for further analysis by histology, T cell profiling and cytokine secretion.

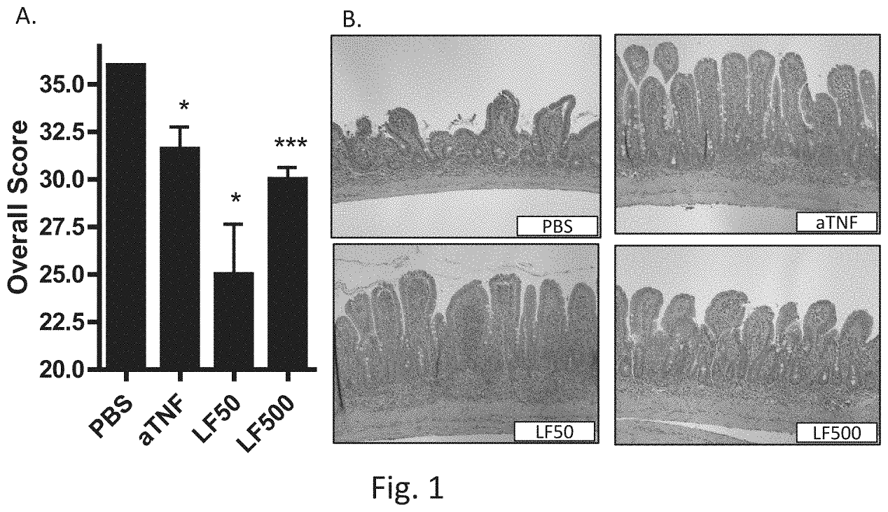

FIG. 1 demonstrates a significant protection of the intestine with both low and high dose administration of LF (p>0.05 and p>0.001, respectively, student's t-test). As shown in FIG. 1, terminal sections of the ilea were treated with PBS alone, anti-TNF (“aTNF”), 50 mg/kg LF (“LF50”), or 500 mg/kg LF (LF500). Sections were fixed in paraformaldehyde and mounted onto paraffin blocks for H&E staining. Scoring was carried out in a blinded manner by a trained pathologist on parameters of active and chronic inflammation, and villous architecture. (*p<0.05, ***p<0.0001, Student's T test. FIG. 1B shows representative H&E images from each treatment group. Most notably was the preservation of normal tissue architecture, with a clear restoration of villus height and the presence of mucus secreting goblet cells (unstained cells embedded within the mucosal epithelium, FIG. 1).

rhLF modulates cytokine expression in ileal explants. In order to assess molecular readouts of protection, a small section of the terminal ileum (˜5 mm²) was isolated and placed in culture over a 24 hour period in RPMI medium 10% FBS+100U Penicillin/100 μg/ml streptomycin. Culture supernatants were then collected and secretion of cytokines was assessed by ELISA (Mouse TNF, Mouse IL-10, Mouse IL-17a ELISAs Ready-Set-Go!™, e Biosciences). Upon termination of experiment, terminal ilea were collected from each mouse (n=4 for each treatment group) and cultured in RPMI+10% FBS+100U Penicillin/100 ug/ml streptomycin for 24 hrs.

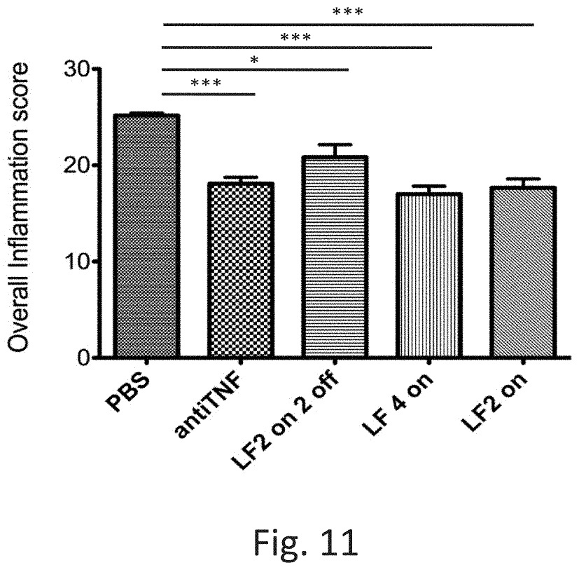

Isolated CD4⁺ CD25⁻ T cells exhibited decreased proliferation in the presence of plant produced human milk protein. These cells produced increased quantities of IL-10. In the TNF^{ΔARE} mouse model of IBD after 2 weeks of treatment with the plant produced human milk protein versus vehicle there was a significant drop in influx of Naive CD44lowCD62Lhi T cells into the lamina propria of the intestine. In addition to this there was a significant increase in Treg numbers and these cells produced increased quantities of IL-10. Histology of the intestines from these mice was evaluated by a pathologist in blinded fashion and all indices of inflammation were improved (see Example 7 and FIG. 11). Similar studies were carried out in the dextran sodium sulfate induced colitis “DSS” model, and similar anti-inflammatory effects were noted (see Example 11, below).

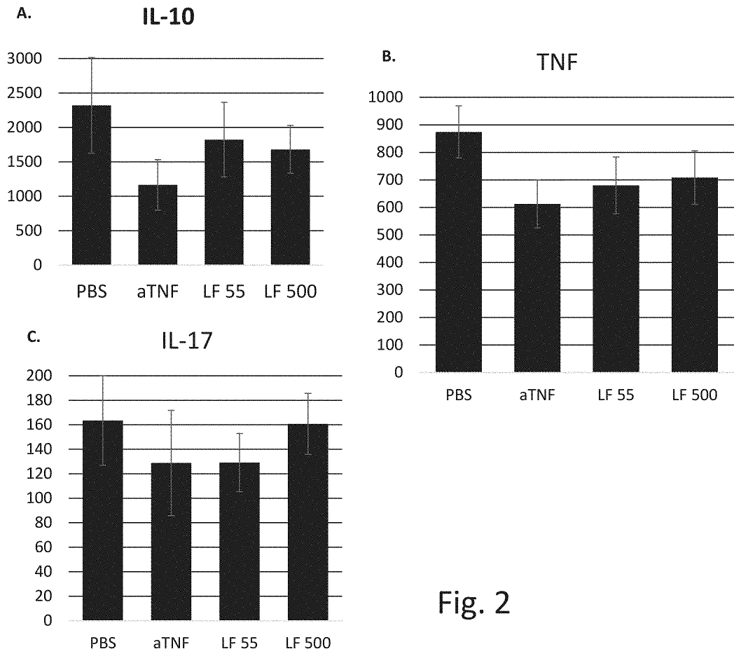

As shown in FIG. 2, culture supernatant was then collected and assessed for levels of IL-10 (FIG. 2A), TNF (FIG. 2B), and IL-17 (FIG. 2C) in separate ELISA assays. IL-10 was elevated in PBS-treated mice indicating manifestation of inflammatory disease that is reduced by anti-TNF and rhLF. As expected, high levels of TNF were detected in PBS-treated mice that are reduced by anti-TNF and rhLF. This LF reduction of proinflammatory cytokine secretion was mirrored by mRNA reduction in whole ileal tissue in TNF^{ΔARE} mice.

rhLF administration significantly decreased naive T cell infiltration and proliferation in the intestinal lamina propria, mesenteric lymph nodes and spleen of 12 weeks old TNF^{ΔARE} mice. An increase in CD4+Foxp3+ regulatory T cells was noted in the rhLF-treated mice. These rhLF treated mice also exhibited improved histological indices and had improved intestinal barrier function, indicating efficacy for rhLF to decrease inflammation in a preclinical model of CD.

Thus, it was effectively demonstrated that the plant produced human milk protein has therapeutic potential in murine models of inflammatory bowel disease. Overall, for all three cytokines evaluated, a trend towards decreased cytokine secretion from ex vivo ileal tissue was observed (FIG. 2, A-C), indicating an overall decrease in inflammation and subsequent decrease in cytokine output at the source of inflammation. However, given the plethora of cell types present within the whole ileal tissues sampled (leukocytes, epithelia stromal fibroblasts, myofibroblasts, pericytes, endothelial cells and smooth muscle cells) (Koning J. J. and Mebius R. E. 2012, Trends Immunol. 33:264-70; Pinchuk, et al., 2010, Curr. Gastroenterol. Rep. 12:310-8; Powell, et al., 2011, Annu. Rev. Physiol., 73:213-37), the role CD4⁺ T cells may be playing in the resolution of inflammation in response to LF administration was uncertain.

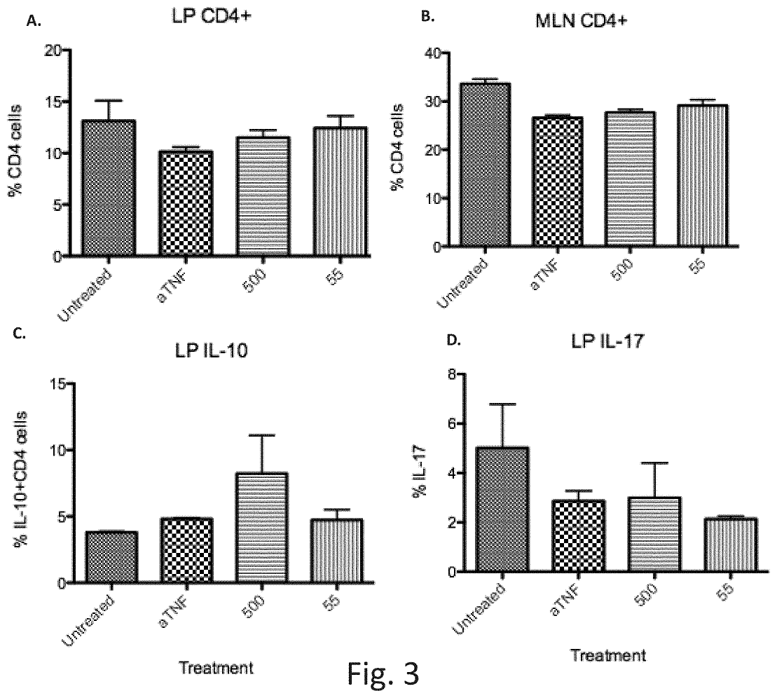

rhLF enhanced levels of FoxP3 mRNA in whole ileal tissue, and decreased CD4+ lymphocyte burden in the lamina propria and the draining mesenteric lymph nodes in TNF^{ΔARE} mice. To gain further insights into the mechanism, CD4⁺ T cells were isolated from the lamina propria (LP) and mesenteric lymph nodes (MLN) and assessed for phenotype by flow cytometry. By looking at the burden of all CD4⁺ T cell subtypes, it was demonstrated that LF reduced cellularity both locally at the site of inflammation (LP, lamina propria; FIG. 3A), and at the draining lymph nodes (MLN, mesenteric lymph nodes; FIG. 3B). This indicated an overall decrease in inflammatory activity in response to LF administration. In an effort to characterize the inflammatory profile of T cells at the site of inflammation, tissues were stained for the cytokine markers of Treg and Th17 cells, IL-10 and IL-17, respectively. Here, it was demonstrated that LF significantly induced IL-10 expression with a concomitant decrease in IL-17 in LP CD4⁺ cells (FIGS. 3C and D, respectively).

rhLF modulates FoxP3 mRNA levels in whole ileal sections and reduces T Cell infiltration. Upon termination of experiment, ileal sections (c. 5 cm terminal) and snap frozen. Tissues were homogenized in 300u1 buffer RLT using an Omni Tissue Homgenizer (3 rounds of 10s). RNA was then extracted using RNEasy kit (Qiagen) and quantified at OD₂₆₀ (Nanodrop). Equal amounts (100 ng) of RNA were reverse transcribed with high capacity cDNA reverse transcription lit (ABI). FoxP3 mRNA was amplified by qPCR using taqman primers (ABI), with 18S as an internal control for each well. Fold changes were determined using the 2ΔΔCT Method. Quantified mRNA levels mirror cytokine secretion obtained by cultured explants. FoxP3 mRNA levels were found to be increased in rhLF-treated mice. At sacrifice, spleens were removed and mashed through a 70 um filter. Total cells were counted using trypan blue exclusion. Both anti-TNF and high dose of rhLF induced a reduction of total cellularity in the spleen. Lymph nodes (MLN) and the lamina propria (LP) were removed and digested with collagenase and subsequently homogenized. T cells were removed via negative selection and CD4+ cells were quantified immediately. CD4+ cell infiltration was found to be reduced in both the anti-TNF-treated and rhLF treated animals versus the PBS control in both the lamina propria (LP) (See FIG. 3A), and the mesenteric lymph nodes (MLN) (FIG. 3B). T cells were isolated from the ileal lamina propria (LP) and mesenteric lymph nodes, CD4⁺ T cells were isolated by negative selection and CD4⁺ positive cells were determined by flow cytometric analysis.

Ileal lamina propria (LP) CD4⁺ T lymphocytes were isolated using negative selection and stimulated in the presence of PMA, Ionomoycin, Brefeldin A for 4h. Cells were stained with anti-CD4, anti-IL-10 and anti-IL-17. Flow cytometric analysis demonstrated that at the higher dose, LF induced expression of IL-10 (See FIG. 3C) and decreased the expression of IL-17 in the CD4⁺ T cells present within the local area of inflammation, namely the LP (FIG. 3D) at both high and low dose. Thus, lactoferrin is able to resolve inflammation through the induction of expression of the anti-inflammatory cytokine IL-10, whilst reducing the expression of the pro-inflammatory IL-17. This is suggestive of a shift in balance from a Th17 to a Treg phenotype.

Together, these data demonstrate that in a mouse model of IBD, oral administration of LF decreases the severity of TNF-driven disease as evident by a protection of tissue at the histopathological level. Further investigations show that LF reduces cytokine secretion in intestinal explants, and that overall inflammation is decreased as evident by a decrease in CD4⁺ cellularity at the LP and MLN.

### Example 2

### Lactoferrin Induces a Pro-Regulatory T Cell Phenotype

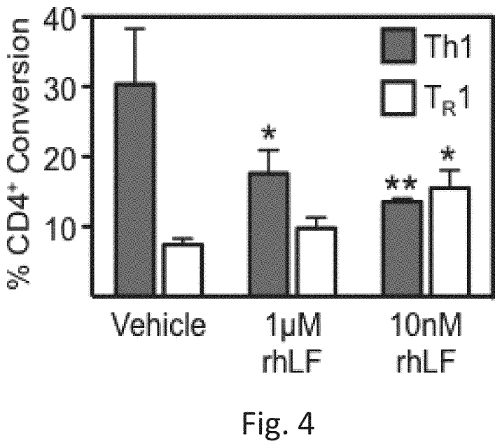

Conversion of CD4⁺CD25⁻ naïve T cells under polarizing conditions is skewed by LF to a pro-regulatory phenotype. In order to investigate the underlying mechanisms responsible for the protection of TNF^{ΔARE} mice against severe inflammation, the ability of LF to promote a pro-regulatory phenotype in cultured T cells was tested. For this assay, murine naïve CD4⁺CD25⁻ T cells were isolated and placed into conversion culture for three days. Conversion culture contains either: 1) TGFβ, IL-2 and plate-bound anti-CD3 to activate conversion into T_{R}1 cells (CD4⁺FoxP3⁺, IL-10 secreting regulatory T cell) or 2) plate-bound anti-CD3, anti-CD28, IL-2, anti-IL-4, and IL12, to activate conversion of the naïve CD4⁺ CD25⁻ T cells into interferon-γ (IFNγ) secreting, proinflammatory Th1 cells. Cells were then restimulated with PMA/Ionomycin/Brefeldin A and assayed by intracellular cytokine staining to identify IFNγ and IL-10⁺ cells. Results represent mean±SEM for n=4 individual wells. *p<0.05, **p<0.01. LF was found to significantly induce an increase in T_{R}1 cell conversion while at the same time decreasing the conversion of naïve CD4⁺ cells into Th1 cells (FIG. 4). The expression of the anti-inflammatory cytokine IL-10 by all CD4⁺ T cells and especially Treg cells was increased in in vitro stimulated cells. Expression of IL-10 was more dramatically upregulated in Treg cells, by as much as 3-fold.

Together, these data demonstrate for the first time that LF acts directly to modulate the immune response in a TNF-driven model of Crohn's like ileitis, with rescue of normal intestinal physiology as demonstrated by enhanced gut barrier function. Furthermore, LF reverses the severe chronic-stage pathology seen in both TNF^{ΔARE} and DSS mice, with reversal of tissue damage and a decrease in associated T cell infiltration. LF was also demonstrated to have a role in decreasing inflammation in a DSS model of colitis through skewing the phenotype of CD4⁺ cells away from a proinflammatory Th1/17 phenotype, towards a regulatory Treg phenotype.

In a pilot study to examine the mechanisms which underpin the pro-resolution role of LF, the ability of LF to promote conversion of T_{R}1 cells with a concomitant decrease in Th1 population of cells was demonstrated. Furthermore, increased IL-10 output was demonstrated in total CD4⁺ T cells, and to an even greater extent in regulatory CD4⁺ Foxp3⁺ Treg cells. These data will be the basis of further investigation of the mechanism of action of orally administered LF to reduce inflammation in both the TNF^{ΔARE} model, alongside a T cell driven adoptive transfer model, which together have been demonstrated to be excellent predictors of efficacy in the context of human disease (Valatas, et al., 2013, Am. J. Physiol. Gastrointest. Liver Physiol., 305:G763-85; DeVoss J. and Diehl L., 2014, Toxicol. Pathol. 42:99-110; Koboziev, et al., 2011, Inflamm. Bowel Dis., 17:1229-45). Future studies will yeild molecular targets of LF that modulate intracelluar signaling to impair Th17 function and promote Treg phenotype

### Example 3

### LF Shifts Gene Expression Away from a Th17 Towards a Treg Profile in Primary CD4+ T Cells

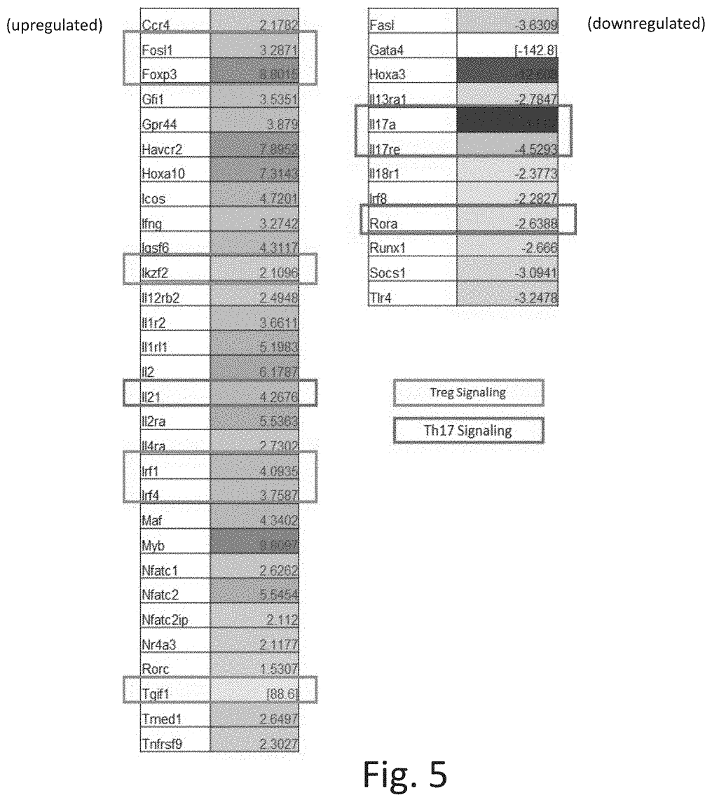

In an attempt to analyze numerous genes involved in T cell differentiations, a commercial PCR array screen with specified targets implicated in driving specific lineages of T cells (T Helper Cell Differentiation PCR Array, SA Biosciences) was employed. CD4⁺ T cells were isolated from spleens and mesenteric lymph nodes of healthy C57/BL6 mice and purified by negative selection (CD4⁺ T Cell Isolation Kit, mouse, Miltenyi Biotec), and stimulated (T Cell Activation/Expansion Kit, Miltenyi Biotec) for 24h in the presence or absence of 1 μM LF. RNA was subsequently harvested (RNeasy, Qiagen) and cDNA prepared (High-Capacity cDNA Reverse Transcription Kit, Applied Biosystems) and qPCR performed using the primers supplied with the T Helper Cell Differentiation PCR Array. Threshold cycles (CT) were determined for the gene of interest and a housekeeping gene (β-Actin) and fold-change of LF stimulated over unstimulated determined using the 2ΔΔCT method. Select targets (fold change±2) are shown in the FIG. 5. LF appears to positively regulate a number of genes involved in Treg phenotype, with concomitant decrease in Th17 signals. Here, LF treatment was found to upregulate a number of genes which are known for their involvement in Treg generation (Fosl1, Foxp3, Ikzf2, Irf1, Irf4, Tgif), with a concomitant downregulation of canonical regulators of Th17 phenotype (Il-17a, Il17re, Rora). Together, these data strongly indicate the ability for LF to drive specific gene regulation in CD4⁺ T cells in a manner which drives their phenotype away from Th17 towards a Treg fate.

### Example 4

### Lactoferrin Treatment Enhances the Treg-Expanding Factor IL-2

The importance of IL-2 to regulation of T cells was initially identified by the requirement for IL-2 receptors for the generation and expansion of Treg (Malek, et al., 2002, Immunity, 17:167-78; Almeida, et al. 2002, J. Immunol. 169:4850-60). The role that LF may play in the regulation of T cells through its ability to upregulate IL-2 in vitro was then investigated. A preliminary qPCR screen identified IL-2 mRNA as being upregulated upon T cell stimulation with LF (FIG. 5, right panel). To further validate this, murine primary cells were screened for upregulation of IL-2 over a 24 hour time period. To achieve this, CD4⁺ T cells were isolated from spleens of healthy C57/BL6 mice, homogenized, red cells lysed, and enriched for CD4⁺ cells by negative selection using magnetic sorting. Proliferating T cells were stimulated by plate bound antiCD3/CD28 in the presence or absence of 1 μM rhLF over 2, 4, 6, 18 and 24h time periods, andRNA was purified from lysates using RNEasy extraction kit (Qiagen). Equal amounts (100 ng) of RNA were reverse transcribed with high capacity cDNA reverse transcription kit (ABI). qPCR was carried out using SA Biosciences T cell differentiation array. A heatmap was generated for upregulated (light-dark green, shown as light to dark gray on the left side of FIG. 5) and downregulated genes (yellow-red, shown as light to dark gray on the right side of FIG. 5). IL-2 target was amplified by qPCR using Taqman primers (ABI), with 18S as an internal control for each well. Fold changes were determined using the 2ΔΔCT. The induction of IL-2 was evident as early as 6h post treatment, and sustained as far as 24h.

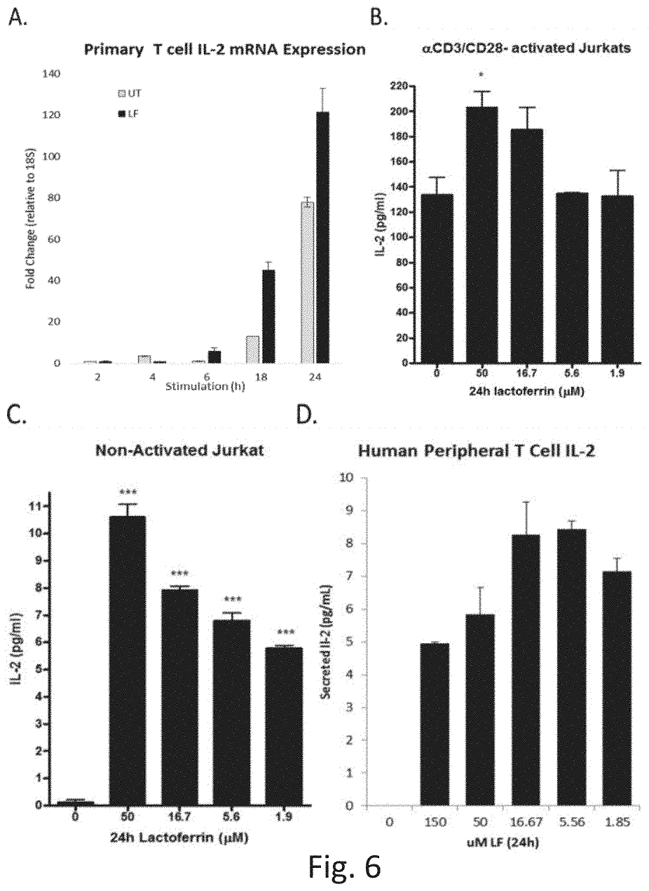

LF drives the expression and secretion of IL-2 in T cells. Primary T cells were isolated from spleens of healthy C57/BL6 mice and enriched for CD4+ cells by negative selection. Cell proliferation was induced by plate bound antiCD3/CD28 in the presence or absence of 1 uM rhLF over 2, 4, 6, 18 and 24 hour time periods. qPCR analysis demonstrated that LF enhances IL-2 gene expression in activated primary murine CD4⁺ T cells as early as 6h, and sustained throughout the duration of the experiment until 24h post-treatment (FIG. 6A). To validate this observation in terms of protein secretion, unstimulated and anti CD3/CD28-stimulated cells were incubated in the presence of various doses of LF for 24h and subsequently collected cell supernatant for detection of IL-2 by ELISA (Mouse IL-2 ELISA MAX™ Deluxe, Biolegend). Using the Jurkat immortalized line of human T lymphocyte (ATCC), LF was demonstrated to significantly induce secretion of IL-2 in both stimulated (activated; FIG. 6B) and unstimulated (non-activated; FIG. 6C) cells in a dose-dependent manner. Furthermore, to demonstrate that LF had activity in primary human cells, human peripheral blood T cells (Stem Cell Technologies) were incubated in the presence of LF for 24 hrs. As with the immortalized Jurkat line, LF was found to induce IL-2 secretion in non-activated primary human T cells over a 24 hr period from an undetectable baseline to ˜10 pg/ml (FIG. 6D). Together, these data demonstrate the ability for LF to induce IL-2 expression in a time- and dose-dependent manner, and suggests the underlying mechanism by which LF drives the generation and expansion of CD4⁺ CD25⁺Foxp3⁺ regulatory T cells.

### Example 5

### Lactoferrin Treatment of Human Patient with an Autoimmune Disease

Patients will be treated orally with the lactoferrin compositions of the present disclosure by administering a 1 g capsule or dispersed powder in a buffered solution. Treatments will be daily administration, up to 3 g per day. Patients will be monitored for disease activity indices (such as Crohn's disease activity indices (CDAI) or ulcerative colitis activity indices (UCAI)). Patients will also answer questions related to quality of life (QoL) using questionnaires that have been tailored and tested for each autoimmune disease. Blood biomarkers may also be used to assess the effectiveness of treatment with the lactoferrin compositions, such as the inflammatory marker C-reactive protein (CRP), or the levels of inflammatory cytokines, TNF, IL-6, IL-12, IL-lb. Specific organ biomarkers will be assessed, such as the marker of intestinal inflammation, calprotectin which can be assessed in stool samples from IBD patients.

### Example 6

### CD8+ T Cells are Also a Target for LF Modulation Towards a Treg Profile

It is now widely accepted that type 1 diabetes (T1D) is an autoimmune disease associated with the activation of CD4 and CD8 T cells recognizing islet autoantigens. In the context of a T cell-mediated autoimmune disease such as type 1 diabetes, CD4 and CD8 T cell recognition of islet autoantigenic epitopes is a key step in the autoimmune cascade. Both CD4+ Th17-cells and CD8+ cytotoxic T lymphocytes (CTLs) are believed to be involved in both T1D and experimental autoimmune encephalomyelitis (EAE). T1D is caused by autoimmune destruction of insulin-producing islet β cells of the pancreas. Antigen-specific CD8⁺ T cells are found in the peripheral blood of T1D patients. Studies in a nonobese diabetic (NOD) mouse model of T1D have indicated that CD8⁺ T cells inflict damage to islet β cells both at the early stage in diabetes development and at the final effector phase of the disease. There is also some preliminary evidence showing that Th17 cells may be considered as a contributing factor in the pathogenic process of T1D. For example, it has been found that IL-17 is expressed in the pancreas of T1D mouse model, and the reduction of Th17 cells with the induction of IFN-γ inhibited IL-17 production and restored normoglycemia at the prediabetic stage. CD8 T cell epitopes for human β cell antigens include glutamic acid decarboxylase (GAD65), insulinoma-associated protein 2 (IA-2), islet amyloid polypeptide (IAPP) (prepro), islet-specific glucose-6-phosphatase catalytic subunit-related protein (IGRP) and insulin; CD8 T cell epitopes for mouse β cell antigens include dystrophia myotonica kinase (DMK), GAD65, GAD67, IGRP, insulin 1/2 and insulin 2. In contrast with CD4, for CD8 T cells the major contributor to epitopes recognized in the mouse is IGRP, and in man is proinsulin/insulin (Lorenzo, et al., 2007, 148(1):1-16).

Because there is increasing evidence that CD8⁺FoxP3⁺ Treg cells are mediators of autoimmune disease, it was hypothesized that Th cells share a lactoferrin receptor that is expressed regardless of their CD4/CD8 profile, and LF may drive FoxP3 expression in both lineages. The lactoferrin composition disclosed herein can be tested in experiments similar to Example 3, supra, for its ability to drive FoxP3 expression in CD8⁺ T cells.

The EasySep™ Mouse CD8+ T Cell Isolation Kit or the RoboSep™ Mouse CD8+ T Cell Isolation Kit (Stemcell Technologies, catalog #19853 and #19853RF, respectively) are designed to isolate CD8+ T cells from single cell suspensions of splenocytes or other tissues by negative selection. Unwanted cells are targeted for removal with biotinylated antibodies directed against non-CD8+ T cells (CD4, CD11b, CD11c, CD19, CD24, CD45R/B220, CD49b, TCRγ/δ, TER119) and streptavidin-coated magnetic particles (RapidSpheres™). Labeled cells are separated using an EasySep™ magnet without the use of columns. Desired cells are poured off into a new tube.

CD8+ T cells are stimulated for 24h in the presence or absence of 1 μM LF. RNA is subsequently harvested (RNeasy, Qiagen) and cDNA prepared (High-Capacity cDNA Reverse Transcription Kit, Applied Biosystems) and qPCR performed using the primers supplied with the T Helper Cell Differentiation PCR Array. Threshold cycles (CT) are determined for the gene of interest and a housekeeping gene (β-Actin) and the fold-change of LF stimulated over unstimulated determined using the 2ΔΔCT method. It is predicted that LF will be found to upregulate specific genes responsible for the generation of Treg and or downregulate genes responsible for the generation of IL-17 in CD8⁺ T cells.

Taken together, these data strongly indicate that LF has protective effects in autoimmune pathology, as demonstrated with the TNF^{ΔARE} model of spontaneous Crohn's-like ileitis. Administration of LF was found to significantly reduce disease in this model, similar to the induction of remission in IBD. Further analyses demonstrated that the administration of LF reduced inflammatory burden through decreased cytokine secretion profile. This was accompanied by the observation that LF tempered the overactive autoimmune response through promotion of IL-10 producing Treg at the expense of IL-17 producing Th17 cells, both in vitro and in vivo. Mechanistic analyses demonstrated that the action of LF was through the regulation of gene transcription whereby genes responsible for the generation of Treg were upregulated, with the concomitant down-regulation of genes responsible for the generation of IL-17. Furthermore, when investigating the regulation of IL-2, an important factor for the development and expansion of Treg, LF was shown to significantly induce gene and protein expression in a time- and dose-dependent manner in murine and human T cells.

Therapeutic or prophylactic administration of the presently claimed lactoferrin compositions to a subject in need of treatment of an autoimmune disease or disorder can upregulate a number of genes involved in Treg generation (Fosl1, Foxp3, Ikzf2, Irf1, Irf4, Tgif), with a concomitant downregulation of canonical regulators of the Th17 phenotype (Il-17a, Il17re, Rora), and is a promising approach for treatment of autoimmune diseases and disorders.

### Example 7

### Continual Administration of rhLF Over 4 Wks. Vs. (2 Wks. on, 2 Wks. Off) Vs. (2 Wks. Off, 2 Wks. On) in TNF^{ΔARE} Mice

To address the duration of rhLF administration (at 500 mg/kg/day), an experiment was devised where TNF^{ΔARE} mice were treated for either 4 weeks continuously with rhLF (“LF 4 on”), for 2 weeks rhLF followed by 2 weeks washout with PBS (“LF 2 on 2 off”), or for 2 weeks with PBS followed by 2 weeks rhLF (“LF 2 on”), and with anti-TNF monoclonal antibody as a positive control, 5 mg/kg, twice per week. At the end of the 4 week experiment, mice were sacrificed and ilea were prepared for histology, as described previously. A significant decrease in inflammatory parameters associated with iletis was demonstrated in all groups treated with rhLF (FIG. 11), as assessed by a pathologist in a blinded manner. Furthermore, the withdrawal of rhLF and subsequent washout resulted in a slightly reduced protective effect, suggesting the possibility of the reemergence of inflammation upon withdrawal of treatment.

### Example 8

### rhLF is Effective at Inducing IL-10 Secretion in Freshly Isolated CD3+ Lymphocytes and LF Derived from Bovine Colostrum (bLF) or Isolated from Human Milk (hLF) has Similar Activity, but at Lower Potency

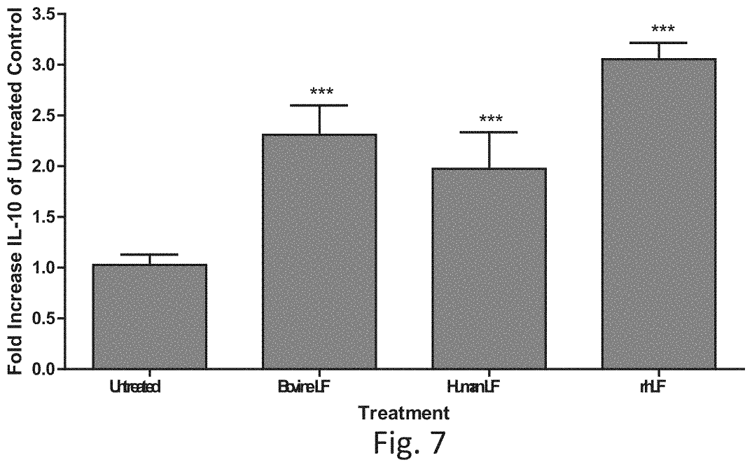

Human Blood was incubated with an antibody cocktail to negatively select for CD3⁺ cells using immunodensity separation (RosetteSep™ Human T cell Enrichment Cocktail, Stem Cell Technologies). Blood was then overlayed on a ficoll density gradient (Lymphoprep™, Stem Cell Technologies) and centrifuged at 1200×g for 30 minutes with the brake off. The buffy coat, consisting of purified CD3⁺ cells was then collected, and pelleted. Red blood cell lysis was performed and the pellet then washed 2× in dPBS+2% FBS. A final wash with dPBS was carried out to remove any crossover FBS. Cells were then plated at a density of 1e6/ml in XVIVO-15™ medium (Lonza), supplemented with 5 ng/mL IL-2 (Peprotech™). Cells were treated with 10 uM LFs isolated from human milk, bovine colostrum (Sigma Aldrich), or the rhLF described herein. After 72h treatment, supernatants were collected and IL-10 production assessed by ELISA (Human IL-10 ELISA MAX™, eBioscience). FIG. 7 shows that treatment of CD3⁺ cells with all LFs tested resulted in a 2-3 fold increase in IL-10 output (p<0.001). Interestingly, the greatest induction of IL-10 was seen following treatment of cells with rhLF, followed by bovine colostral LF and LF from human milk. Thus, rhLF induces secretion of IL-10 more effectively than LF isolated from bovine colostrum (Bovine LF) or from human milk (Human LF) in human CD3⁺ lymphocytes over a 72 hour treatment period. This is the first time that the direct activity of various forms of LF has been demonstrated to induce lymphocyte secretion of the potent anti-inflammatory cytokine, IL-10.

### Example 9

### rhLF Acts Trough MAPK Pathways to Induce a Cytokine Response in Primary and Jurkat Human T Cell Lines

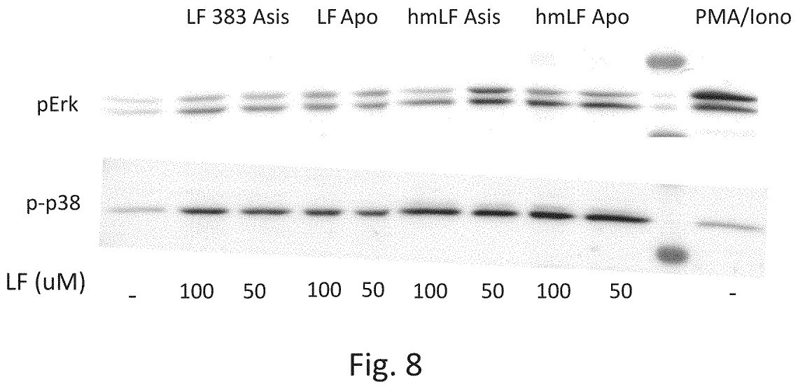

To interrogate the molecular events upstream of LF induced IL-10 activation, the p44/42 extracellular signal-regulated kinases (Erk) and p38 branches of the mitogen activated protein kinase (MAPK) pathway were tested. For this, CD3⁺ cells were isolated as described above, and treated with either 100 uM or 50 uM LF for 72h and Western blotting analysis carried out for phopshoylated Erk (pErk) or phosphorylated p38 (p-p38). As a positive control, cells were activated with PMA/Ionomycin (20 ng/mL, and 1 ug/mL, respectively) for 10 minutes prior to subsequent harvest. The isoforms of LF used were partially iron saturated rhLF (LF 383 Asis), iron-depleted rhLF (LFApo), partially iron-saturated LF from human milk (hmLF Asis, Athens Research and Technology), or iron-depleted LF from human milk (hmLF Apo, Athens Research and Technology). After 72h stimulation with the various forms of LF, the cells were lysed in 2× Laemmle's sample buffer (Sigma Aldrich), and denatured and reduced by boiling at 95 C for 5 min in the presence of 5 mM tris(2-carboxyethyl)phosphine (TCEP; Thermo Fisher). 30 uL of each sample was electrophoresed using a Novex™ 4-20% Tris-Glycine Mini Protein Gels (Life Technologies), and subsequently transferred to a nitrocellulose membrane. The membranes were blocked using 3% non-fat dry milk/TBS-T 0.1% for 1h at room temperature. Membranes were then probed with Phospho-p44/42 MAPK (Erk1/2) (Thr202/Tyr204) or Phospho-p38 MAPK (Thr180/Tyr182) antibodies (#9101, #9211, respectively; Cell Signaling Technologies) overnight at 4 C. After subsequent washing in TBS-T0.1%, membranes were probed with Anti-rabbit IgG, AP-linked Antibody and developed 1-Step™ NBT/BCIP Substrate Solution (Life Technologies). FIG. 8 demonstrates the ability for all forms of LF to induce phosphorylation of Erk and p38, albeit with some variation in the magnitude of activation. In particular, rhLF induces phosphorylation of the p38 and Erk signaling cassettes of the MAPK cascade.

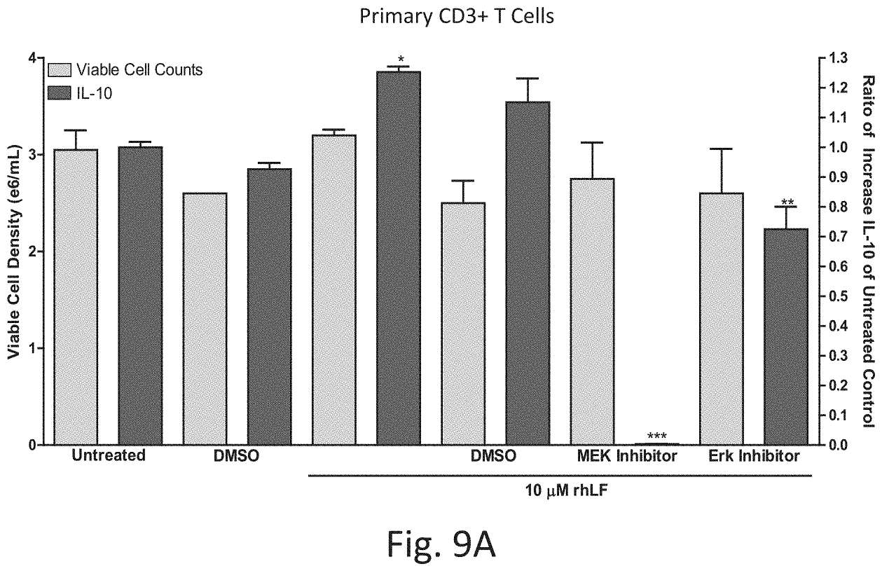

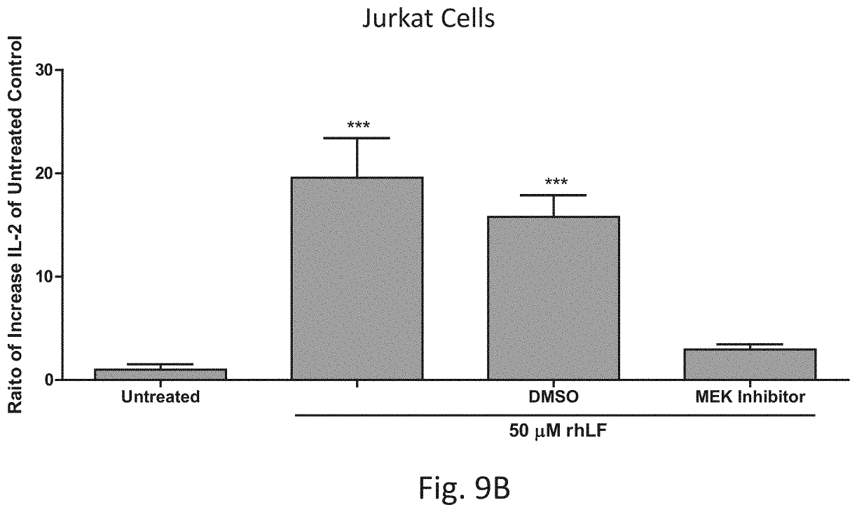

To test the role for Erk signaling pathway as being an upstream mediator of rhLF induced IL-2 and IL-10 secretion, selective inhibitors of MEK (50 nM, Binimetinib [MEK162], Array Biopharam), or Erk inhibitor (10 uM, ERK Inhibitor II [FR180204], EMD Millipore) were used. IL-10 production in primary CD3⁺ cells was assessed, as detailed previously, however, one hour prior to incubation with rhLF (10 uM), CD3⁺ cells were pretreated with 50 nM of MEK or 10 uM Erk inhibitor. To address the potential toxicity that the use of small molecule inhibitors of MAPK may have on these cells, a sample of the cell suspension was collected at the time of termination of the experiment and cell viability determined. FIG. 9 shows that rhLF acts though the Erk signaling cascade to induce secretion of IL-10 in freshly isolated human CD3+ cells (FIG. 9A) or IL-2 in the Jurkat cell line (FIG. 9B).

FIG. 9A demonstrated that when cells were pretreated for 1 hr with 50 nM MEK162 prior to 72 hrs rhLF (P<0.001); the use of MEK inhibitor resulted in a complete loss of rhLF-induced IL-10 production in CD3⁺ cells (p<0.001, one-way ANOVA), whereas inhibition of Erk resulted in a roughly 30% decrease in IL-10 production (p<0.01, one way ANOVA). Thus, inhibition of Erk with 10 uM FR180204 resulted in a significant (p<0.01) albeit less pronounced inhibition when compared with MEK162. Neither inhibitor had a significantly detrimental effect on the viability of the cells (FIG. 9A light gray bars), suggesting that the decrease in IL-10 production is not a function of reduced cell number, but due to the inhibition of the selected pathways. As MEK is further upstream from Erk in this signaling pathway, it is conceivable that there is an alternative pathway which may be circumventing MEK to lead to some degree of preservation of rhLF-induced IL-10 signaling in this cell line.

To assess the effects of MAPK inhibition of IL-2 in human T cells, the Jurkat cell line was once again used. The reason for this is that primary human CD3⁺ cells require supplementation of 5 ng/mL IL-2, which would mask any effects of rhLF on the production of that cytokine. Jurkat cells were treated as described for the CD3⁺ cells. As shown in FIG. 9B, stimulation of Jurkat cells with 50 uM rhLF resulted in as much as a 20-fold increase over the resting levels of IL-2. However, the use of the MEK inhibitor, MEK162 MEK162 resulted in a roughly 10-fold decrease in rhLF-induced IL-10 and reduction of IL-2 in Jurkat cells to baseline, to levels not statistically significant from basal signaling. Treatment with rhLF (50 uM), or with vehicle (DMSO) resulted in significant increases in IL-2 production when compared with untreated (p>0.001). This increase was reversed in the presence of 50 nM MEK162.

### Example 10

### Oral Delivery of rhLF Directs Treg to Gut and Associated Lymph Organs in Healthy Mice

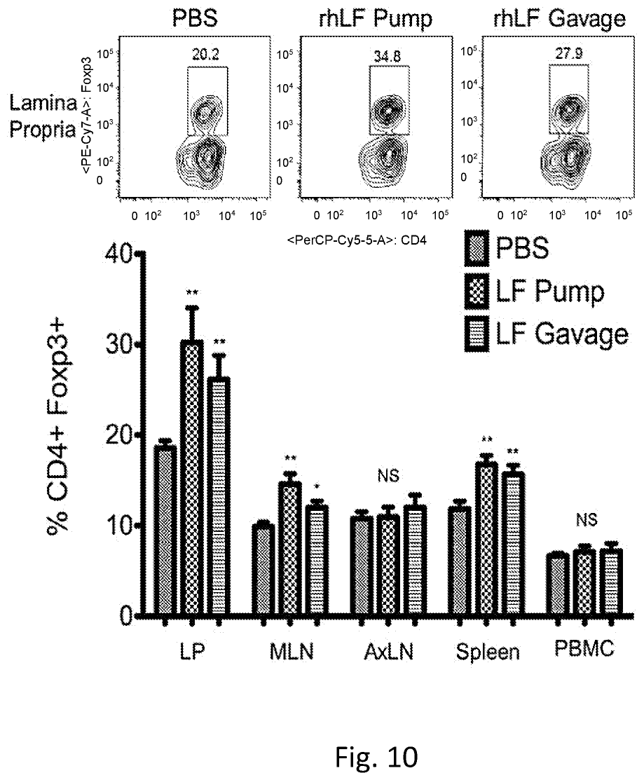

To predict whether rhLF would act as effectively when dosed orally, a 14-day treatment was carried out whereby mice were either treated with sham injection (PBS), rhLF delivered S.Q. by osmatic pump (200 mg/kg/day, Alzet), or by gavage at a dose of 500 mg/kg/day. Upon termination of the experiment, tissues were collected (lamina propria, LP; mesenteric lymph nodes, MLN; axillary lymph nodes, AxLN; peripheral blood mononuclear cells, PBMC; and spleen) and CD4⁺ T cells were isolated by positive selction using magnetic beads (Stem Cell Technologies). Treg populations were subsequently analyzed by flow cytometery based on their expression of the canocial markers CD4 and FOXP3. Healthy, four-week old C56/BL6 mice were treated with rhLF S.Q. (200 mg/kg/day) or gavage (500 mg/kg/day) for a 14 day period. FIG. 10 shows that both subcutaneous (S.Q.) and oral routes of administration of rhLF results in Treg homing to intestinal tissues and associated lymphoid organs. Specifically, flow cytometry revealed that administration of rhLF induces a marked and significant increase in accumulation of Treg populations at the LP, followed by the MLN and then at the spleen. Treg induction did not appear to occur adjacent to the site of insertion of the osmotic pump (AxLN), or within the cirulating PBMC. These data strongly suggest the ability for rhLF to act to direct Treg to the intestinal tissues in the absence of disease, regardless of route of administration. Indeed, it has been reported that Treg homing to the gut is required for immunological tolerance, and that specific homing of Tregs by various factors (Retinoic Acid, Rapamycin) affects their ability to preferentially home to distinct tissues and increase their protective effects a model of T-cell induced murine colitis.

### Example 11

### Anti-Inflammatory Effects of rhLF in a DSS Model of Colitis

To demonstrate that the protective effects of rhLF are not restricted to a single experimental model, and to better cover the broad spectrum of disease phenotypes that IBD encompasses, the dextran sodium sulfate induced colitis (DSS) model of chemically induced colitis was employed. DSS colitis resembles UC in terms of its restricted disease manifestations at the colon and pathological features (Yan, et al., 2009, PLoS One 4:e6073), with overlapping similarities with UC, such as transmural inflammation with disseminated lymphoid follicles (Perse, M. and Cerar, A, 2012, J. Biomed. Biotechnol. 2012:718617). Although hLF and bLF have been demonstrated to reduce inflammation in murine (Haversen et al., 2003, Scand. J. Immunol. 57:2-10) and rat (Togawa, et al., 2002, 1 Gastroenterol. Hepatol. 17:1291-8) models of DSS colitis, until the present study, the effects of recombinant LF on T cell phenotypes had not been investigated. To address this, 12-week old C57/BL6 mice were administered 3% DSS in their drinking water ad libitum for a 5-day period, followed by a 3-day respite period of normal drinking water. Throughout the 8-day course of colitis, mice were administered 500, 250 or 125 mg/kg/day rhLF daily by oral gavage.

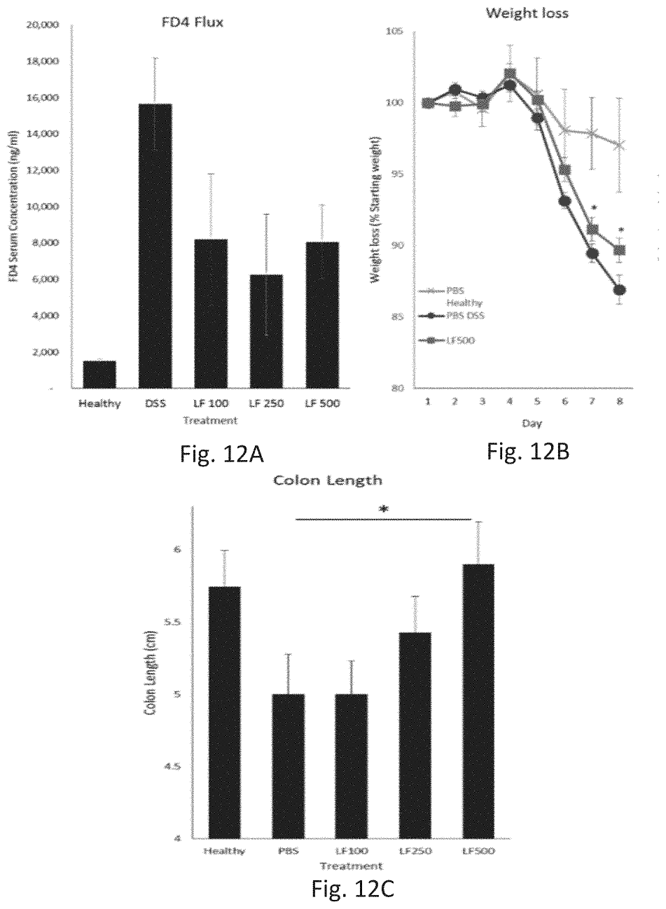

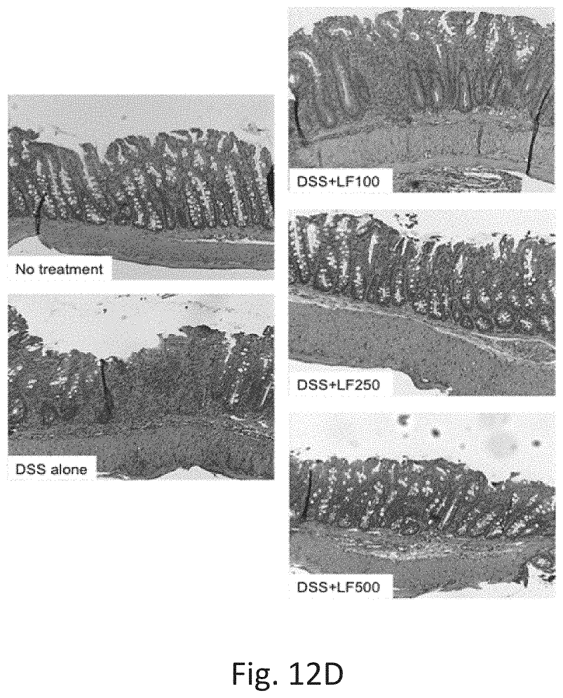

As a readout of disease progression, mice were monitored for weight loss during the course of the experiment. To demonstrate the ability of rhLF to maintain normal gut physiology, intestinal flux movement from the gut to the systemic circulation across the intestinal mucosa of FITC labelled dextran (m.w. 4 kDa) was determined on day 7. Upon examination of epithelial flux, it was observed that mice treated with all doses of rhLF exhibited improved barrier function, as evident through decreased FD4 into the systemic circulation (FIG. 12A).

Furthermore, observation of gross measures of disease demonstrated that the highest dose of rhLF (500 mg/kg) resulted in improved weight loss when compared with sham-treated animals at days 7 and 8 post-DSS treatment (FIG. 12B, p<0.05, two-way ANOVA, with Bonferroni's post-test), with a concomitant preservation of colon length at the highest dose of rhLF (FIG. 12C, *p<0.05, students t-test). H&E staining of the colons from vehicle-treated (DSS alone) mice revealed a complete loss of epithelial integrity and crypt architecture, absence of goblet cells, immune cell infiltration, and hypertrophy of the mucosa muscularis (FIG. 12D). This pathology was largely reversed in mice administered with all doses of rhLF, particularly evident in the highest dose treatment group, 500 mg/kg (DSS+LF500).

To assess molecular readouts of protection, cytokine secretion from small explants of terminal colon were assessed, as described above. A trend towards decreased cytokine secretion (IL-10, TNF, IFNγ) from ex vivo tissue was observed at the highest dose of rhLF gavage (data not shown).

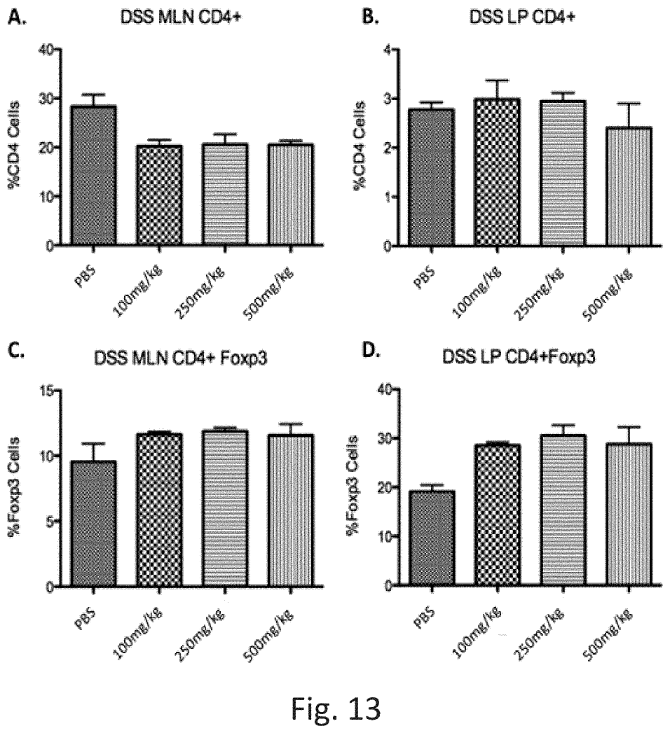

By investigating the burden of all CD4⁺ T cell subtypes, rhLF was demonstrated to reduce cellularity at the draining lymph nodes (MLN), but not at the local site of inflammation (LP) (FIGS. 13A and 13B). This may be indicative of the late stage response of accumulation of CD4⁺ cells at the LP in the DSS model, which may be evident in a more chronic disease, or at a later time point sampling. However, of the populations of CD4⁺ T cells that are present at both tissue sites, an increase in Foxp3⁺ CD4⁺ T cells was observed, indicating an induced population of Tregs in response to rhLF administration (FIGS. 13C and 13D). To demonstrate an effect of rhLF in decreasing proinflammatory T cell production, flow cytometry was used to further detect CD4⁺ T cell populations expressing a variety of cytokines.

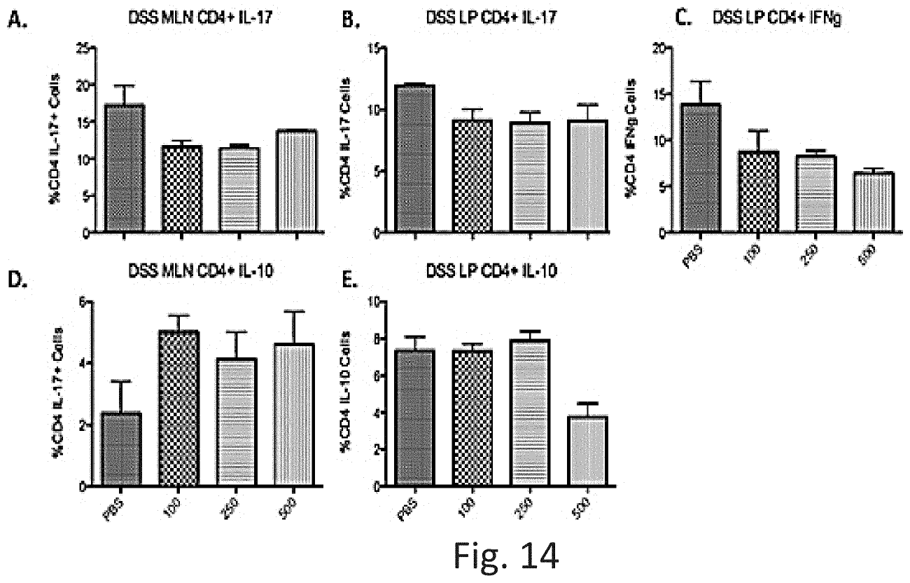

Treatment with rhLF at all doses results in a decrease in number of CD4⁺ T cells that express IL-17 and IFNγ (FIG. 14A-C), as well as a concomitant increase in CD4⁺ cells that produce the anti-inflammatory IL-10, which was evident in the MLN, but not the LP of rhLF-treated mice (FIGS. 14D and 14E). The lack of CD4⁺IL-10⁺ induction in the lamina propria of the rhLF-treated mice may be representative of a resolution of inflammation at this site, and may require investigation of this population of cells at an earlier stage. However, the increase in this population of cells at the MLN suggests a temporospational phenomenon of T cells migrating away from the local site of injury to the draining lymph nodes following resolution of inflammation. Taken together, these data indicate an overall decrease in inflammation and subsequent decrease in inflammatory cytokine output at the source of inflammation. Thus, a protective effect of rhLF in a DSS model of colitis is herein identified, via the observation of skewing of T cell phenotype away from a pro-inflammatory Th1/17 towards a regulatory Treg phenotype.

These data demonstrate for the first time that rhLF acts directly to modulate the immune response in a TNF-driven model of Crohn's like ileitis and DSS induced colitis, with rescue of normal intestinal physiology as demonstrated by enhanced gut barrier function. Furthermore, rhLF reverses the severe chronic-stage pathology seen in both TNF^{ΔARE} and DSS mice, with reversal of tissue damage and a decrease in associated T cell infiltration. Therefore, rhLF decreases inflammation in a DSS model of colitis through skewing the phenotype of CD4⁺ cells away from a proinflammatory Th1/17 phenotype, towards a regulatory Treg phenotype.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. A method of modulating an immune response in a subject, said method comprising administering a composition comprising a plant-expressed recombinant human lactoferrin (rhLF) to a subject in need thereof, wherein said rhLF inhibits pro-inflammatory cytokines and concomitantly stimulates anti-inflammatory cytokines in said subject after said administration.

2. The method of claim 1, wherein said rhLF inhibits the production of TNF, IL-6 and/or IL-1β in said subject after said administration.

3. The method of claim 1, wherein said rhLF stimulates IL-10 and/or IL-4 in said subject after said administration.

4. The method of claim 1, wherein said rhLF is a polypeptide comprising the amino acid sequence of SEQ ID NO: 4, wherein said rhLF composition comprises 0.1% or more plant-derived components.

5. The method of claim 1, wherein said rhLF induces naïve T cells to a Treg phenotype and/or activity in said subject after said administration.

6. The method of claim 5, wherein said rhLF upregulates genes responsible for generation of Treg cells and downregulates genes responsible for generation of IL-17 and Th17 cells, thereby inducing said naïve T cells to said Treg phenotype and/or activity in said subject.

7. The method of claim 5, wherein said rhLF promotes CD4⁺CD25⁻ naïve T cells toward a pro-regulatory phenotype, as measured by intracellular cytokine staining to observe an increase in IFNγ and IL-10⁺ cells in said subject after said administration.

8. The method of claim 1, wherein said rhLF reduces a Th1/Th17 T cell phenotype in said subject after said administration.

9. The method of claim 1, wherein said rhLF modulates a balance between anti-inflammatory Th2 cytokine producing cells and pro-inflammatory Th1 or Th17 cells in said subject after said administration.

10. The method of claim 1, wherein said rhLF upregulates one or more genes involved in Treg generation: Fosl1, Foxp3, Ikzf2, Irf1, Irf4, Tgif, and concomitantly downregulates one or more genes involved in regulating Th17 phenotype: Il-17a, Il17re, Rora, in said subject after said administration.

11. The method of claim 1, wherein modulation of said immune response induces or maintains remission of a neurodegenerative or autoimmune disorder in said subject after said administration.

12. The method of claim 1, wherein modulation of said immune response treats or ameliorates a neurodegenerative or autoimmune disorder in said subject after said administration.

13. The method of claim 12, wherein said neurodegenerative or autoimmune disorder is inflammatory bowel disease (IBD), myotrophic lateral sclerosis (ALS), Alzheimers disease, or rheumatoid arthritis (RA).

14. The method of claim 1, wherein modulation of said immune response suppresses overactivation of an immune response in T-cell (CAR-T) therapy in said subject after said administration.

15. The method of claim 1, wherein said rhLF is administered to a subject at risk of exposure to a virus or other infectious agent.

16. The method of claim 15, wherein said virus or other infectious agent is one associated with subsequent development of an autoimmune disease or disorder, wherein said administration of rhLF ameliorates onset of the disorder in said subject after said administration.

17. The method of claim 1, wherein said rhFL is administered therapeutically to achieve therapeutic benefit in said subject after said administration.

18. The method of claim 1, wherein said rhFL is administered prophylactically to achieve prophylactic benefit in said subject after said administration.

19. The method of claim 1, wherein said rhFL is administered via oral administration, inhalation, enteral administration, feeding, or inoculation by intravenous injection.

20. The method of claim 1, wherein said rhLF is expressed from a monocot plant.
